(19) United States
(12) Patent Application Publication
(10) Pub. No.: US 2024/0042197 A1
(43) Pub. Date: Feb. 8, 2024

(54) IMPLANTABLE ELECTRICAL LEADS AND ASSOCIATED DELIVERY AND CONTROL SYSTEMS

(71) Applicant: ATACOR MEDICAL, INC., San Clemente, CA (US)

(72) Inventors: Rick SANGHERA, San Clemente, CA (US); Matthew ROLLINS, Lafayette, CO (US); Brent CADAVONA, San Diego, CA (US); Rahul VEETEKAT, San Francisco, CA (US); Sterling ERICKSON, St. Paul, MN (US); Alan MARCOVECCHIO, San Clemente, CA (US); Jonathan FREUND, Woburn, MA (US); John BECK, Ramsey, MN (US); William BORN, Ostego, MN (US); Alex GUSTAFSON, Minneapolis, MN (US); Jesse SCHMIDT, St. Paul, MN (US)

(73) Assignee: ATACOR MEDICAL, INC., San Clemente, CA (US)

(21) Appl. No.: 18/365,918

(22) Filed: Aug. 4, 2023

Related U.S. Application Data
(60) Provisional application No. 63/395,283, filed on Aug. 4, 2022.

(51) Int. Cl. A61N 1/05

(52) U.S. Cl. CPC A61N 1/0563; A61N 1/057; A61N 2001/058

(57) ABSTRACT

Systems, methods, devices and computer software for delivering electrical stimulation to biological tissue are described. In some implementations, an electrical lead for implantation in a patient can include a distal portion with electrodes that are configured to generate therapeutic energy for biological tissue of the patient, such as the heart or pericardium. The distal portion can include an electrode extension having a tip electrode, the electrode extension configured to facilitate contact of the tip electrode with biological tissue of the patient when the lead is in a deployed configuration. A distal part of the lead can be configured to include a heel portion, a bend in a proximal part, and/or a bend in a distal part, to facilitate contact of an electrode located on the heel portion with biological tissue of the patient when the lead is in a deployed configuration.

### RELATED APPLICATION(S)

This application claims priority to and the benefit of U.S. Provisional Application No. 63/395,283, filed Aug. 4, 2022, titled “Implantable Electrical Leads and Associated Delivery and Control Systems,” which is hereby incorporated by reference.

### DESCRIPTION OF THE RELATED ART

Electrical leads can be implanted in patients for a variety of medical purposes. In one particular application, leads can be implanted to work in conjunction with a cardiac pacemaker or cardiac defibrillator. Pacemakers and cardiac defibrillators are medical devices that help control abnormal heart rhythms. A pacemaker uses electrical pulses to prompt the heart to beat at a normal rate. The pacemaker may speed up a slow heart rhythm, control a fast heart rhythm, and/or coordinate the chambers of the heart. Defibrillators can be provided in patients who are expected to, or have a history of, severe cardiac problems that may require electrical therapies up to and including the ceasing of ventricular fibrillation, otherwise known as cardiac arrest. Defibrillators may include leads that are physically inserted into the heart, including into the heart tissue (e.g., with screw-in lead tips) for the direct delivery of electrical current to the heart muscle.

The portions of pacemaker or ICD systems generally comprise three main components: a pulse generator, one or more wires called leads, and electrode(s) found on each lead. The pulse generator produces the electrical signals that help regulate the heartbeat. Most pulse generators also have the capability to receive and respond to signals that come from the heart. Leads are generally flexible wires that conduct electrical signals from the pulse generator toward the heart. One end of the lead is attached to the pulse generator and the other end of the lead, containing the electrode(s) is positioned on, in or near the heart.

While many of the exemplary embodiments discussed herein refer to cardiac pacing, it is contemplated that such embodiments and technologies disclosed may also be used in conjunction with defibrillation/ICD applications. Similarly, when exemplary embodiments discussed herein refer to defibrillation/ICD applications, it is contemplated that the embodiments and technologies disclosed may also be used in conjunction with cardiac pacing applications.

### SUMMARY

Systems, methods, devices and computer software for delivering electrical stimulation to biological tissue are described. In some implementations, an electrical lead for implantation in a patient can include a distal portion with electrodes that are configured to generate therapeutic energy for biological tissue of the patient, such as the heart or pericardium. The electrical lead can have a proximal portion coupled to the distal portion and configured to engage a controller configured to cause the electrodes to generate the therapeutic energy, which controller can be run by computer software.

Disclosed in detail herein are numerous implementations of lead designs, electrode designs, delivery systems, delivery system accessories to facilitate implementation, systems for securing leads to a patient, electrical stimulation control systems, software and sensors to work with control systems, etc.

In some implementations, there can be multiple electrodes and the distal portion of the lead can include an electrode extension having a tip electrode, the electrode extension configured to facilitate contact of the tip electrode with biological tissue of the patient when the lead is in a deployed configuration.

The distal portion of the lead includes a cavity in a proximal part and/or distal part of the distal portion that is shaped to receive the electrode extension when the lead is in a loaded configuration. The electrode extension can be coupled to a distal part of the distal portion and, in the deployed configuration, extending at an angle away from the distal part. The electrode extension can be coupled to a proximal part of the distal portion and, in the deployed configuration, extending at an angle away from the distal part. The electrode extension can further include an elbow. The electrode extension can be coupled to a proximal part of the distal portion and, in the deployed configuration having a horizontal extension and a vertical extension. The electrode extension can be coupled to a proximal part of the distal portion and, in the deployed configuration, having a C-shape and comprising a vertical extension. The electrode extension can be coupled to a proximal part of the distal portion and, in the deployed configuration, the electrode extension ending flush with a distal part of the distal portion with only the tip electrode protruding beyond the distal part. The electrode extension can be coupled to a distal part of the distal portion and, in the deployed configuration, extending substantially coplanar to the distal part. The electrode extension can be coupled to and aligned with a distal part of the distal portion. The electrode extension can be wider than a width of the tip electrode.

In some implementations, a distal part of the lead can be configured to include a heel portion to facilitate contact of an electrode located on the heel portion with biological tissue of the patient when the lead is in a deployed configuration.

The heel portion may be formed by a bend in the distal part of the lead that facilitates contact of the electrode located on the heel portion with the biological tissue of the patient when the lead is in the deployed configuration.

A proximal part can include a bend to place a vertical portion of the proximal part closer to a distal tip of the lead when the lead is in a deployed configuration to facilitate contact of an electrode with biological tissue of the patient when the lead is in the deployed configuration. The bend can place the vertical portion approximately over an electrode on the distal part. The bend can place the vertical portion closer to the distal tip than an electrode on the distal part. The proximal part can include an S-shape.

In some implementations, the bend can be configured to increase the flexibility of the proximal part of the lead to facilitate maintaining contact with the biological tissue when the lead is in the deployed configuration.

In some implementations, the proximal part can include one or more grooves or holes for suturing the vertical portion to the patient.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating exemplary placements of elements of a cardiac pacing system, in accordance with certain aspects of the present disclosure;

FIG. 2A is an illustration of an exemplary lead delivery system facilitating delivery of a cardiac pacing lead in the region of a cardiac notch, in accordance with certain aspects of the present disclosure;

FIG. 2B illustrates a distal end of an exemplary lead delivery system having dropped into an intercostal space in the region of the cardiac notch, in accordance with certain aspects of the present disclosure;

FIG. 2C illustrates an electrical lead exiting the exemplary delivery system with two electrodes positioned on a side of the lead facing the heart, in accordance with certain aspects of the present disclosure;

FIG. 3 illustrates an exemplary delivery system, in accordance with certain aspects of the disclosure;

FIG. 4 illustrates an example of first and second insertion tips of the delivery system with blunt edges, in accordance with certain aspects of the disclosure;

FIG. 5 illustrates an exemplary channel at least partially complimentary to a shape of the component and configured to guide the component into the patient, in accordance with certain aspects of the disclosure;

FIG. 6 illustrates a first insertion tip being longer than a second insertion tip, in accordance with certain aspects of the disclosure;

FIG. 7 illustrates an example of a ramped portion of an insertion tip, in accordance with certain aspects of the disclosure;

FIG. 8 illustrates an example of insertion tips with open side walls, in accordance with certain aspects of the disclosure;

FIG. 9A illustrates one possible example of a delivery system having a unitary insertion tip, in accordance with certain aspects of the disclosure;

FIG. 9B illustrates one possible example of a unitary insertion tip, in accordance with certain aspects of the disclosure;

FIG. 9C illustrates an alternative insertion tip design having a wedge shape, in accordance with certain aspects of the disclosure;

FIG. 9D illustrates certain features applicable to a unitary insertion tip design, in accordance with certain aspects of the disclosure;

FIG. 10 illustrates an exemplary lock for a delivery system, in a locked position, in accordance with certain aspects of the disclosure;

FIG. 11 illustrates the lock in an unlocked position, in accordance with certain aspects of the disclosure;

FIG. 12A illustrates an example rack and pinion system that may be included in a component advancer of the delivery system, in accordance with certain aspects of the disclosure;

FIG. 12B illustrates an example clamp system that may be included in a component advancer of the delivery system, in accordance with certain aspects of the disclosure;

FIG. 13 illustrates a view of an exemplary implementation of a component advancer including a pusher tube coupled with the handle of a delivery system, in accordance with certain aspects of the disclosure;

FIG. 14 illustrates another view of the exemplary implementation of the component advancer including the pusher tube coupled with the handle of the delivery system, in accordance with certain aspects of the disclosure;

FIG. 15 illustrates the exemplary insertion tips in an open position, in accordance with certain aspects of the disclosure;

FIG. 16 illustrates an example implementation of an electrical lead, in accordance with certain aspects of the disclosure;

FIG. 17 illustrates another example implementation of an electrical lead, in accordance with certain aspects of the disclosure;

FIG. 18 illustrates a distal portion of an exemplary electrical lead bent in a predetermined direction, in accordance with certain aspects of the disclosure;

FIG. 19 illustrates the distal portion bending in the predetermined direction when the lead exits the delivery system, in accordance with certain aspects of the disclosure;

FIG. 20A illustrates an exemplary implementation of the distal portion of a lead, in accordance with certain aspects of the disclosure;

FIG. 20B illustrates another exemplary implementation of the distal portion of a lead, in accordance with certain aspects of the disclosure, in accordance with certain aspects of the disclosure;

FIG. 21A is a simplified diagram illustrating an exemplary junction box in accordance with certain aspects of the present disclosure;

FIG. 21B is a flow chart illustrating an exemplary process for performing defibrillation in accordance with certain aspects of the disclosure;

FIG. 22 illustrates an example of an electrode, in accordance with certain aspects of the disclosure;

FIG. 23 illustrates a cross section of the example electrode, in accordance with certain aspects of the disclosure;

FIG. 24 is a diagram illustrating a simplified perspective view of an exemplary directional lead with panel electrodes in accordance with certain aspects of the present disclosure;

FIG. 25A is a diagram illustrating a simplified perspective view of an exemplary directional lead with elliptical panel electrodes in accordance with certain aspects of the present disclosure;

FIG. 25B is a diagram illustrating a simplified perspective view of an exemplary directional lead with elliptical coil electrodes in accordance with certain aspects of the present disclosure;

FIG. 26 is a diagram illustrating a simplified perspective view of an exemplary directional lead with embedded directional electrodes in accordance with certain aspects of the present disclosure;

FIG. 27 is a diagram illustrating a simplified perspective view of an exemplary directional lead with masked circumferential defibrillation coil electrodes in accordance with certain aspects of the present disclosure;

FIG. 28A illustrates an exemplary lead including electrodes that are angled and offset, in accordance with certain aspects of the present disclosure;

FIG. 28B illustrates an exemplary lead including an electrode at least partially on the side of the lead, in accordance with certain aspects of the present disclosure;

FIG. 28C illustrates an exemplary lead including radiopaque indicators, in accordance with certain aspects of the present disclosure;

FIG. 29A illustrates an exemplary lead including a balloon for applying a downward force to a distal portion of the lead, in accordance with certain aspects of the present disclosure;

FIG. 29B illustrates an exemplary lead including a wedge for applying a downward force to a distal portion of the lead, in accordance with certain aspects of the present disclosure;

FIG. 29C illustrates an exemplary lead having an elastically deformable portion configured to have one point of contact for pushing against a chest wall, in accordance with certain aspects of the present disclosure;

FIG. 29D illustrates an exemplary lead configured to have two points of contact for pushing against a chest wall, in accordance with certain aspects of the present disclosure;

FIG. 29E illustrates an exemplary lead including a suction cup for pulling the lead against biological tissue, in accordance with certain aspects of the present disclosure;

FIG. 29F illustrates an exemplary lead that includes tines for pulling the lead against biological tissue, in accordance with certain aspects of the present disclosure;

FIG. 29G illustrates an exemplary lead having a coiled shape, in accordance with certain aspects of the present disclosure;

FIG. 29H illustrates an exemplary lead having a spiral shape, in accordance with certain aspects of the present disclosure;

FIG. 29I illustrates an exemplary lead having a wavy shape, in accordance with certain aspects of the present disclosure;

FIG. 29J illustrates an exemplary lead having an electrode extension, in accordance with certain aspects of the present disclosure;

FIGS. 29K-S illustrate exemplary leads having electrode extensions, in accordance with certain aspects of the present disclosure;

FIG. 29T illustrates an exemplary lead having two sub-portions, in accordance with certain aspects of the present disclosure;

FIG. 29U illustrates an exemplary lead having three sub-portions, in accordance with certain aspects of the present disclosure;

FIG. 29V illustrates an exemplary lead having electrodes on separate sub-portions, in accordance with certain aspects of the present disclosure;

FIG. 29W illustrates an exemplary lead having an electrode on one sub-portion and laterally-extending portions on that same sub-portion, in accordance with certain aspects of the present disclosure;

FIG. 29X illustrates an exemplary lead having two sub-portions, in accordance with certain aspects of the present disclosure;

FIG. 29Y illustrates an exemplary lead having a heel portion, in accordance with certain aspects of the present disclosure;

FIG. 29Y-1 illustrates an exemplary lead having a heel portion and a bend in a vertical portion of the proximal part of the lead, in accordance with certain aspects of the present disclosure;

FIG. 29Y-2 illustrates an exemplary lead having a heel portion and an S-shape in the proximal part of the lead, in accordance with certain aspects of the present disclosure;

FIGS. 30A and 30B illustrate an exemplary splitting lead exiting a delivery system, in accordance with certain aspects of the present disclosure;

FIGS. 31A and 31B illustrate exemplary implantation locations/orientations for exemplary splitting leads, in accordance with certain aspects of the present disclosure;

FIG. 32 illustrates an exemplary splitting lead exiting an exemplary delivery system, in accordance with certain aspects of the present disclosure;

FIG. 33A illustrates an exemplary splitting lead with wrapped electrodes, in accordance with certain aspects of the present disclosure;

FIG. 33B illustrates an exemplary splitting lead that includes proximally placed cathodes, in accordance with certain aspects of the present disclosure;

FIG. 33C illustrates an exemplary splitting lead that includes distally placed cathodes, in accordance with certain aspects of the present disclosure;

FIG. 33D illustrates an exemplary splitting lead that includes electrodes between segments of a defibrillation electrode, in accordance with certain aspects of the present disclosure;

FIG. 34A illustrates an exemplary splitting lead with an electrode extension, in accordance with certain aspects of the present disclosure;

FIG. 34B illustrates an exemplary splitting lead with a flexible electrode extension, in accordance with certain aspects of the present disclosure;

FIG. 35A illustrates an exemplary embodiment of a splitting lead that includes a protective collar for an electrode on an electrode extension, in accordance with certain aspects of the present disclosure;

FIG. 35B illustrates an exemplary embodiment with an electrode on a bridge between two sub-portions of a splitting lead, in accordance with certain aspects of the present disclosure;

FIG. 36 illustrates an exemplary splitting lead with an embedded circular helical coil electrode, in accordance with certain aspects of the present disclosure;

FIG. 37 illustrates an exemplary splitting lead with an embedded elliptical helical coil electrode, in accordance with certain aspects of the present disclosure;

FIG. 38 illustrates an exemplary splitting lead with multiple embedded electrodes, in accordance with certain aspects of the present disclosure;

FIG. 39 illustrates an exemplary splitting lead with multiple side-by-side embedded electrodes, in accordance with certain aspects of the present disclosure;

FIG. 40A illustrates an exemplary splitting lead with offset embedded electrodes, in accordance with certain aspects of the present disclosure;

FIG. 40B illustrates an exemplary splitting lead with offset embedded electrodes that fit into opposing concavities, in accordance with certain aspects of the present disclosure;

FIG. 41A illustrates an exemplary delivery system deploying a component, in accordance with certain aspects of the present disclosure;

FIG. 41B illustrates the delivery system of FIG. 41A at a later stage of deployment, in accordance with certain aspects of the present disclosure;

FIG. 41C illustrates the delivery system of FIG. 41A at a yet later stage of deployment, in accordance with certain aspects of the present disclosure;

FIG. 41D illustrates an exemplary gap-filling component of a splitting lead for use with a delivery system such as depicted in FIGS. 41A-C, in accordance with certain aspects of the present disclosure;

FIG. 41E illustrates an exemplary component having transition portions to aid in withdrawal of the component from a patient, in accordance with certain aspects of the present disclosure;

FIG. 42 illustrates exemplary components of a delivery system configured to load (or reload) a component (e.g., an electrical lead) into the delivery system, in accordance with certain aspects of the disclosure;

FIG. 43 illustrates an example of an alignment block coupled to a proximal portion of an electrical lead, in accordance with certain aspects of the disclosure;

FIG. 44A illustrates an exemplary insertion dilator and insertion sheath, in accordance with certain aspects of the disclosure;

FIGS. 44B and 44C illustrate an exemplary use and structure of a puncture tip for an insertion dilator, in accordance with certain aspects of the disclosure;

FIG. 44D illustrates an exemplary recessed button for the insertion dilator, in accordance with certain aspects of the disclosure;

FIG. 44E illustrates a delivery system with an exemplary dilator cap, in accordance with certain aspects of the disclosure;

FIG. 45 illustrates removal of the insertion dilator from the insertion sheath, in accordance with certain aspects of the disclosure;

FIG. 46 illustrates exemplary features of a lead delivery system that facilitate loading a splitting lead into the insertion tip of the system, in accordance with certain aspects of the disclosure;

FIG. 47A illustrates utilization of a delivery system having an insertion tip that is inserted into an insertion sheath, in accordance with certain aspects of the disclosure;

FIG. 47B illustrates exemplary embodiments of insertion sheaths, in accordance with certain aspects of the disclosure;

FIG. 48 illustrates deployment of a splitting lead, in accordance with certain aspects of the disclosure;

FIG. 49 illustrates the insertion sheath creating a reduced window size that improves deployment of the splitting lead, in accordance with certain aspects of the disclosure;

FIG. 50 illustrates removal of the delivery system and insertion tip, in accordance with certain aspects of the disclosure;

FIGS. 51-53 illustrate removal of an insertion sheath embodiment having separating portions, in accordance with certain aspects of the disclosure;

FIG. 54 illustrates a lead with exemplary suture holes, in accordance with certain aspects of the present disclosure;

FIG. 55 illustrates an exemplary lead anchor for securing a lead, in accordance with certain aspects of the present disclosure;

FIG. 56 illustrates an exemplary lead anchor insertion tool for pushing a lead anchor onto a lead, in accordance with certain aspects of the present disclosure;

FIG. 57A illustrates an exemplary lead with indentations for securing the lead to tissue, in accordance with certain aspects of the present disclosure; and

FIG. 57B illustrates an exemplary lead and anchor cap, in accordance with certain aspects of the present disclosure.

### DETAILED DESCRIPTION

Implantable medical devices such as cardiac pacemakers or implantable cardioverter defibrillators (ICDs) may provide therapeutic electrical stimulation to the heart of a patient. The electrical stimulation may be delivered in the form of electrical pulses or shocks for pacing, cardioversion or defibrillation. This electrical stimulation is typically delivered via electrodes on one or more implantable leads that are positioned in, on or near the heart. The concepts described herein can be applied to leads that include pacing and/or defibrillation electrodes unless otherwise specifically stated.

In one particular implementation discussed herein, a lead may be inserted in the region of the cardiac notch of a patient so that the distal end of the lead is positioned within the mediastinum, adjacent to the heart. For example, the distal end of the lead may be positioned in the anterior mediastinum, beneath the patient's sternum. The distal end of the lead can also be positioned so to be aligned with an intercostal space in the region of the cardiac notch. Other similar placements in the region of the cardiac notch, adjacent the heart, are also contemplated for this particular application of cardiac pacing.

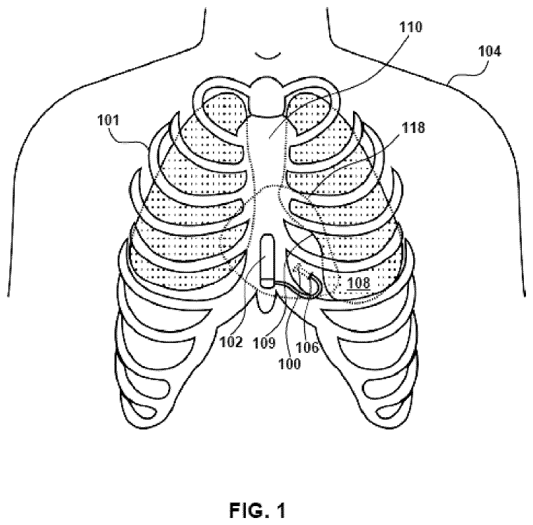

In one exemplary procedure, as shown in FIG. 1, a cardiac pacing lead 100 may be inserted within the ribcage 101 of a patient 104 through an intercostal space 108 in the region of the cardiac notch. Lead 100 may be inserted through an incision 106, for example. The incision 106 may be made in proximity to the sternal margin to increase the effectiveness in finding the appropriate intercostal space 108 and avoiding certain anatomical features, for example the lung 109. The incision may be made lateral to the sternal margin, adjacent the sternal margin or any other direction that facilitates access to an appropriate intercostal space 108. A distal end of lead 100 can be positioned to terminate within the mediastinum of the thoracic cavity of the patient, proximate the heart 118. Lead 100 may then be connected to a pulse generator or controller 102, which may be placed above the patient's sternum 110. In alternative procedures, for temporary pacing, a separate controller may be used that is not implanted in the patient.

In some implementations, the pericardium is not invaded by the lead during or after implantation. In other implementations, incidental contact with the pericardium may occur, but heart 118 (contained within the pericardium) may remain untouched. In still further procedures, epicardial leads, or leads that reside within the pericardium, which do invade the pericardium, may be inserted.

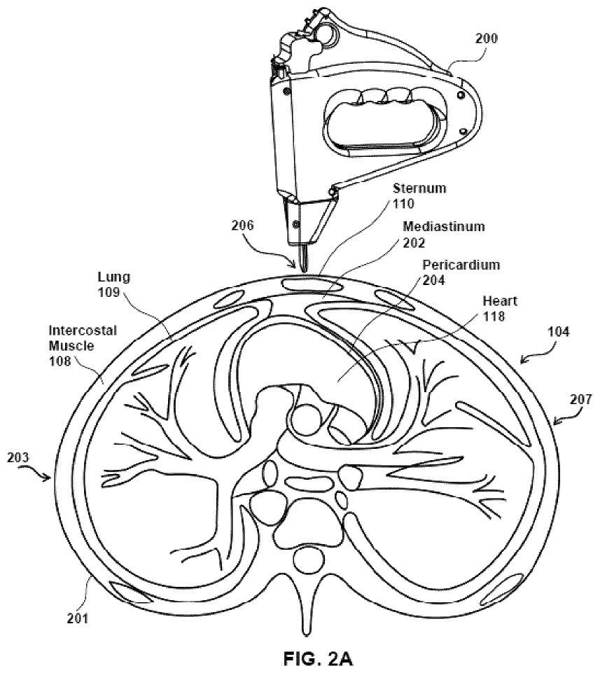

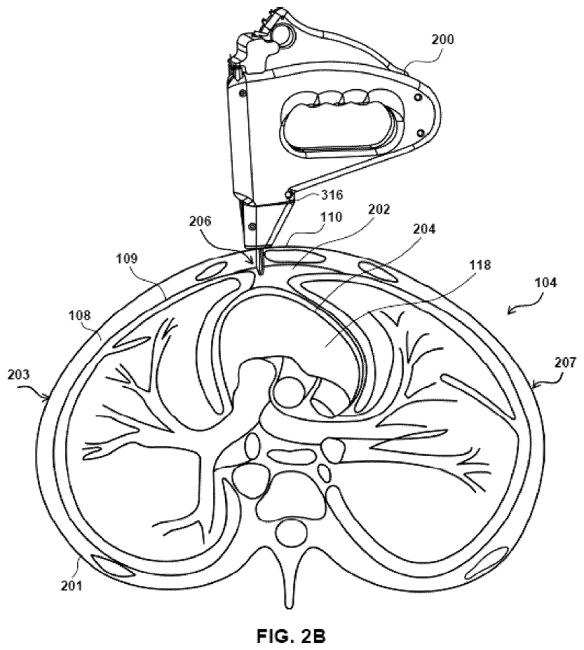

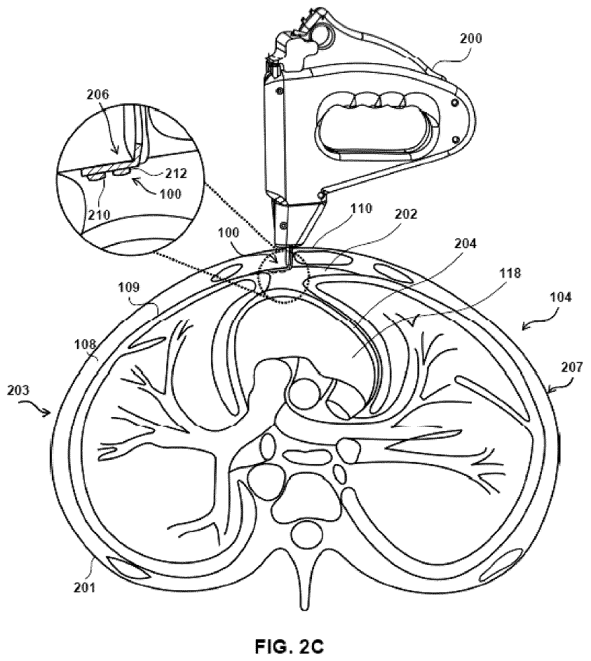

FIG. 2A is an illustration of an exemplary lead delivery system 200 facilitating delivery of a lead in the region of a cardiac notch. FIG. 2A illustrates delivery system 200 and a cross section 201 (including left chest 203 and right chest 207) of a patient 104. FIG. 2A illustrates sternum 110, lung 109, intercostal muscle 108, heart 118, mediastinum 202, pericardium 204, and other anatomical features. As shown in FIG. 2A, lead delivery system 200 may be configured to allow for a distal end 206 of delivery system 200 to be pressed against the sternum 110 of patient 104.

In one implementation, a physician identifies an insertion point above or adjacent to a patient's sternum 110 and makes an incision. The distal end 206 of delivery system 200 can then be inserted through the incision, until making contact with sternum 110. The physician can then slide distal end 206 of delivery system 200 across sternum 110 toward the sternal margin until it drops through the intercostal muscle 108 in the region of the cardiac notch under pressure applied to the delivery system 200 by the physician. FIG. 2B illustrates the distal end 206 having dropped through the intercostal muscle in the region of the cardiac notch toward the pericardium.

In certain implementations, delivery system 200 may include an orientation or level guide 316 to aid the physician with obtaining the proper orientation and/or angle of delivery system 200 to the patient. Tilting delivery system 200 to the improper angle may negatively affect the deployment angle of lead 100 into the patient. For example, a horizontal level guide 316 on delivery system 200 helps to ensure that the physician keeps delivery system 200 level with the patient's sternum thereby ensuring lead 100 is delivered at the desired angle.

Following this placement of delivery system 200, the system may be actuated to insert an electrical lead 100 into the patient. FIG. 2C illustrates an exemplary electrical lead 100 exiting delivery system 200 with two electrodes 210, 212 positioned on one side of lead 100, within the mediastinum 202 and facing heart 118. FIG. 2C illustrates the lead 100 advancing in a direction away from sternum 110. This example is not intended to be limiting. For example, the lead 100 may also be advanced in a direction parallel to the sternum 110. In some implementations, delivery system 200 may be configured such that lead 100 advances in the opposite direction, under sternum 110, advances away from sternum 110 at an angle that corresponds to an angle of one or more ribs of patient 104, and/or advances in other orientations. Similarly, an exemplary device as shown in FIG. 2 may be flipped around so that the handle would be on the left side of FIG. 2, or held in other positions by the physician, prior to system actuation and insertion of lead 100.

Distal end 206 of delivery system 200 may be configured to move or puncture tissue during insertion, for example, with a relatively blunt tip (e.g., as described herein), to facilitate entry into the mediastinum without requiring a surgical incision to penetrate through intercostal muscles and other tissues. A blunt access tip, while providing the ability to push through tissue, can be configured to limit the potential for damage to the pericardium or other critical tissues or vessels that the tip may contact.

In an exemplary implementation, the original incision made by the physician above or adjacent to the sternum may also be used to insert a controller, pulse generator or additional electrode to which the implanted lead may be connected.

The delivery system and lead technologies described herein may be especially well suited for the cardiac pacing lead delivery example described above. While this particular application has been described in detail, and may be utilized throughout the descriptions below, it is contemplated that the delivery system(s) 200 and lead(s) 100 herein may be utilized in other procedures as well, such as the insertion of a defibrillation lead.

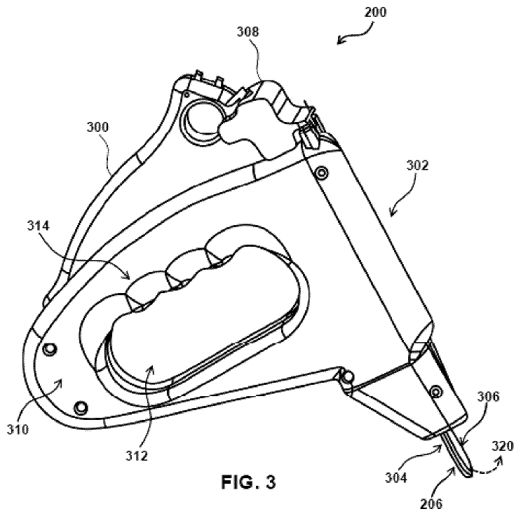

FIG. 3 illustrates an exemplary delivery system 200. Delivery system 200 can include a handle 300, a component advancer 302, a first insertion tip 304, a second insertion tip 306, a lock 308, and/or other components. Handle 300 may be configured to be actuated by an operator. In some implementations, handle 300 may be coupled to a body 310 and/or other components of delivery system 200. Body 310 may include an orifice 312, finger depressions 314, a knurled surface, a lever arm, and/or other components configured to facilitate gripping of handle 300 by an operator. In some implementations, handle 300 and the body of the delivery system 200 may be coated with a material or their surfaces covered with a texture to prevent slippage of the physician's grasp when using delivery system 200.

Component advancer 302 may be coupled to handle 300 and configured to advance a component such as an electrical lead (as one example) into the patient by applying a force to the portion of the component in response to actuation of handle 300 by the operator.

First insertion tip 304 and second insertion tip 306 may be configured to close around a distal tip and/or segment of the component when the component is placed within component advancer 302. In some implementations, closing around a distal segment of the component may include blocking a path between the component and the environment outside delivery system 200. Closing around the distal segment of the component may also prevent the component from being unintentionally deployed and contacting biological tissue while delivery system 200 is being manipulated by the operator.

First insertion tip 304 and second insertion tip 306 may also be configured to fully enclose the distal segment of the component when the component is placed within component advancer 302. Fully enclosing the distal segment of the component may include covering, surrounding, enveloping, and/or otherwise preventing contact between the distal segment of the component and an environment around first insertion tip 304 and second insertion tip 306.

In still other implementations, first insertion tip 304 and second insertion tip 306 may be configured to only partially enclose the distal segment of the component when the component is placed within component advancer 302. For example, first insertion tip 304 and/or second insertion tip 306 may cover, surround, envelop, and/or otherwise prevent contact between one or more portions (e.g., surfaces, ends, edges, etc.) of the distal segment of the component and the environment around tips 304 and 306, but the tips 304 and 306 may also still block the path between the component and the environment outside the delivery system 200 during insertion.

In some implementations, first insertion tip 304 and second insertion tip 306 may be configured such that the component is held within component advancer 302 rather than within first insertion tip 304 and second insertion tip 306, prior to the component being advanced into the patient.

First insertion tip 304 and second insertion tip 306 may be further configured to push through biological tissue when in a closed position and to open (see, e.g., 320 in FIG. 3) to enable the component to exit from the component advancer 302 into the patient. In some implementations, opening may comprise second insertion tip 306 moving away from first insertion tip 304, and/or other opening operations. In some implementations, first and second insertion tips 304, 306 may be configured to open responsive to actuation of handle 300.

In some implementations, first insertion tip 304 and/or second insertion tip 306 may be configured to close (or re-close) after the component exits from the component advancer 302, to facilitate withdrawal of delivery system 200 from the patient. Thus, first insertion tip 304 and second insertion tip 306 may be configured to move, after the component exits from component advancer 302 into the patient, to a withdrawal position to facilitate withdrawal of first insertion tip 304 and second insertion tip 306 from the biological tissue. In some implementations, the withdrawal position may be similar to and/or the same as an original closed position. In some implementations, the withdrawal position may be a different position. In some implementations, the withdrawal position may be wider than the closed position, but narrower than an open position. For example, first insertion tip 304 and/or second insertion tip 306 may move to the open position to release the component, but then move to a different position with a narrower profile (e.g., the withdrawal position) so that when the tips 304, 306 are removed they are not met with resistance pulling through a narrow rib space, and/or other biological tissue.

In some implementations, first and second insertion tips 304, 306 may have blunt edges. Blunt edges may include rounded and/or otherwise dull edges, corners, surfaces, and/or other components of first and second insertion tips 304, 306. The blunt edges may be configured to prevent insertion tips 304 and 306 from rupturing any veins or arteries, the pericardial sac, the pleura of the lungs, and/or causing any other unintentional damage to biological tissue. The blunt edges may prevent, for example, rupturing veins and/or arteries by pushing these vascular items to the side during insertion. The blunt edges may also prevent, for example, the rupturing of the pericardium or pleura because they are not sharp.

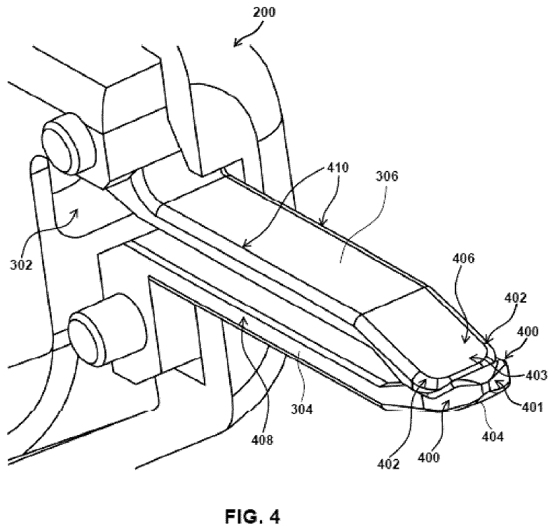

FIG. 4 illustrates first and second insertion tips 304, 306 with exemplary implementations of such blunt edges. As shown in FIG. 4, first and second insertion tips 304, 306 may have rounded corners 400, 402 and/or end surfaces 401, 403 at their respective ends 404, 406. First and second insertion tips 304, 306 may have rounded edges 408, 410 that run along a longitudinal axis of tips 304, 306. However, this description is not intended to be limiting. In some implementations, first and second insertion tips 304, 306 may also have sharp edges, ends, and/or other features.

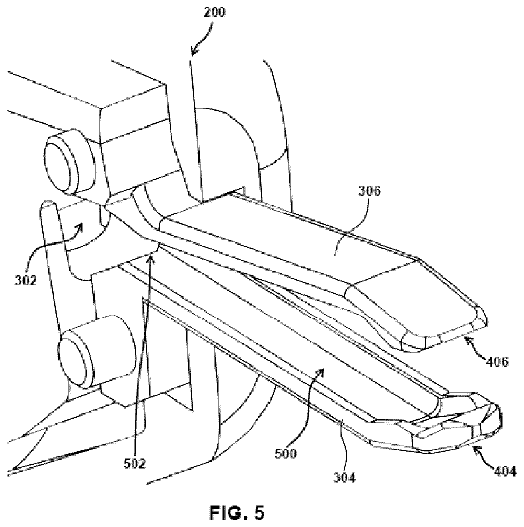

In some implementations, first and second insertion tips 304, 306 may each include a channel at least partially complimentary to a shape of the component and configured to guide the component into the patient. FIG. 5 illustrates an example of such a channel. As shown in FIG. 5, first insertion tip 304 may include a channel 500 at least partially complimentary to a shape of the component and configured to guide the component into the patient. Second insertion tip 306 may also include a channel similar to and/or the same as channel 500 (although the channel in insertion tip 306 is not visible in FIG. 5). Channel 500 may extend along a longitudinal axis of insertion tip 304 from an end 502 of insertion tip 304 configured to couple with component advancer 302 toward end 404.

In some implementations, channel 500 may be formed by a hollow area of insertion tip 304 that forms a trench, for example. The hollow area and/or trench may have one or more shapes and/or dimensions that are at least partially complimentary to a shape and/or dimension(s) of the component, and are configured to guide the component into the patient. In some implementations, the hollow area and/or trench may be configured such that the component may only slide within channel 500 inside the insertion tips 304, 306, and therefore prevent the component from advancing out one of the sides of the insertion tips 304, 306 when pushed by component advancer 302.

In some implementations, channel 500 may include a second channel and/or groove configured to engage alignment features included on a component. The second channel or groove may be located within channel 500, but be deeper and/or narrower than channel 500. The component may then include a rib and/or other alignment features configured to engage such a groove. The rib may be on an opposite side of the component relative to electrodes, for example. These features may enhance the guidance of a component through channel 500, facilitate alignment of a component in channel 500 (e.g., such that the electrodes are oriented in a specific direction in tips 304, 306, preventing the component from exiting tips 304, 306 to one side or the other (as opposed to exiting out ends 404, 406), and/or have other functionalities.

In some implementations, the second channel and/or groove may be sized to be just large enough to fit an alignment feature of the component within the second channel and/or groove. This may prevent an operator from pulling a component too far up into delivery system 200 (FIG. 3) when loading delivery system 200 with a component (e.g., as described below).

The channels and/or grooves may also provide a clinical benefit. For example, the channel and/or groove may allow for narrower insertion tips 304 and 306 that need not be configured to surround or envelop all sides of the component (e.g., they may not need sidewalls to keep the component in position during implantation). If surrounding or enveloping all sides of a component is necessary, the insertion tips would need to be larger, and would meet with greater resistance when separating tissue planes within intercostal spaces, for example. However, in other implementations (e.g., as described herein), insertion tips 304, 306 may completely surround and/or envelop the component.

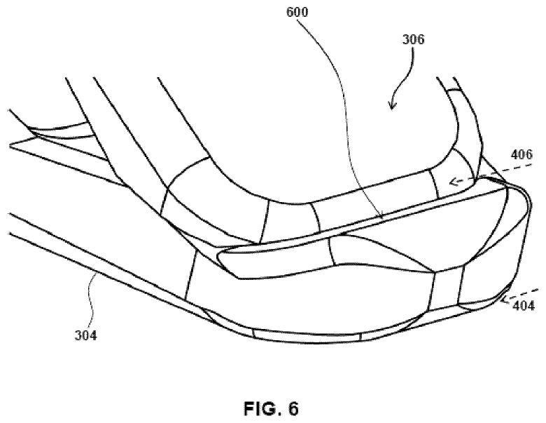

In some implementations, as shown in FIG. 6, a first insertion tip 304 may be longer than a second insertion tip 306 and the end 404 of first insertion tip 304 will extend beyond the end 406 of insertion tip 306. Such a configuration may assist with spreading of tissue planes and help to avoid pinching tissue, veins, arteries or the like while delivery system 200 is being manipulated through biological tissue.

In some implementations, both the first and second insertion tips 304, 306 may be moveable. In other implementations, the first insertion tip 304 may be fixed, and second insertion tip 306 may be moveable.

In one particular implementation, a fixed insertion tip 304 may be longer than a movable insertion tip 306. This configuration may allow more pressure to be exerted on the outermost edge (e.g., end 404 of tip 304) of delivery system 200 without (or with reduced) concern that tips 304 and 306 will open when pushing through biological tissue. Additionally, the distal ends 404 and 406 may form an underbite 600 that allows distal end 406 of movable insertion tip 306 (in this example) to seat behind fixed insertion tip 304, and thus prevent tip 406 from experiencing forces that may inadvertently open movable insertion tip 306 during advancement. However, this description is not intended to be limiting. In some implementations, a movable insertion tip 306 may be longer than a fixed insertion tip 304.

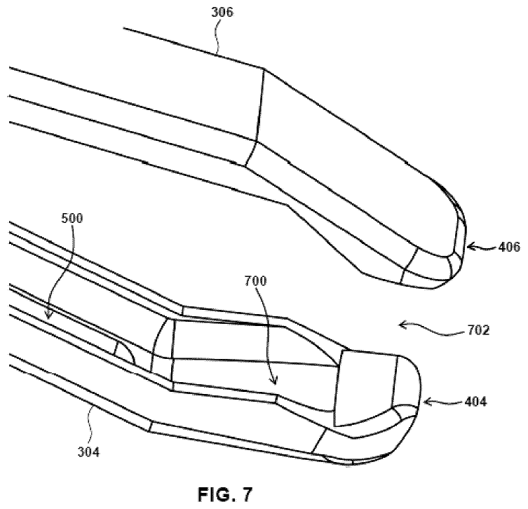

In some implementations, a fixed (e.g., and/or longer) insertion tip 304 may include a ramped portion configured to facilitate advancement of the component into the patient in a particular direction. FIG. 7 illustrates an example of a ramped portion 700 of insertion tip 304. Ramped portion 700 may be located on an interior surface 702 of insertion tip 304, between channel 500 and distal end 404 of insertion tip 304. Ramped portion 700 may be configured to facilitate advancement of the component into the patient in a particular direction. The particular direction may be a lateral direction relative to a position of insertion tip 304, for example. The lateral deployment of a component (e.g., an electrical lead) when it exits insertion tip 304 and moves into the anterior mediastinum of the patient may facilitate deployment without contacting the heart (e.g., as described relative to FIGS. 2A-2C above). Ramped portion 700 may also encourage the component to follow a preformed bias (described below) and help prevent the lead from deploying in an unintentional direction.

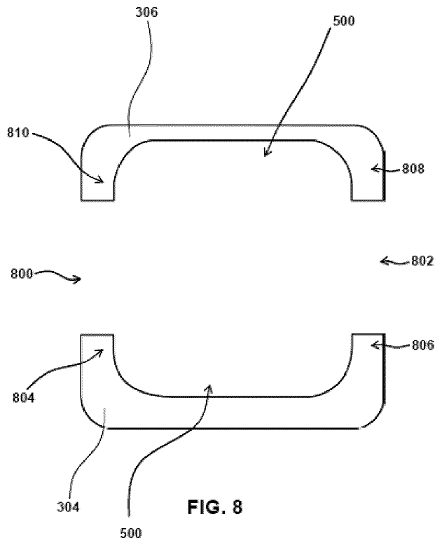

In some implementations, insertion tips 304, 306 may have open side walls. FIG. 8 illustrates an example of insertion tips 304, 306 with open side walls 800, 802. FIG. 8 illustrates a cross sectional view of insertion tips 304, 306, looking at insertion tips 304, 306 from distal ends 404, 406 (as shown in FIG. 7). Open side walls 800, 802 may be formed by spaces between insertion tip 304 and insertion tip 306. In the example of FIG. 8, insertion tips 304 and 306 are substantially “U” shaped, with the ends 804, 806, 808, 810 extending toward each other, but not touching, such that open side walls 800 and 802 may be formed. Open side walls 800, 802 may facilitate the use of a larger component (e.g., a component that does not fit within channel(s) 500), without having to increase a size (e.g., a width, etc.) of insertion tips 304, 306. This may avoid effects larger insertion tips may have on biological tissue. For example, larger insertion tips are more invasive than smaller insertion tips. As such, larger insertion tips may meet with greater resistance when separating tissue planes within intercostal spaces during deployment and may cause increased trauma than insertion tips having a reduced cross sectional size.

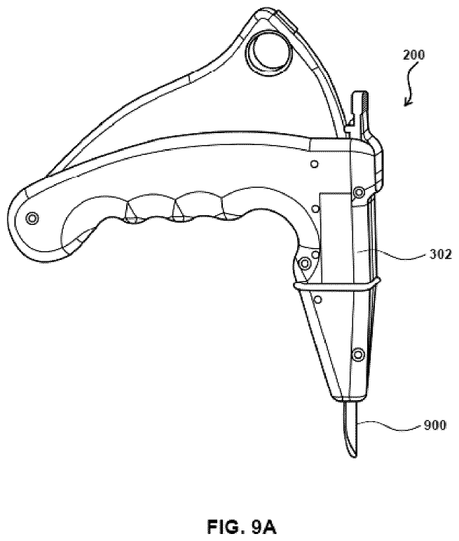

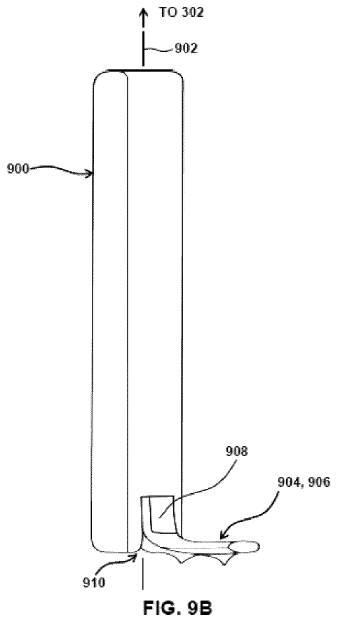

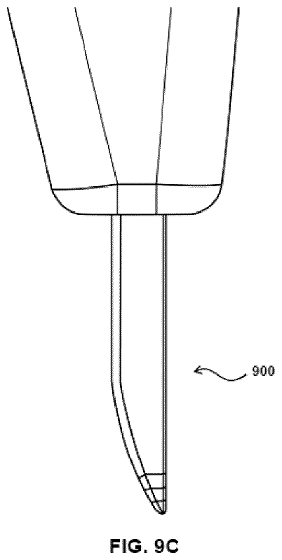

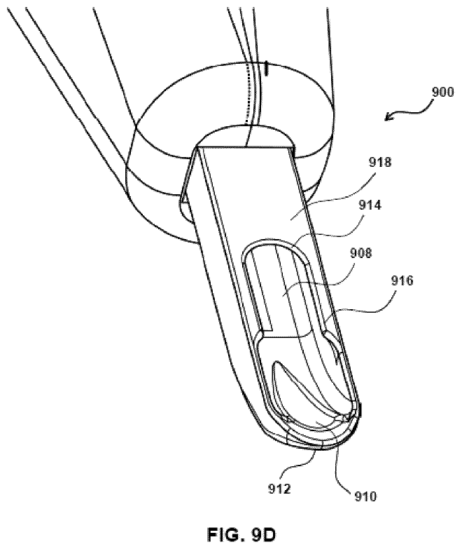

In some implementations, delivery system 200 (FIG. 3) may include a handle 300 (FIG. 3), a component advancer 302 (FIG. 3), and a unitary insertion tip (e.g., instead of first and second insertion tips 304 and 306). FIG. 9A illustrates one possible example of a delivery system 200 having a unitary insertion tip 900. Insertion tip 900 may be coupled to a component advancer 302 similar to and/or in the same manner that insertion tips 304 and 306 (FIG. 7) may be coupled to component advancer 302.

Unitary insertion tip 900 may have a circular, rectangular, wedge, square, and/or other cross sectional shape(s). In some implementations, insertion tip 900 may form a (circular or rectangular, etc.) tube extending along a longitudinal axis 902 (FIG. 9B) of insertion tip 900. Referring to FIG. 9B, in some implementations, insertion tip 900 may be configured to hold the component (labeled as 904) when the component is placed within component advancer 302. In some implementations, insertion tip 900 may be configured to hold a distal end (labeled as 906) and/or tip of component 904 when component 904 is placed within component advancer 302.

Insertion tip 900 may be configured to push through biological tissue and may include a distal orifice 908 configured to enable component 904 to exit from component advancer 302 into the patient.

FIG. 9C illustrates an alternative insertion tip 900 design having a wedge shape. A wedge-shaped insertion tip 900 reduces and/or eliminates the exposure of distal orifice 908 to the surrounding tissue during insertion. This design prevents tissue coring since only the leading edge of insertion tip 900 is exposed and thereby separates tissues rather than coring or cutting tissue during insertion. Accordingly, the present disclosure contemplates an insertion tip that may be configured to reduce the exposure of the distal orifice during insertion.

Referring to FIG. 9D, distal tip 912 may be rounded into an arc so the deployment force exerted by the physician during insertion concentrates in a smaller area (the distalmost portion of distal tip 912). Additionally, the distalmost portion of distal tip 912 may be blunted to minimize trauma and damage to surrounding tissue during insertion. Notch 914 provides additional room for the proximal end of lead 100 having a rigid electrical connector to more easily be inserted when loading lead 100 in delivery system 200. Rails 916 overlap lead 100 and hold lead 100 flat when the lead is retracted and held within delivery system 200. In some implementations, the inner edge of rails 916 gradually widen as rails 916 advance toward distal tip 912.

FIG. 9D illustrates certain features applicable to a unitary insertion tip design.

In some implementations, insertion tip 900 may include a movable cover 918 configured to prevent the biological tissue from entering distal orifice 908 when insertion tip 900 pushes through the biological tissue. The moveable cover may move to facilitate advancement of component 904 into the patient.

It is contemplated that many of the other technologies disclosed herein can also be used with the unitary tip design. For example, insertion tip 900 may include a ramped portion 910 configured to facilitate advancement of the component into the patient in a particular direction and to allow the protruding electrodes 210, 212 to pass easier through the channel created within insertion tip 900.

In some implementations, delivery system 200 (FIG. 3) may include a dilator. In some implementations, insertion tips 304, 306, and/or insertion tip 900 may operate in conjunction with such a dilator. Use of a dilator may allow an initial incision to be smaller than it may otherwise be. The dilator may be directionally oriented to facilitate insertion of a component (e.g., an electrical lead) through the positioned dilator manually, and/or by other means. The dilator may comprise a mechanism that separates first and second insertion tips 304, 306. For example, relatively thin first and second insertion tips 304, 306 may be advanced through biological tissue. An actuator (e.g., a handle, and/or a device couple to the handle operated by the user) may insert a hollow, dilating wedge that separates first and second insertion tips 304, 306. The actuator (operated by the user) may advance a lead through the hollow dilator into the biological tissue. The dilator may also be used to separate the first and second insertion tips 304, 306 such that they lock into an open position. The dilator can then be removed and the lead advanced into the biological tissue.

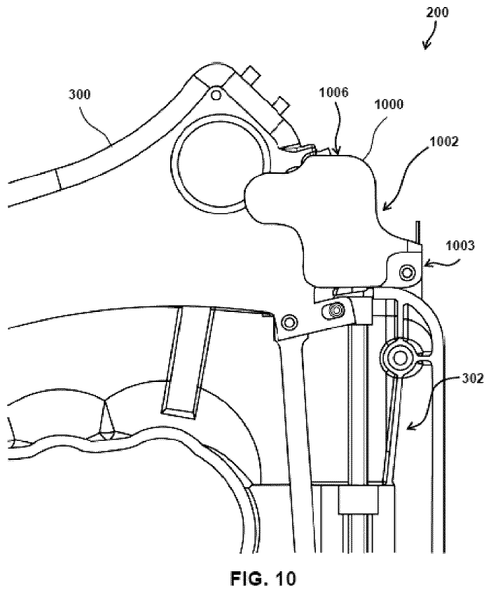

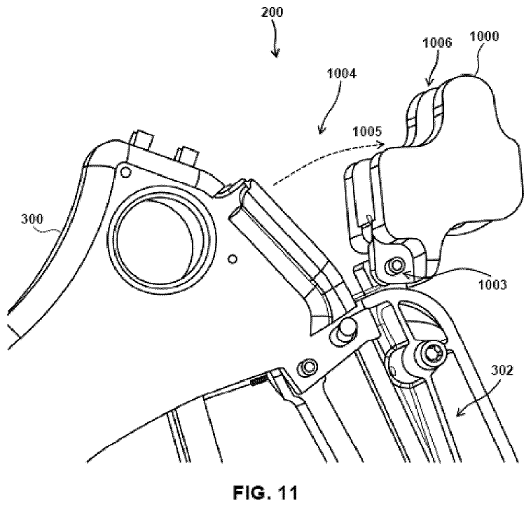

FIGS. 10 and 11 illustrate an exemplary lock 1000 that may be included in delivery system 200. A lock 1000 may be similar to and/or the same as lock 308 shown in FIG. 3. In some implementations, lock 1000 may be configured to be moved between an unlocked position that allows actuation of handle 300 (and in turn component advancer 302) by the operator and a locked position that prevents actuation, and prevents first insertion tip 304 (FIG. 7) and second insertion 306 tip (FIG. 7) from opening.

FIG. 10 illustrates lock 1000 in a locked position 1002. FIG. 11 illustrates lock 1000 in an unlocked position 1004. Lock 1000 may be coupled to handle 300 and/or component advancer 302 via a hinge 1003 and/or other coupling mechanisms. In some implementations, lock 1000 may be moved from locked position 1002 to unlocked position 1004, and vice versa, by rotating and/or otherwise moving an end 1006 of lock 1000 away from handle 300 (see, e.g., 1005 in FIG. 11). Lock 1000 may be moved from locked position 1002 to unlocked position 1004, and vice versa, by the operator with thumb pressure, trigger activation (button/lever, etc.) for example, and/or other movements. Additionally, the mechanism may also include a safety switch such that a trigger mechanism must be deployed prior to unlocking the lock with the operator's thumb.

When lock 1000 is engaged or in locked position 1002, lock 1000 may prevent an operator from inadvertently squeezing handle 300 to deploy the component. Lock 1000 may prevent the (1) spreading of the distal tips 304, 306, and/or (2) deployment of a component while delivery system 200 is being inserted through the intercostal muscles.

Lock 1000 may be configured such that deployment of the component may occur only when lock 1000 is disengaged (e.g., in the unlocked position 1004 shown in FIG. 11). Deployment may be prevented, for example, while an operator is using insertion tips 304, 306 of delivery system 200 to slide between planes of tissue in the intercostal space as pressure is applied to delivery system 200. Lock 1000 may be configured such that, only once system 200 is fully inserted into the patient can lock 1000 be moved so that handle 300 may be actuated to deliver the component through the spread (e.g., open) insertion tips 304, 306. It should be noted that the specific design of lock 1000 shown in FIGS. 10 and 11 is not intended to be limiting. Other locking mechanism designs are contemplated. For example, the lock 1000 may be designed so that lock 1000 must be fully unlocked to allow the handle 300 to be deployed. A partial unlocking of lock 1000 maintains the handle in the locked position as a safety mechanism. Furthermore, the lock 1000 may be configured such that any movement from its fully unlocked position will relock the handle 300.

Returning to FIG. 3, component advancer 302 may be configured to advance a component into a patient. The component may be an electrical lead (e.g., as described herein), and/or other components.

The component advancer 302 may be configured to removably engage a portion of the component, and/or to deliver the component into the patient through insertion tips 304 and 306. In some implementations, component advancer 302 and/or other components of system 200 may include leveraging components configured to provide a mechanical advantage or a mechanical disadvantage to an operator such that actuation of handle 300 by the operator makes advancing the component into the patient easier or more difficult. For example, the leveraging components may be configured such that a small and/or relatively light actuation pressure on handle 300 causes a large movement of a component (e.g., full deployment) from component advancer 302. Or, in contrast, the leveraging components may be configured such that a strong and/or relatively intense actuation pressure is required to deliver the component. In some implementations, the leveraging components may include levers, hinges, wedges, gears, and/or other leveraging components (e.g., as described herein). In some implementations, handle 300 may be advanced in order to build up torque onto component advancer 302, without moving the component. Once sufficient torque has built up within the component advancer, the mechanism triggers the release of the stored torque onto the component advancer, deploying the component.

In some implementations, component advancer 302 may include a rack and pinion system coupled to handle 300 and configured to grip the component such that actuation of handle 300 by the operator causes movement of the component via the rack and pinion system to advance the component into the patient. In some implementations, the rack and pinion system may be configured such that movement of handle 300 moves a single or dual rack including gears configured to engage and rotate a single pinion or multiple pinions that engage the component, so that when the single pinion or multiple pinions rotate, force is exerted on the component to advance the component into the patient.

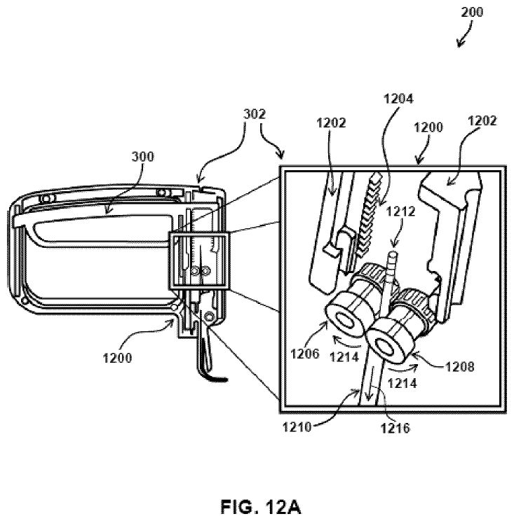

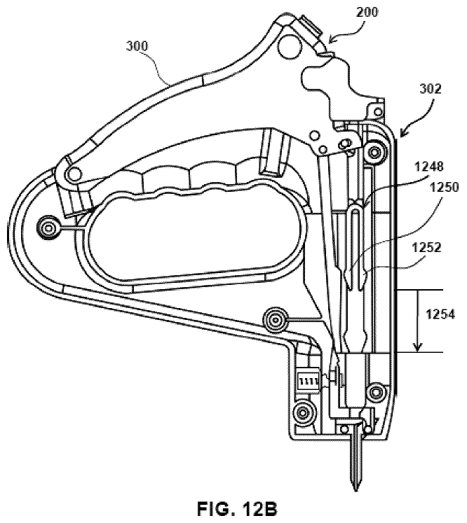

FIG. 12A illustrates an exemplary rack and pinion system 1200. Rack and pinion system may include rack(s) 1202 with gears 1204. Example system 1200 includes two pinions 1206, 1208. Pinions 1206 and 1208 may be configured to couple with a component 1210 (e.g., an electrical lead), at or near a distal end 1212 of component 1210, as shown in FIG. 12A. Rack and pinion system 1200 may be configured such that movement of handle 300 moves rack 1202 comprising gears 1204 configured to engage and rotate pinions 1206, 1208 that engage component 1210, so that when pinions 1206, 1208 rotate 1214, force is exerted 1216 on component 1210 to advance component 1210 into the patient.

In some implementations, responsive to handle 300 being actuated, a component (e.g., component 1210) may be gripped around a length of a body of the component, as shown in FIG. 12B. The body of the component may be gripped by two opposing portions 1250, 1252 of component advancer 302 that engage either side of the component, by two opposing portions that engage around an entire circumferential length of a portion of the body, and/or by other gripping mechanisms.

Once gripped, further actuation of handle 300 may force the two opposing portions within component advancer 302 to traverse toward a patient through delivery system 200. Because the component may be secured by these two opposing portions, the component may be pushed out of delivery system 200 and into the (e.g., anterior mediastinum) of the patient. By way of a non-limiting example, component advancer 302 may comprise a clamp 1248 having a first side 1250 and a second side 1252 configured to engage a portion of the component. Clamp 1248 may be coupled to handle 300 such that actuation of handle 300 by the operator may cause movement of the first side 1250 and second side 1252 of clamp 1248 to push on the portion of the component to advance the component into the patient. Upon advancing the component a fixed distance (e.g., distance 1254) into the patient, clamp 1248 may release the component. Other gripping mechanisms are also contemplated.

Returning to FIG. 3, in some implementations, component advancer 302 may include a pusher tube coupled with handle 300 such that actuation of handle 300 by the operator causes movement of the pusher tube to push on the portion of the component to advance the component into the patient. In some implementations, the pusher tube may be a hypo tube, and/or other tubes. In some implementations, the hypo tube may be stainless steel and/or be formed from other materials. However, these examples are not intended to be limiting. The pusher tube may be any tube that allows system 200 to function as described herein.

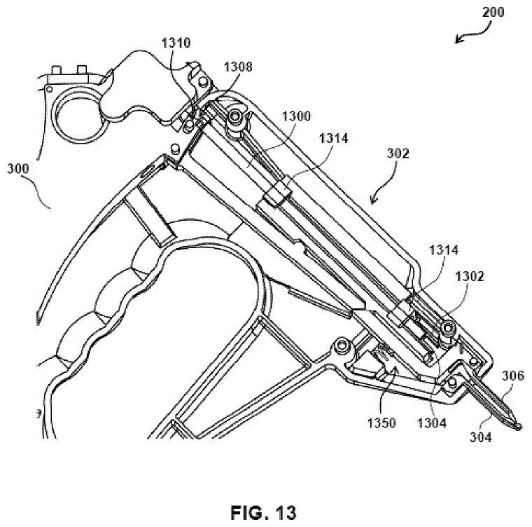

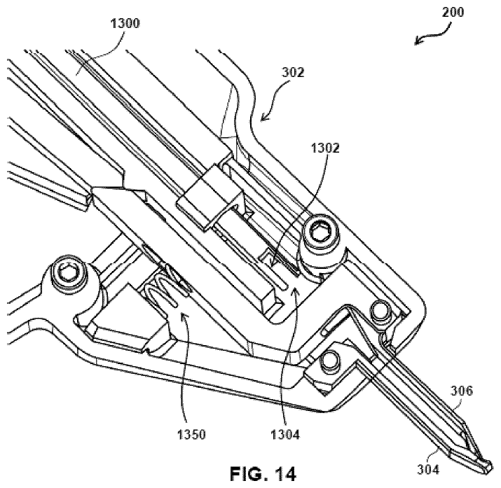

FIGS. 13 and 14 illustrate different views of an exemplary implementation of a component advancer 302 including a pusher tube 1300 coupled with handle 300. As shown in FIG. 13, in some implementations, pusher tube 1300 may include a notch 1302 having a shape complementary to a portion of a component and configured to maintain the component in a particular orientation so as to avoid rotation of the component within system 200. FIG. 13 shows notch 1302 formed in a distal end 1304 of pusher tube 1300 configured to mate and/or otherwise engage with an end of a distal portion of a component (not shown in FIG. 13) to be implanted. Pusher tube 1300 may be configured to push, advance, and/or otherwise propel a component toward and/or into a patient via notch 1302 responsive to actuation of handle 300.

In some implementations, the proximal end 1308 of pusher tube 1300 may be coupled to handle 300 via a joint 1310. Joint 1310 may be configured to translate articulation of handle 300 by an operator into movement of pusher tube 1300 toward a patient. Joint 1310 may include one or more of a pin, an orifice, a hinge, and/or other components. In some implementations, component advancer 302 may include one or more guide components 1314 configured to guide pusher tube 1300 toward the patient responsive to the motion translation by joint 1310. In some implementations, guide components 1314 may include sleeves, clamps, clips, elbow shaped guide components, and/or other guide components. Guide components 1314 may also add a tensioning feature to ensure the proper tactile feedback to the physician during deployment. For example, if there is too much resistance through guide components 1314, then the handle 300 will be too difficult to move. Additionally, if there is too little resistance through the guide components 1314, then the handle 300 will have little tension and may depress freely to some degree when delivery system 200 is inverted.

FIG. 14 provides an enlarged view of distal end 1304 of pusher tube 1300. As shown in FIG. 14, notch 1302 is configured with a rectangular shape. This rectangular shape is configured to mate with and/or otherwise engage a corresponding rectangular portion of a component (e.g., as described below). The rectangular shape is configured to maintain the component in a specific orientation. For example, responsive to a component engaging pusher tube 1300 via notch 1302, opposing (e.g., parallel in this example) surfaces, and/or the perpendicular (in this example) end surface of the rectangular shape of notch 1302 may be configured to prevent rotation of the component. This notch shape is not intended to be limiting. Notch 1302 may have any shape that allows it to engage a corresponding portion of a component and prevent rotation of the component as described herein. For example, in some implementations, pusher tube 1300 may include one or more coupling features (e.g., in addition to or instead of the notch) configured to engage the portion of the component and configured to maintain the component in a particular orientation so as to avoid rotation of the component within system 200. These coupling features may include, for example, mechanical pins on either side of the pusher tube 1300 configured to mate with and/or otherwise engage receptacle features on a corresponding portion of a component.

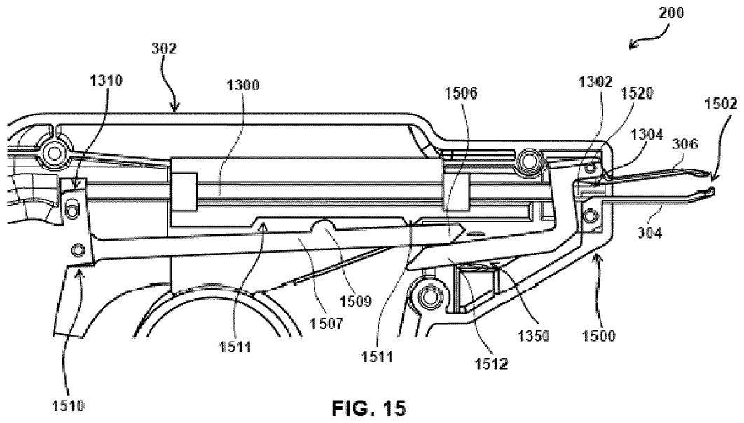

FIG. 15 illustrates insertion tips 304 and 306 in an open position 1502. FIG. 15 also illustrates pusher tube 1300 in an advanced position 1500, caused by actuation of handle 300 (not shown). Advanced position 1500 of pusher tube 1300 may be a position that is closer to insertion tips 304, 306 relative to the position of pusher tube 1300 shown in FIG. 14.

In some implementations, the component advancer 302 may include a wedge 1506 configured to move insertion tip 304 and/or 306 to the open position 1502. In some implementations, wedge 1506 may be configured to cause movement of the moveable insertion tip 306 and may or may not cause movement of insertion tip 304.

Wedge 1506 may be coupled to handle 300, for example, via a joint 1510 and/or other components. Joint 1510 may be configured to translate articulation of handle 300 by an operator into movement of the wedge 1506. Joint 1510 may include one or more of a pin, an orifice, a hinge, and/or other components. Wedge 1506 may be designed to include an elongated portion 1507 configured to extend from joint 1510 toward insertion tip 306. In some implementations, wedge 1506 may include a protrusion 1509 and/or other components configured to interact with corresponding parts 1511 of component advancer 302 to limit a travel distance of wedge 1506 toward insertion tip 306 and/or handle 300.

Wedge 1506 may also be slidably engaged with a portion 1512 of moveable insertion tip 306 such that actuation of handle 300 causes wedge 1506 to slide across portion 1512 of moveable insertion tip 306 in order to move moveable insertion tip 306 away from fixed insertion tip 304. For example, insertion tip 306 may be coupled to component advancer 302 via a hinge 1520. Wedge 1506 sliding across portion 1512 of moveable insertion tip 306 may cause moveable insertion tip to rotate about hinge 1520 to move moveable insertion tip 306 away from fixed insertion tip 304 and into open position 1502. In some implementations, moveable insertion tip 306 may be biased to a closed position. For example, a spring mechanism 1350 (also labeled in FIGS. 13 and 14) and/or other mechanisms may perform such biasing for insertion tip 306. Spring mechanism 1350 may force insertion tip 306 into the closed position until wedge 1506 is advanced across portion 1512, thereby separating insertion tip 306 from insertion tip 304.

In some implementations, as described above, first insertion tip 304 and second insertion tip 306 may be moveable. In some implementations, first insertion tip 304 and/or second insertion tip 306 may be biased to a closed position. For example, a spring mechanism similar to and/or the same as spring mechanism 1350 and/or other mechanisms may perform such biasing for first insertion tip 304 and/or second insertion tip 306. In such implementations, system 200 may comprise one or more wedges similar to and/or the same as wedge 1506 configured to cause movement of first and second insertion tips 304, 306. The one or more wedges may be coupled to handle 300 and slidably engaged with first and second insertion tips 304, 306 such that actuation of handle 300 may cause the one or more wedges to slide across one or more portions of first and second insertion tips 304, 306 to move first and second insertion tips 304, 306 away from each other.

In some implementations, system 200 may comprise a spring/lock mechanism or a rack and pinion system configured to engage and cause movement of moveable insertion tip 306. The spring/lock mechanism or the rack and pinion system may be configured to move moveable insertion tip 306 away from fixed insertion tip 304, for example. A spring lock design may include design elements that force the separation of insertion tips 304 and 306. One such example may include spring forces that remain locked in a compressed state until the component advancer or separating wedge activate a release trigger, thereby releasing the compressed spring force onto insertion tip 306, creating a separating force. These spring forces must be of sufficient magnitude to create the desired separation of tips 304 and 306 in the biological tissue. Alternatively, the spring compression may forceable close the insertion tips until the closing force is released by the actuator. Once released, the tips are then driven to a separating position by the advancement wedge mechanism, as described herein.

In some implementations, the component delivered by delivery system 200 (e.g., described above) may be an electrical lead for implantation in the patient. The lead may comprise a distal portion, one or more electrodes, a proximal portion, and/or other components. The distal portion may be configured to engage component advancer 302 of delivery system 200 (e.g., via notch 1302 shown in FIGS. 13 and 14). The distal portion may comprise the one or more electrodes. For example, the one or more electrodes may be coupled to the distal portion. The one or more electrodes may be configured to generate therapeutic energy for biological tissue of the patient. The therapeutic energy may be, for example, electrical pulses and/or other therapeutic energy. The biological tissue may be the heart (e.g., heart 118 shown in FIG. 1-FIG. 2C) and/or other biological tissue. The proximal portion may be coupled to the distal portion. The proximal portion may be configured to engage a controller when the lead is implanted in the patient. The controller may be configured to cause the one or more electrodes to generate the therapeutic energy, and/or perform other operations.

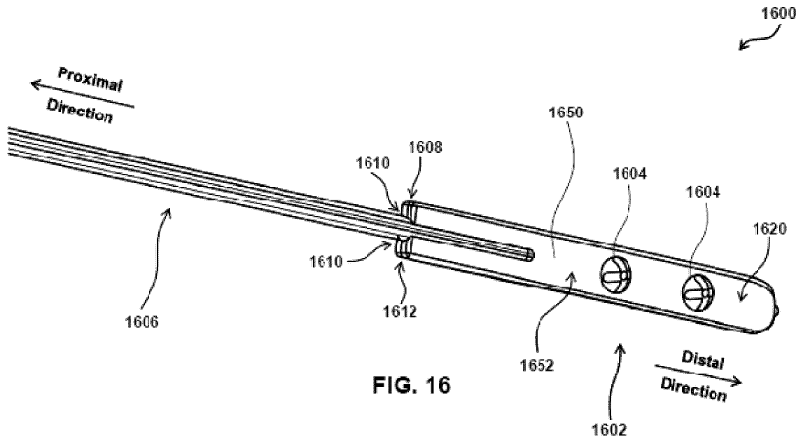

FIG. 16 illustrates an example implementation of an electrical lead 1600. Lead 1600 may comprise a distal portion 1602, one or more electrodes 1604, a proximal portion 1606, and/or other components. Distal portion 1602 may be configured to engage component advancer 302 of delivery system 200 (e.g., via notch 1302 shown in FIGS. 13 and 14). In some implementations, distal portion 1602 may comprise a proximal shoulder 1608. Proximal shoulder may be configured to engage component advancer 302 (e.g., via notch 1302 shown in FIGS. 13 and 14) such that lead 1600 is maintained in a particular orientation when lead 1600 is advanced into the patient. For example, in some implementations, proximal shoulder 1608 may comprise a flat surface 1610 (e.g., at a proximal end of distal portion 1602). In some implementations, proximal shoulder 1608 may comprise a rectangular shape 1612. Flat surface 1610 and/or rectangular shape 1612 may be configured to correspond to a (e.g., rectangular) shape of notch 1302 shown in FIGS. 13 and 14. In some implementations, transition surfaces between flat surface 1610 and other portions of distal portion 1602 may be chamfered, rounded, tapered, and/or have other shapes.

In some implementations, proximal shoulder 1608 may include one or more coupling features configured to engage component advancer 302 to maintain the lead in a particular orientation so as to avoid rotation of the lead when the lead is advanced into the patient. In some implementations, these coupling features may include receptacles for pins included in pusher tube 1300, clips, clamps, sockets, and/or other coupling features.

In some implementations, proximal shoulder 1608 may comprise the same material used for other portions of distal portion 1602. In some implementations, proximal shoulder may comprise a more rigid material, and the material may become less rigid across proximal shoulder 1608 toward distal end 1620 of distal portion 1602.

In some implementations, proximal shoulder 1608 may function as a fixation feature configured to make removal of lead 1600 from a patient (and/or notch 1302) more difficult. For example, when lead 1600 is deployed into the patient, lead 1600 may enter the patient led by a distal end 1620 of the distal portion 1602. However, retracting lead 1600 from the patient may require the retraction to overcome the flat and/or rectangular profile of flat surface 1610 and/or rectangular shape 1612, which should be met with more resistance. In some implementations, delivery system 200 (FIG. 3) may include a removal device comprising a sheath with a tapered proximal end that can be inserted over lead 1600 so that when it is desirable to intentionally remove lead 1600, the flat and/or rectangular profile of shoulder 1608 does not interact with the tissue on the way out.

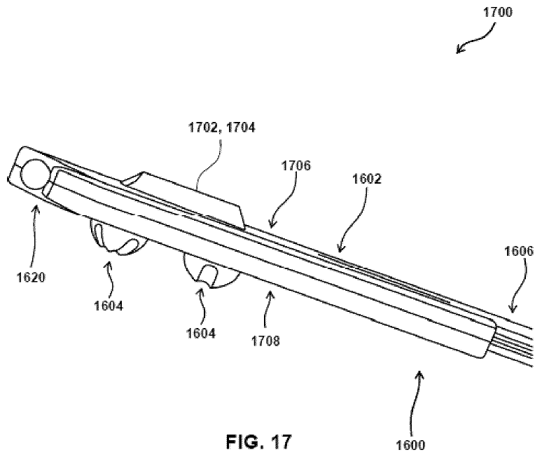

FIG. 17 illustrates another example implementation 1700 of electrical lead 1600. In some implementations, as shown in FIG. 17, distal portion 1602 may include one or more alignment features 1702 configured to engage delivery system 200 (FIG. 3) in a specific orientation. For example, alignment features 1702 of lead 1600 may include a rib 1704 and/or other alignment features configured to engage a groove in a channel (e.g., channel 500 shown in FIG. 5) of insertion tip 304 and/or 306 (FIG. 5). Rib 1704 may be on an opposite side 1706 of the lead 1600 relative to a side 1708 with electrodes 1604, for example. These features may enhance the guidance of lead 1600 through channel 500, facilitate alignment of lead 1600 in channel 500 (e.g., such that electrodes 1604 are oriented in a specific direction in tips 304, 306), prevent lead 1600 from exiting tips 304, 306 to one side or the other (as opposed to exiting out ends 404, 406 shown in FIG. 4), and/or have other functionality.

In some implementations, rib 1704 may be sized to be just large enough to fit within the groove in the channel 500. This may prevent the lead from moving within the closed insertion tips 304, 306 while the insertion tips are pushed through the intercostal muscle tissue. Additionally, rib 1704 may prevent an operator from pulling lead 1600 too far up into delivery system 200 (FIG. 3) when loading delivery system 200 with a lead (e.g., as described below). This may provide a clinical benefit, as described above, and/or have other advantages.

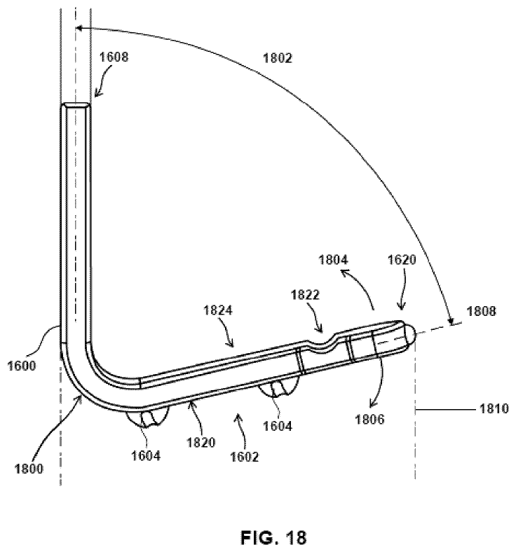

FIG. 18 illustrates distal portion 1602 of lead 1600 bent 1800 in a predetermined direction 1804. In some implementations, distal portion 1602 may be pre-formed to bend in predetermined direction 1804. The pre-forming may shape set distal portion 1602 with a specific shape, for example. In the example, shown in FIG. 18, the specific shape may form an acute angle 1802 between ends 1620, 1608 of distal portion 1602. The pre-forming may occur before lead 1600 is loaded into delivery system 200 (FIG. 3), for example. In some implementations, distal portion 1602 may comprise a shape memory material configured to bend in predetermined direction 1804 when lead 1600 exits delivery system 200. The shape memory material may comprise nitinol, a shape memory polymer, and/or other shape memory materials, for example. The preforming may include shape setting the shape memory material in the specific shape before lead 1600 is loaded into delivery system 200.

Distal portion 1602 may be configured to move in an opposite direction 1806, from a first position 1808 to a second position 1810 when lead 1600 enters the patient. In some implementations, first position 1808 may comprise an acute angle 1802 shape. In some implementations, the first position may comprise a ninety degree angle 1802 shape, or an obtuse angle 1802 shape. In some implementations, the second position may comprise a ninety degree angle 1802 shape, or an obtuse angle 1802 shape. Distal portion 1602 may be configured to move from first position 1808 to second position 1810 responsive to the shape memory material being heated to body temperature or by removal of an internal wire stylet, for example. In some implementations, this movement may cause an electrode side of distal portion 1602 to push electrodes 1604 into tissues toward a patient's heart, rather than retract away from such tissue and the heart. This may enhance electrical connectivity and/or accurately delivering therapeutic energy toward the patient's heart, for example.

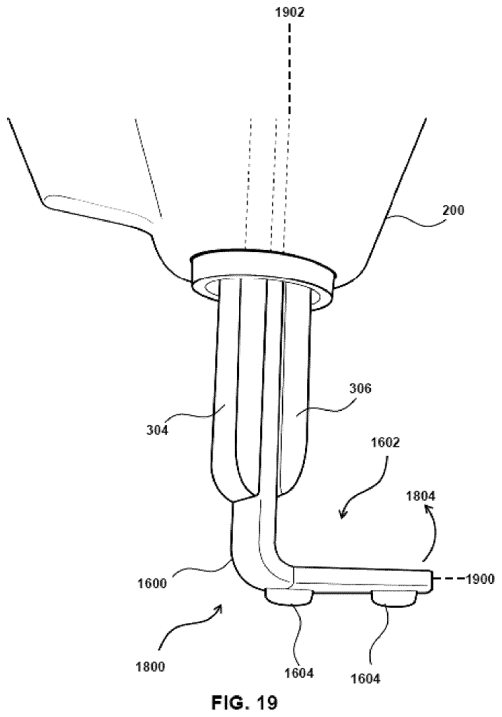

FIG. 19 illustrates distal portion 1602 bending 1800 in the predetermined direction 1804 when lead 1600 exits delivery system 200. In some implementations, as shown in FIG. 19, the predetermined direction may comprise a lateral and/or transverse direction 1900 relative to an orientation 1902 of insertion tips 304 and/or 306, a sternum of the patient, and/or other reference points in delivery system 200 and/or in the patient.

Any of the designs discussed herein can have a predetermined shape that can result in a lead moving in a predetermined direction or having a predetermined shape when the lead exits delivery system 200. In some cases, the direction can be determined or facilitated by the design of the delivery system (e.g., implementations herein where leads are directed utilizing ramps). In other implementations, the direction or shape may be determined by the design of the lead itself (e.g., a lead with a preformed shape that is forced to be held straight when within delivery system 200 but that assumes the preformed shape again upon exiting the delivery system). The present disclosure also contemplates leads being delivered over a stylet which can similarly hold a lead with a preformed shape until the stylet is removed and the lead reverts back to its preformed shape.

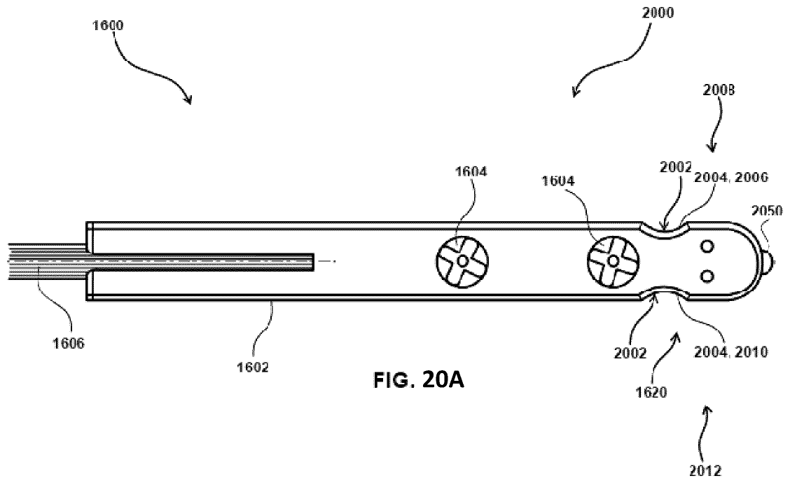

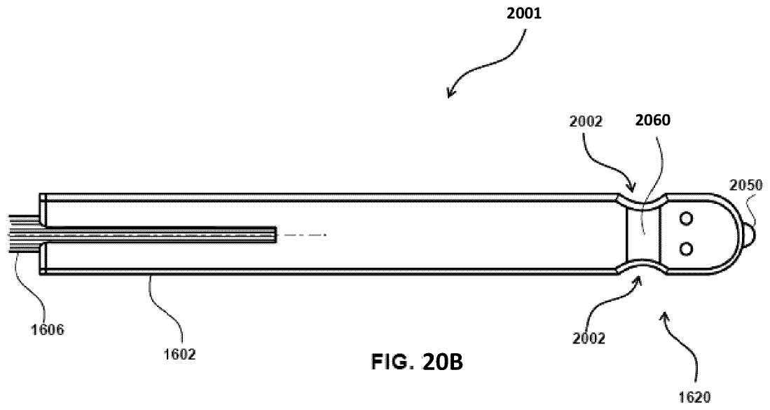

FIGS. 20A and 20B illustrate implementations 2000 and 2001 of distal portion 1602 of lead 1600. In some implementations, distal portion 1602 may include distal end 1620 and distal end 1620 may include a flexible portion 2002 so as to allow distal end 1620 to change course when encountering sufficient resistance traveling through the biological tissue of the patient. In some implementations, distal end 1620 may be at least partially paddle shaped, and/or have other shapes. The paddle shape may allow more surface area of distal end 1620 to contact tissue so the tissue is then exerting more force back on distal end 1620, making distal end 1620 bend and flex via flexible portion 2002. In some implementations, flexible portion 2002 may comprise a material that flexes more easily relative to a material of another area of distal portion 1602. For example, flexible portion 2002 may comprise a different polymer relative to other areas of distal portion 1602, a metal, and/or other materials.

In some implementations, flexible portion 2002 may comprise one or more cutouts 2004. The one or more cutouts 2004 may comprise one or more areas having a reduced cross section compared to other areas of distal portion 1602. The one or more cutouts 2004 may be formed by tapering portions of distal portion 1602, removing material from distal portion 1602, and/or forming cutouts 2004 in other ways. The cutouts may increase the flexibility of distal end 1620, increase a surface area of distal end 1620 to drive distal end 1620 in a desired direction, and/or have other purposes. Cutouts 2004 may reduce a cross-sectional area of distal end 1620, making distal end 1620 more flexible, and making distal end 1620 easier to deflect. Without such cutouts, for example, distal end 1620 may be too rigid or strong, and drive lead 1600 in a direction that causes undesirable damage to organs and/or tissues within the anterior mediastinum (e.g., the pericardium or heart).

In some implementations, the one or more areas having the reduced cross section (e.g., the cutouts) include a first area (e.g., cutout) 2006 on a first side 2008 of distal end 1620. The one or more areas having the reduced cross section (e.g., cutouts) may include first area 2006 on first side 2008 of distal end 1620 and a second area 2010 on a second, opposite side 2012 of distal end 1620. This may appear to form a neck and/or other features in distal portion 1602, for example.

In some implementations, as shown in FIG. 20B, the one or more areas having the reduced cross section may include one or more cutouts 2060 that surround distal end 1620. Referring back to FIG. 18, in some implementations, distal portion 1602 may have a surface 1820 that includes one or more electrodes 1604, and a cut out 1822 in a surface 1824 of distal end 1620 opposite surface 1820 with one or more electrodes 1604. This positioning of cutout 1822 may promote a bias of distal end 1620 back toward proximal shoulder 1608 (FIG. 16) of lead 1600. In some implementations, cutout 1822 may create a bias (depending upon the location of cutout 2060) acutely in direction 1804 or obtusely in direction 1806. Similarly, alternative cutouts 2060 may be inserted to bias distal end 1620 in other directions.

Returning to FIGS. 20A and 20B, in some implementations, flexible portion 2002 may be configured to cause distal end 1620 to be biased to change course in a particular direction. Distal end 1620 may change course in a particular direction responsive to encountering resistance from biological tissue in a patient, for example. In some implementations, biasing distal end 1620 to change course in a particular direction may comprise biasing distal end 1620 to maintain electrodes 1604 on a side of distal portion 1602 that faces the heart of the patient. For example, distal end 1620 may be configured to flex or bend to push through a resistive portion of biological material without twisting or rotating to change an orientation of electrodes 1604.

In some implementations, distal portion 1602 may include a distal tip 2050 located at a tip of distal end 1620. Distal tip 2050 may be smaller than distal end 1620. Distal tip 2050 may be more rigid compared to other portions of distal end 2050. For example, distal tip 2050 may be formed from metal (e.g., that is harder than other metal/polymers used for other portions of distal end 1620), hardened metal, a ceramic, a hard plastic, and/or other materials. In some implementations, distal tip 2050 may be blunt, but configured to push through biological tissue such as the endothoracic fascia, and/or other biological tissue. In some implementations, distal tip 2050 may have a hemispherical shape, and/or other blunt shapes that may still push through biological tissue.

In some implementations, distal tip 2050 may be configured to function as an electrode (e.g., as described herein). This may facilitate multiple sense/pace vectors being programmed and used without the need to reposition electrical lead 1600. For example, once the electrical lead 1600 is positioned, electrical connections can be made to the electrodes 1604 and cardiac pacing and sensing evaluations performed. If unsatisfactory pacing and/or sensing performance is noted, an electrical connection may be switched from one of the electrodes 1604 to the distal electrode 2050. Cardiac pacing and/or sensing parameter testing may then be retested between one of the electrodes 1604 and the distal electrode 2050. Any combination of two electrodes can be envisioned for the delivery of electrical therapy and sensing of cardiac activity, including the combination of multiple electrodes to create one virtual electrode, then used in conjunction with a remaining electrode or electrode pairing. Additionally, electrode pairing may be selectively switched for electrical therapy delivery vs. physiological sensing.

Returning to FIG. 16, in some implementations, at least a portion of distal portion 1602 of lead 1600 may comprise two parallel planar surfaces 1650. One or more electrodes 1604 may be located on one of the parallel planar surfaces, for example. Parallel planar surfaces 1650 may comprise elongated, substantially flat surfaces, for example. (Only one parallel planar surface 1650 is shown in FIG. 16. The other parallel planar surface 1650 may be located on a side of distal portion 1602 opposite electrodes 1604, for example.) In some implementations, at least a portion 1652 of distal portion 1602 of lead 1600 may comprise a rectangular prism including the two parallel planar surfaces 1650.

Because the proximal end of the distal portion 1602 may be positioned within the intercostal muscle tissue (while the distal end of the distal portion 1602 resides in the mediastinum), the elongated, substantially flat surfaces of proximal end of the distal portion 1602 may reduce and/or prevent rotation of distal portion 1602 within the muscle tissue and within the mediastinum. In contrast, a tubular element may be free to rotate. In some implementations, distal portion 1602 may include one or more elements configured to engage and/or catch tissue to prevent rotation, prevent egress and/or further ingress of distal portion 1602, and/or prevent other movement. Examples of these elements may include tines, hooks, and/or other elements that are likely to catch and/or hold onto biological tissue. In some implementations, the bending of distal portion 1602 (e.g., as described above related to FIG. 18) may also function to resist rotation and/or other unintended movement of distal portion 1602 in a patient. Distal portion 1602 may also be designed with multiple segments, with small separating gaps between each segment, designed to increase stability within the tissue, increase the force required for lead retraction or to promote tissue ingrowth within the distal portion 1602.

In accordance with certain disclosed embodiments, the present disclosure contemplates systems and methods that include placing a lead having both defibrillation and cardiac pacing electrodes at an extravascular location within a patient. The extravascular location can be in a mediastinum of the patient, and specifically may be in a region of the cardiac notch or on or near the inner surface of a patient's intercostal muscle. As such, some placement methods can also include inserting the lead through an intercostal space associated with the cardiac notch of the patient.

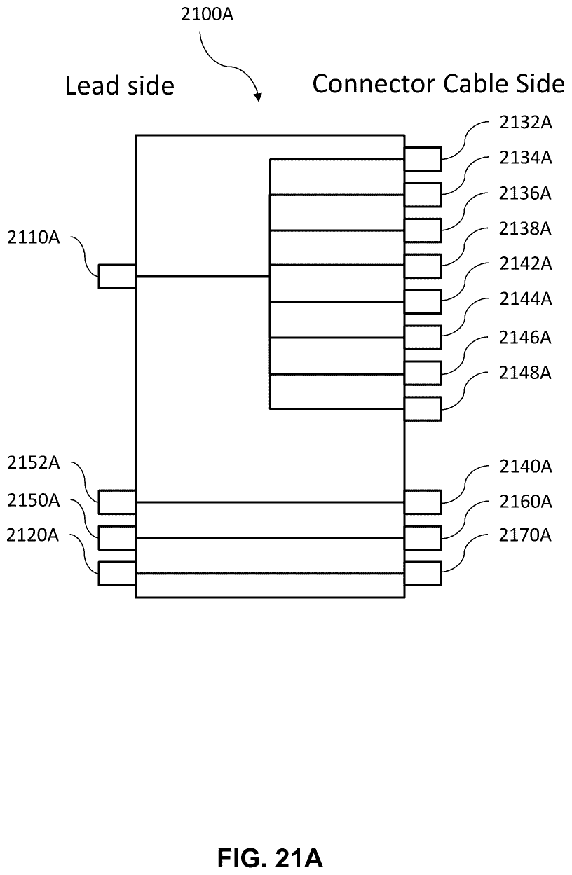

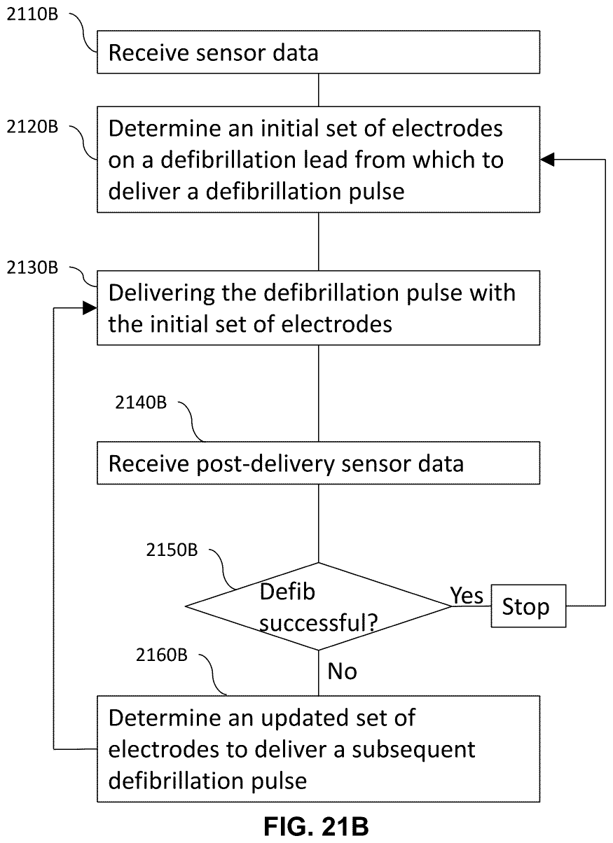

FIG. 21A depicts an exemplary junction box 2100 that can facilitate connections between the lead and its control and sensing systems. Such connections can be provided to provide pass through between the various pacing and defibrillation electrodes on the lead and the various input connections on the defibrillation source, one example being to an implantable ICD with a DF-4 connector. In the example implementation shown, the previously described leads can have corresponding junction box connections (2132A, 2134A, 2136A, 2138A, 2142A, 2144A, 2146A, 2148A) on the lead side of the junction box. The electrodes can be connected via a single lead 2110A (e.g., a multi-wire cable) at the connector cable side of the junction box. There can also be dedicated connections 2150A, 2152A for a pacing anode and cathode. The junction box can also have a lead side connection 2120A to the coil body itself (e.g., to a housing or grounding mesh) and corresponding SVC connection 2170A. Cathode connection 2152A can be connected to a corresponding “tip” connection 2140A. Anode connection 2150A can be connected to a corresponding “ring” connection 2160A.

An exemplary method utilizing the leads described above is shown in the flowchart of FIG. 21B. In implementations where defibrillation electrodes are disposed on different locations of a lead, as described above, defibrillation pulses will propagate in different directions. In such implementations, the electrodes can also provide sensing information allowing determination of which defibrillation electrodes are directed at the heart in a manner to optimize defibrillation. With such a determination, the defibrillation pulses can be delivered through the optimal electrodes.

One exemplary method can include, at 2110B, receiving sensor data at a sensor (e.g., any disclosed electrode or other separate sensor), where the sensor data can be representative of electrical signals (e.g., from a heartbeat). At 2120B, an algorithm can determine, based on the sensor data, an initial set of electrodes on a defibrillation lead including more than two defibrillation electrodes, from which to deliver a defibrillation pulse. The initial electrode set can be one estimated to be most directed toward the heart and thereby most appropriate for defibrillation (for example, based on determining relative strengths of the signals detected by different sensing electrodes). At 2130B, a defibrillation pulse can be delivered with the initial set of electrodes. At 2140B, post-delivery sensor data can be received, such as by the sensor(s) described above. At 2150B, a determination can be made, based at least on the post-delivery sensor data whether the defibrillation pulse successfully defibrillated the patient. At 2160B, if necessary, an updated set of electrodes which to deliver a subsequent defibrillation pulse can be determined, with the process optionally repeating starting at 2130B with the delivery of the subsequent defibrillation pulse.

In step 2150B, the determination as to whether defibrillation was successful may include receiving signals representative of the current heart rhythm and comparing to an expected or desired heart rhythm that would be reflective of a successful defibrillation. In step 2160B, determining a new set of electrodes may include, for example, switching to some electrodes on the opposite side of the lead. The determination may also result in using a different set of electrodes on the same side of the lead. In fact, any combination of defibrillation electrodes on the lead, or in combination with electrodes located off of the lead (for example, on the housing of an associated pulse generator) may be utilized, including reversing the electrical polarity of the defibrillation shock. The process of delivering defibrillation energy and selecting different electrode pairings can repeat, cycling through different combinations, until a successful defibrillation is detected. Again referring to step 2150B, once a defibrillation configuration is determined that successfully defibrillates the heart, the system can retain that configuration so that it can be used for the first defibrillation delivery during a subsequent episode with the patient, thereby increasing the likelihood of successful defibrillation with the first delivered shock for future events.

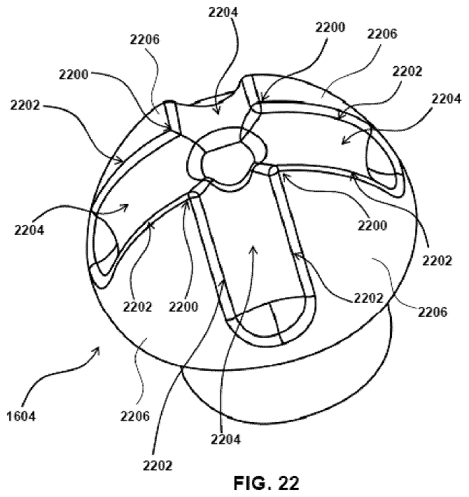

FIG. 22 illustrates an example of an electrode 1604. In some implementations, an electrode 1604 may be formed from a conductive metal and/or other materials. Electrodes 1604 may be configured to couple with distal portion 1602 of lead 1600, proximal portion 1606 (e.g., wiring configured to conduct an electrical signal from a controller) of lead 1600, and/or other portions of lead 1600. In some implementations, distal portion 1602 may comprise a rigid material, with an area of distal portion 1602 around electrodes 1604 comprising a relatively softer material. One or more electrodes 1604 may protrude from distal portion 1602 of lead 1600 (e.g., as shown in FIG. 16). Electrodes 1604 may be configured to provide electrical stimulation to the patient or to sense electrical or other physiologic activity from the patient (e.g., as described above). In some implementations, one or more electrodes 1604 may include one or both of corners 2200 and edges 2202 configured to enhance a current density in one or more electrodes 1604. In some implementations, at least one of the electrodes 1604 may comprise one or more channels 2204 on a surface 2206 of the electrode 1604. In some implementations, at least one of the one or more electrodes 1604 may comprise two intersecting channels 2204 on surface 2206 of the electrode 1604. In some implementations, the channels 2204 may be configured to increase a surface area of an electrode 1604 that may come into contact with biological tissue of a patient. Other channel designs are contemplated.

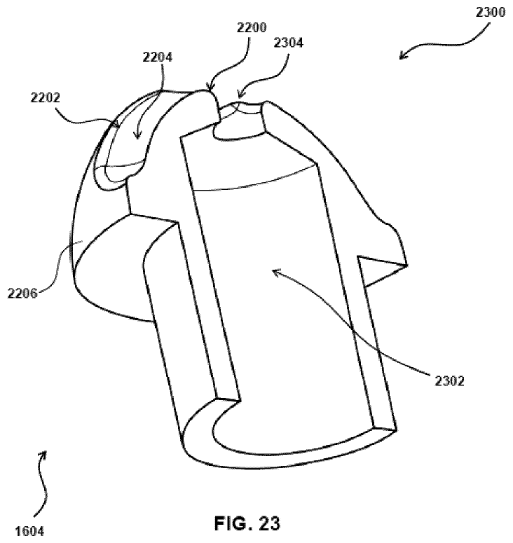

FIG. 23 illustrates a cross section 2300 of example electrode 1604. In some implementations, as shown in FIG. 23, at least one of the one or more electrodes 1604 may be at least partially hollow 2302. In such implementations, an electrode 1604 may include a hole 2304 configured to allow the ingress of fluid. In some implementations, an electrode 1604 may include a conductive mesh (not shown in FIG. 23) within hollow area 2302. The conductive mesh may be formed by conductive wiring, a porous sheet of conductive material, and/or other conductive meshes electrically coupled to electrode 1604. In some implementations, an electrode 1604 may include electrically coupled scaffolding within hollow area 2302. The scaffolding may be formed by one or more conductive beams and/or members placed in and/or across hollow area 2302, and/or other scaffolding.

These and/or other features of electrodes 1604 may be configured to increase a surface area and/or current density of an electrode 1604. For example, channels in electrodes 1604 may expose more surface area of an electrode 1604, and/or create edges and corners that increase current density, without increasing a size (e.g., the diameter) of an electrode 1604. The corners, hollow areas, conductive mesh, and/or scaffolding may function in a similar way.

In some implementations, an anti-inflammatory agent may be incorporated by coating or other means to electrode 1604. For example, a steroid material may be included in hollow area 2302 to reduce the patient's tissue inflammatory response.

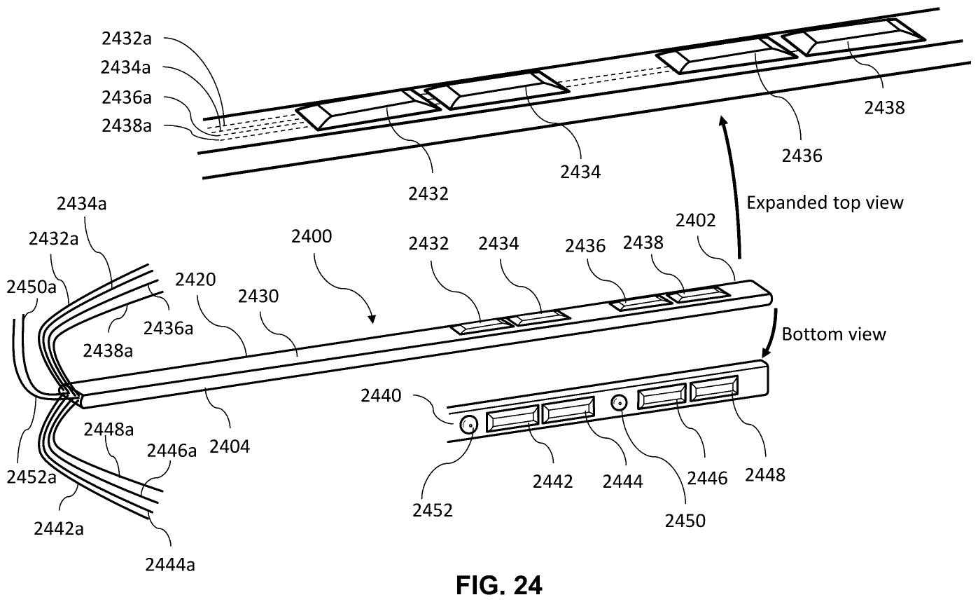

FIG. 24 illustrates an embodiment of a lead 2400 with parallel planar surfaces that include one or more electrodes. This electrical lead (or simply “lead”) for implantation in a patient is shown as having a distal portion 2402 (e.g., a portion deployed in a patient) and a proximal portion 2404. The distal portion can include one or more electrodes that are configured to generate therapeutic energy for biological tissue of a patient. The proximal portion can be coupled to the distal portion and configured to engage a controller that can be configured to cause the one or more electrodes to generate therapeutic energy.

At least a portion of the lead (e.g., the distal portion) may include two parallel planar surfaces that can form a rectangular prism. Various embodiments of the leads described herein can thus provide a distal portion configured for extravascular implantation. For example, these planar surfaces are well-suited for implantation near and/or along a patient's sternum. As used herein, the term “rectangular prism” refers to a lead having rectangular sides and/or cross section. Some sides/cross-sections may be square, as such is a type of rectangle. Also, a “rectangular prism” allows for small deviations from being perfectly rectangular. For example, edges may be rounded to prevent damage to patient tissues and some rectangular faces may have a slight degree of curvature (e.g., less than 30°).

The distal portion of the lead may include defibrillation electrodes or cardiac pacing electrodes. In some embodiments, the electrodes on the lead may include both defibrillation electrodes and cardiac pacing electrodes. One embodiment, depicted in FIG. 24, shows a lead body 2420 with a top side 2430, which may include electrodes 2432, 2434, 2436, 2438. Also shown as an inset is part of the bottom side 2440 of the lead (which would normally be obscured by the perspective view). The bottom side can have a similar, or identical, set of electrodes (2442, 2444, 2446, 2448). In the embodiments described herein, particularly those referencing FIGS. 24-27, electrodes are may described with reference to a particular “side” of a lead. However, it is contemplated that electrodes can be configured to provide directional stimulation from any side of the lead body. For example, rather than having electrodes present on the top side and the bottom side of a directional lead, there may be electrodes present on a top side and a left side of the directional lead. Accordingly, no particular combination, disposition, or shape of the disclosed electrodes should be considered essential to the present disclosure, other embodiments not specifically described are contemplated.

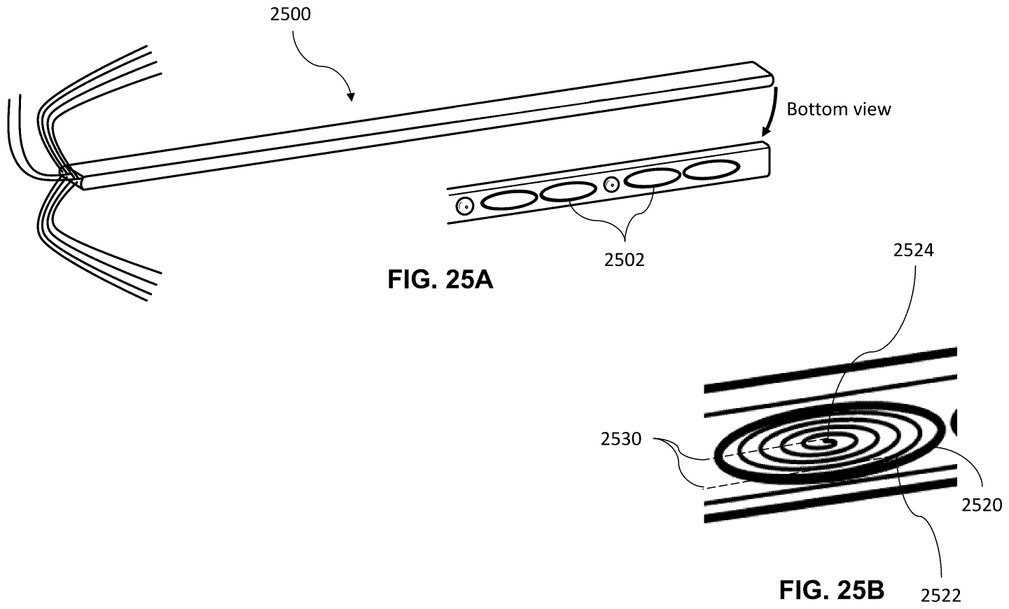

As shown in FIG. 24, the electrodes can be thin metallic plates (e.g., stainless steel, copper, other conductive materials, etc.) of a generally planar shape. The thin metallic plates can be rectangular (as shown in FIG. 24) but may also be elliptical (as shown in FIG. 25A). The panel electrodes may have rounded corners or edges to avoid damaging patient tissue. Certain embodiments of the thin metallic plates can be on one or both of the two parallel planar surfaces.

The embodiment of FIG. 24 depicts defibrillation panel electrodes along with a pacing anode 2450 and pacing cathode 2452. Although the embodiments depicted in the figures include pacing electrodes only on the bottom of the lead, it is contemplated that the lead may alternatively include pacing electrodes on either or both sides of the lead. In addition, the location of the anode and cathode may be reversed or moved to different locations on the lead. Additionally, multiple pacing anodes 2450 or pacing cathodes 2452 may be included on the same side of the lead.

While the embodiment of FIG. 24 depicts four top defibrillation electrodes and four bottom defibrillation electrodes, it is contemplated that various other arrangements and placements may be utilized, for example, two defibrillation electrodes on top and two defibrillation electrodes on bottom, etc. Also, it is contemplated that any of the corresponding top and bottom defibrillation electrodes may be connected, thereby delivering directional electrical energy simultaneously away from the top side and the bottom side of the lead body (e.g., electrodes 2432 and 2442 may be connected or formed as a single conductive element that extends through the lead body).

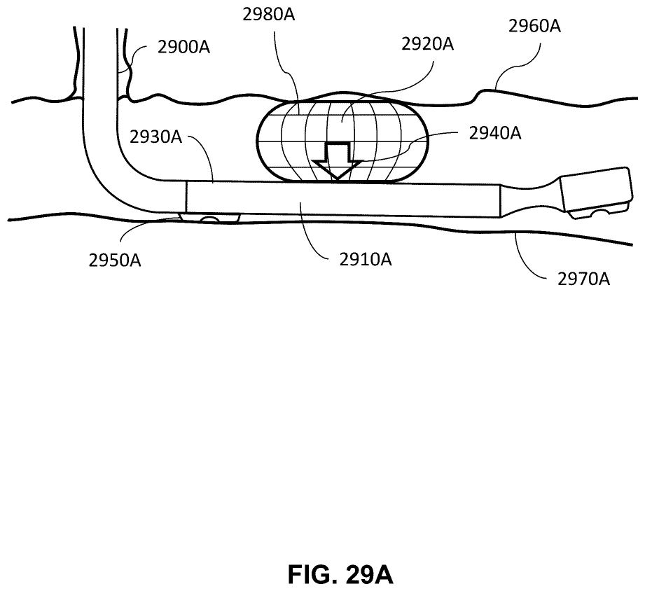

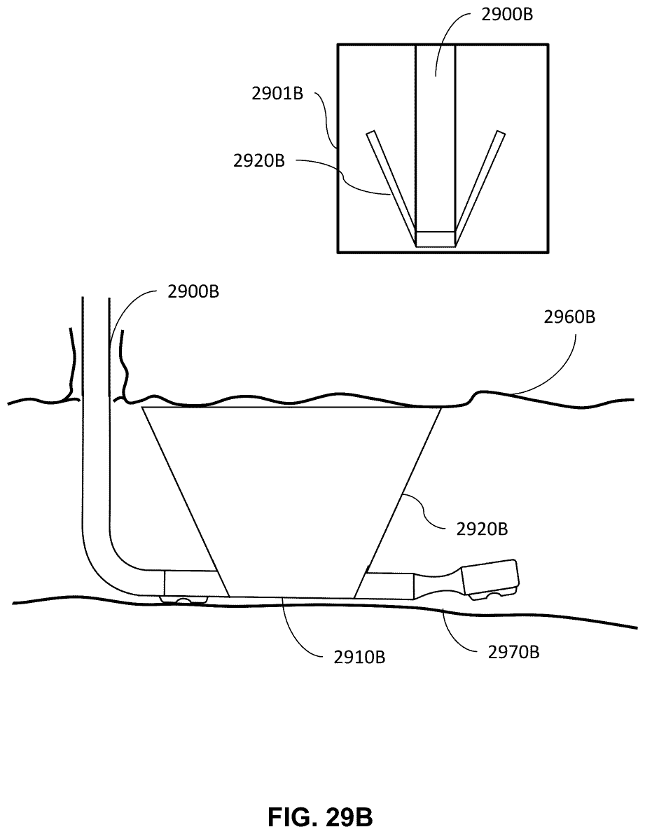

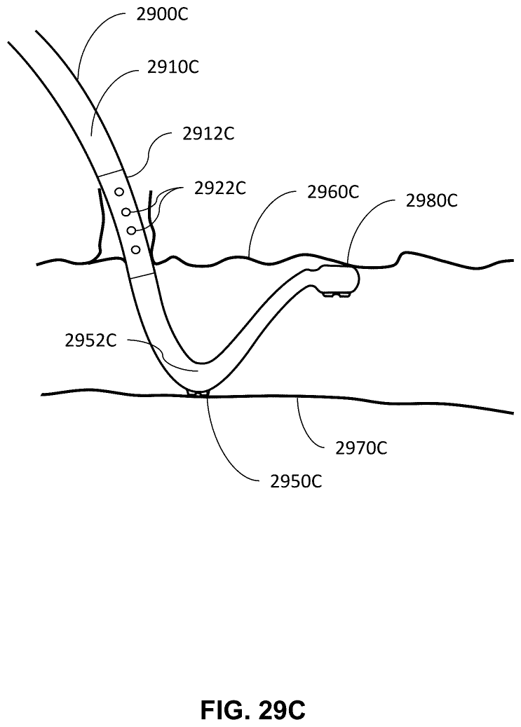

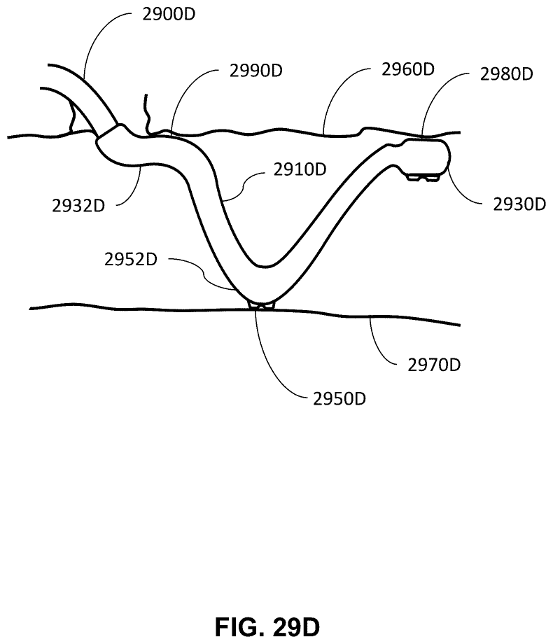

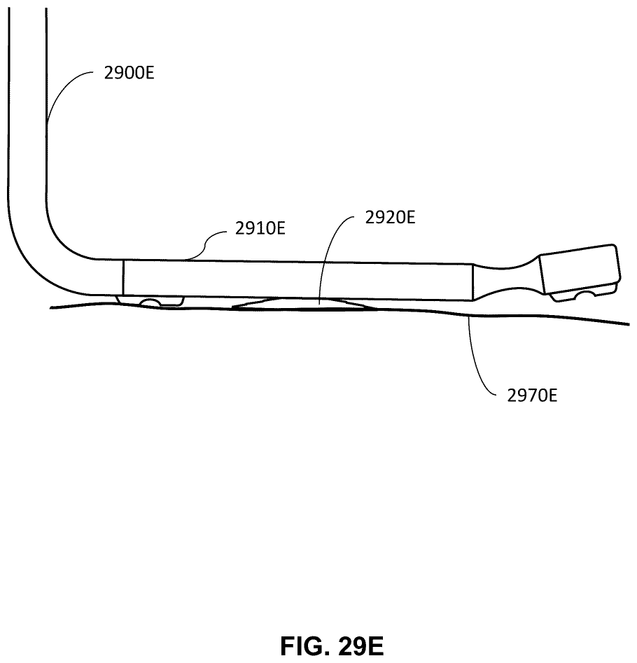

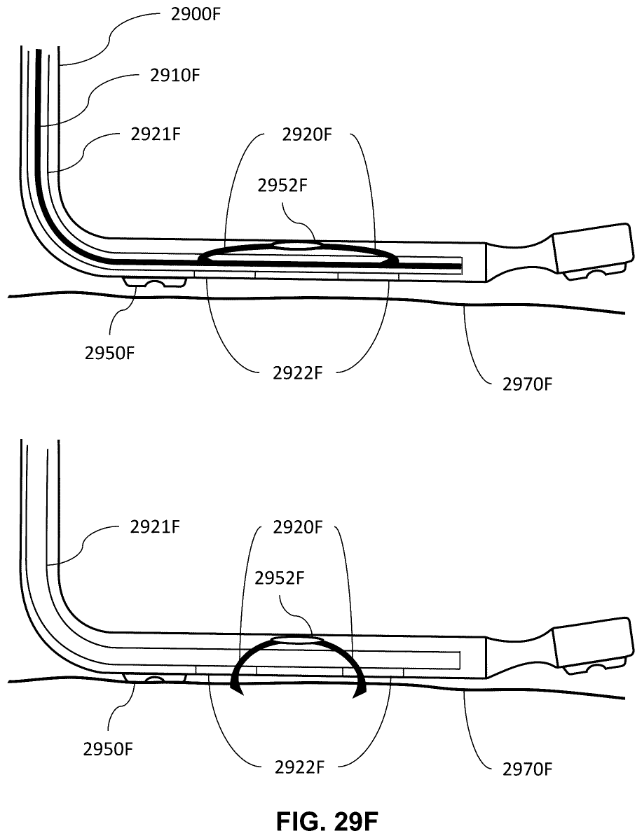

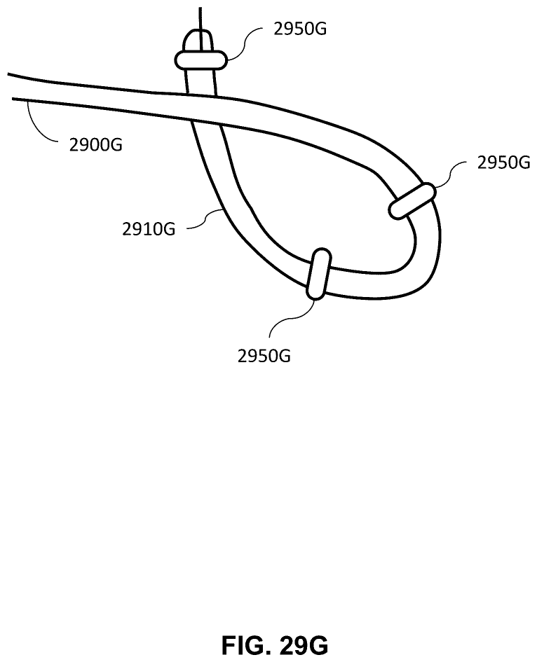

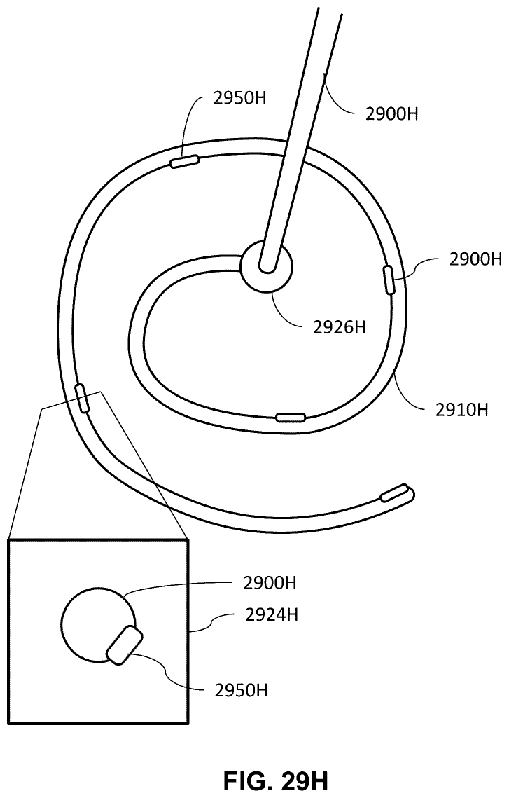

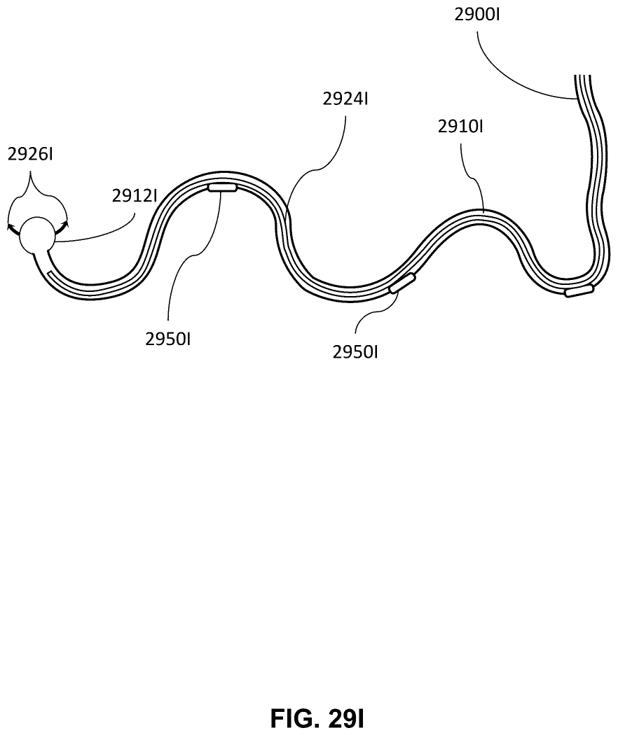

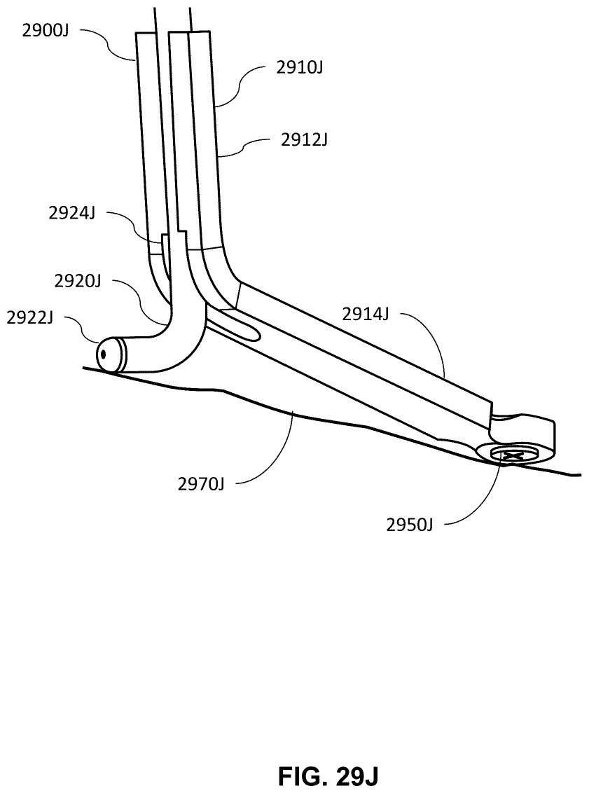

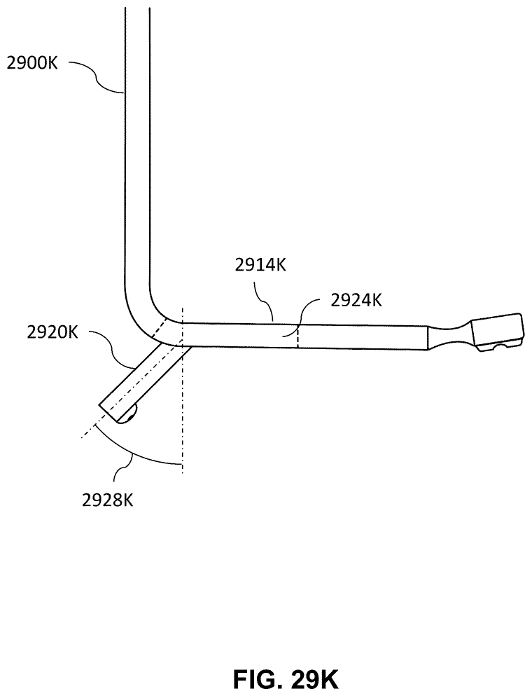

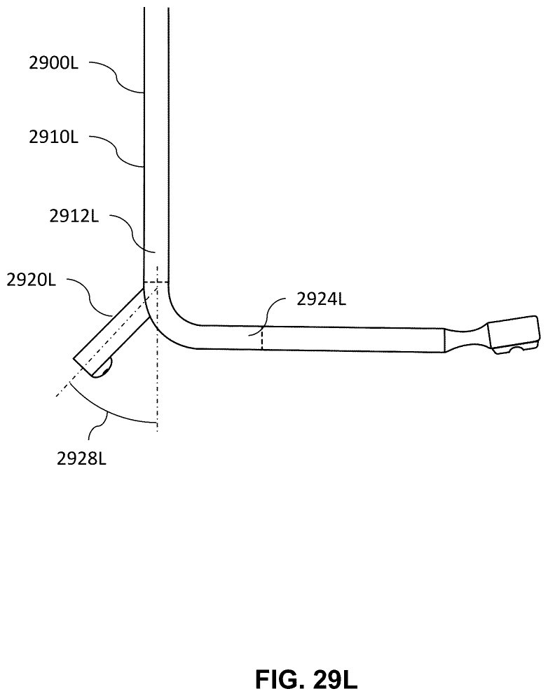

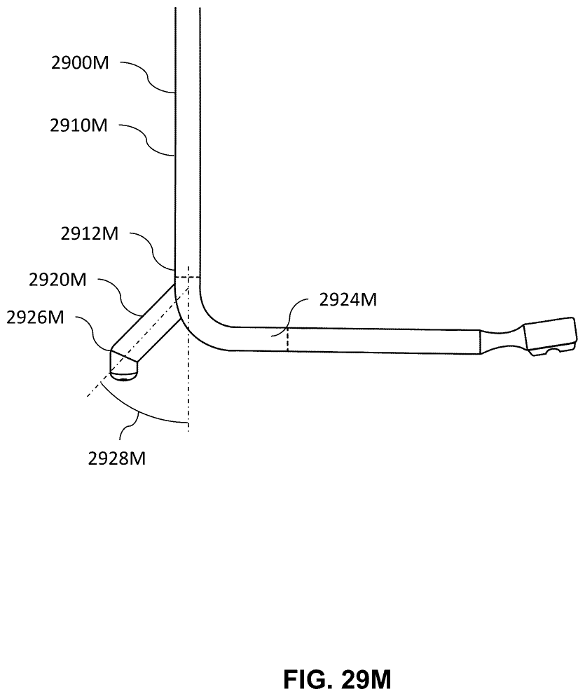

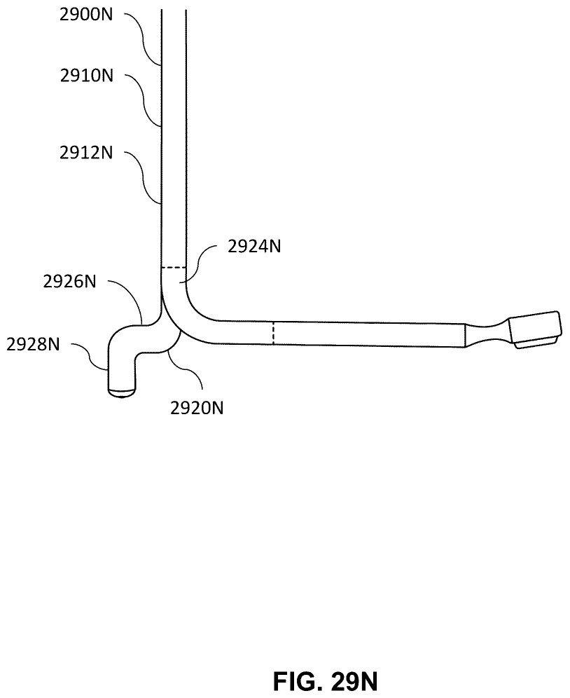

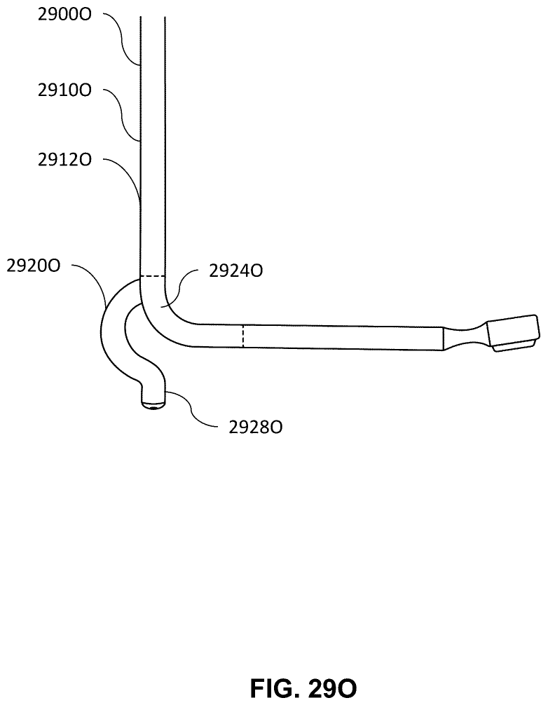

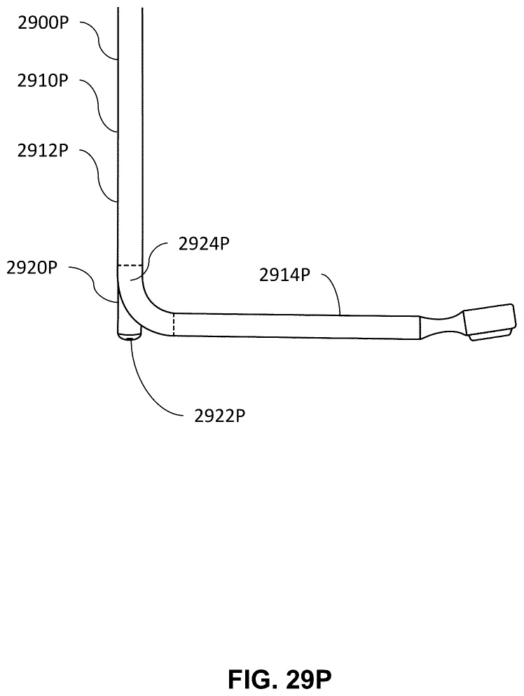

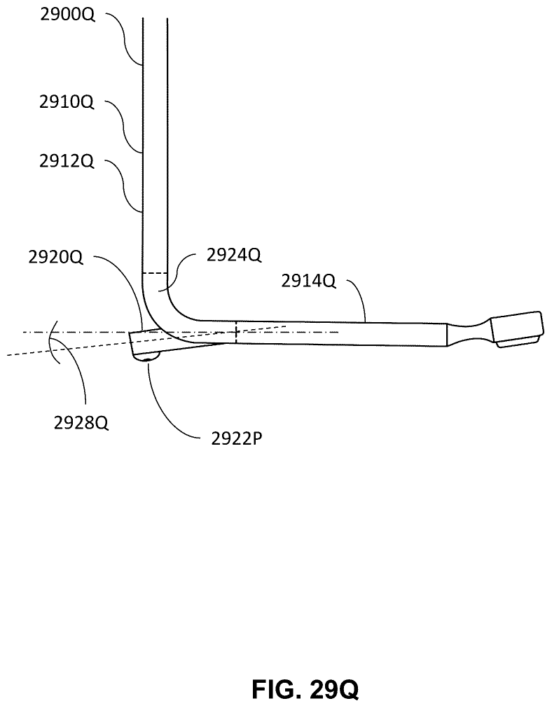

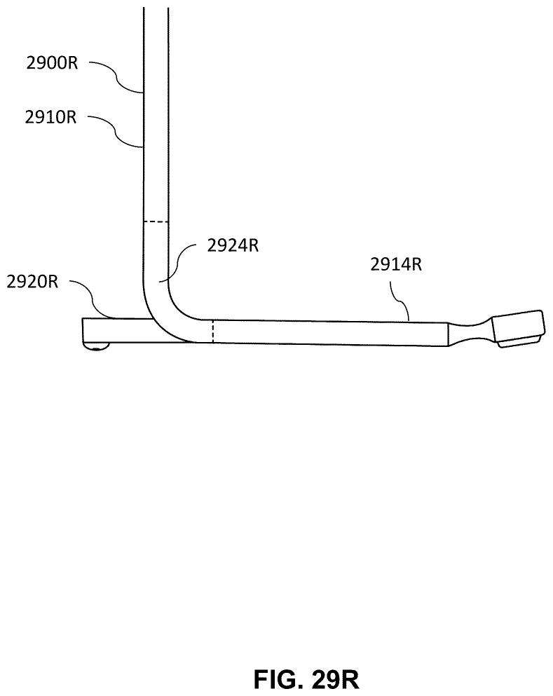

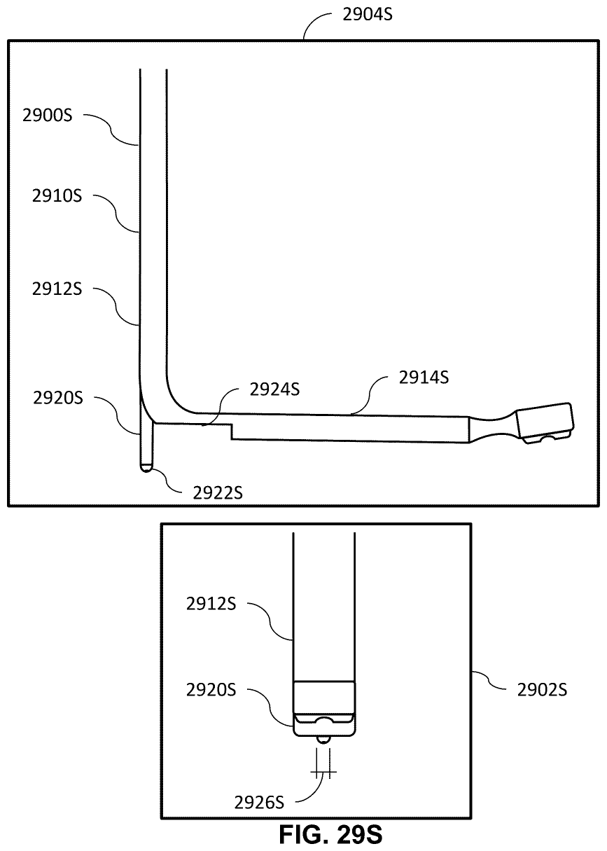

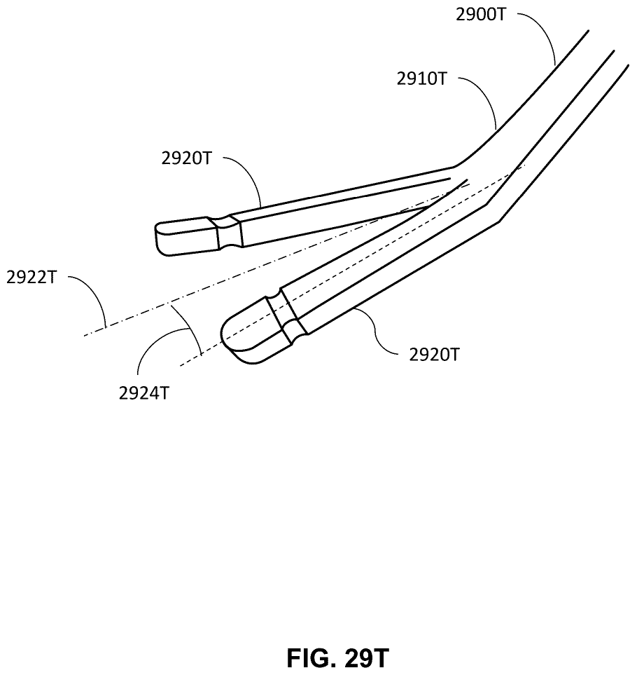

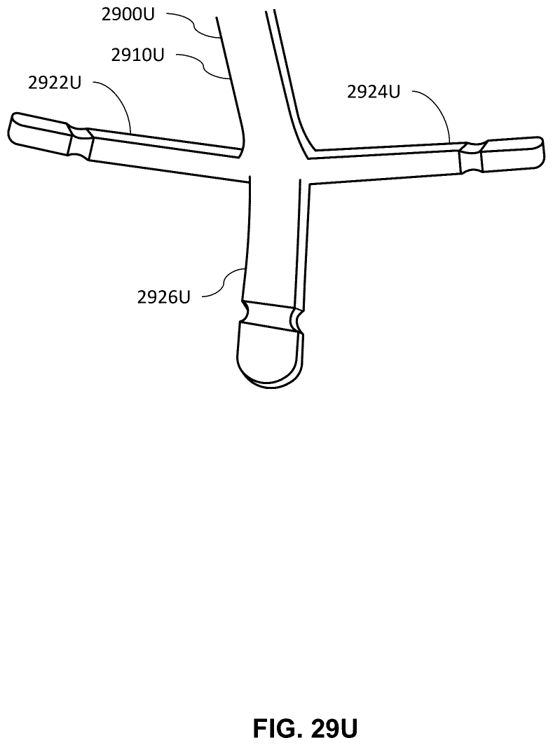

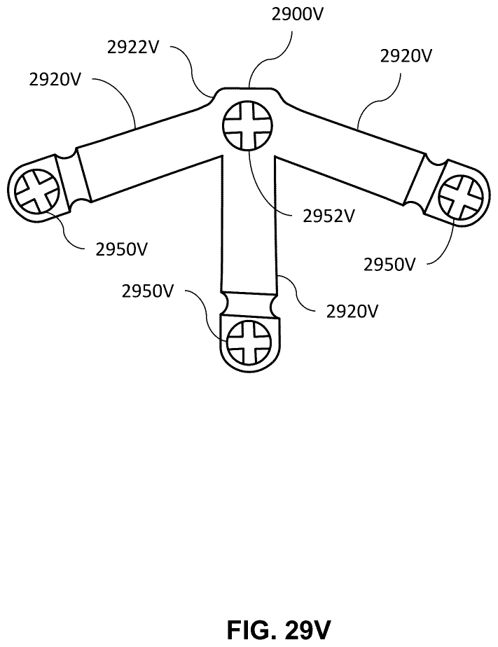

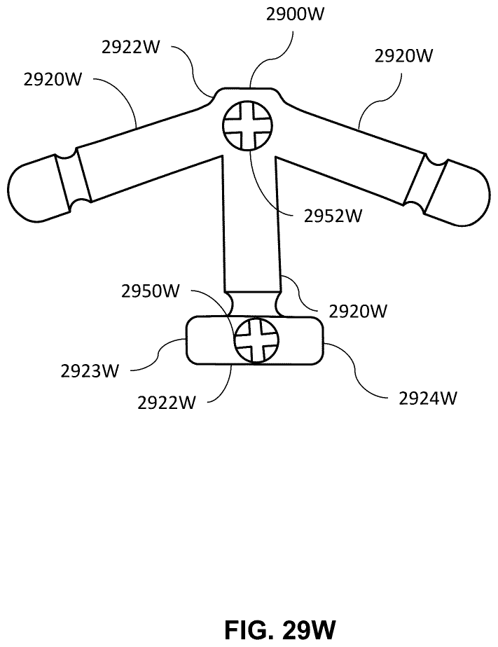

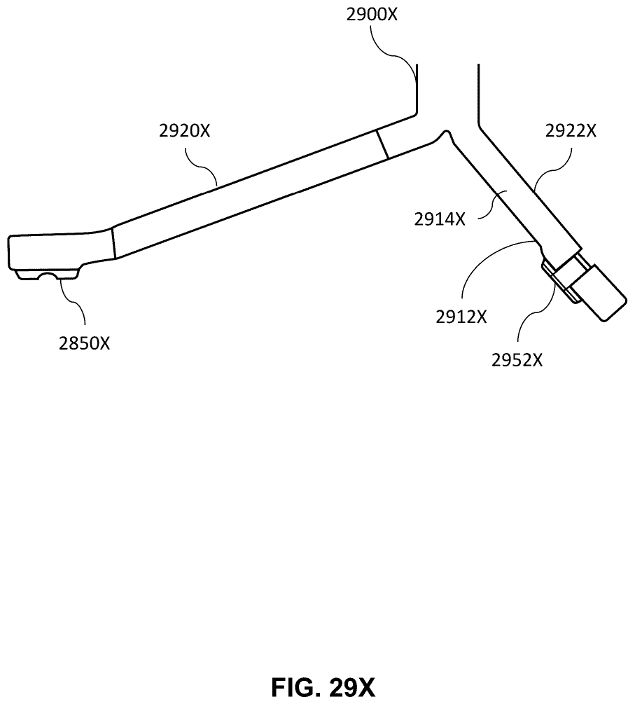

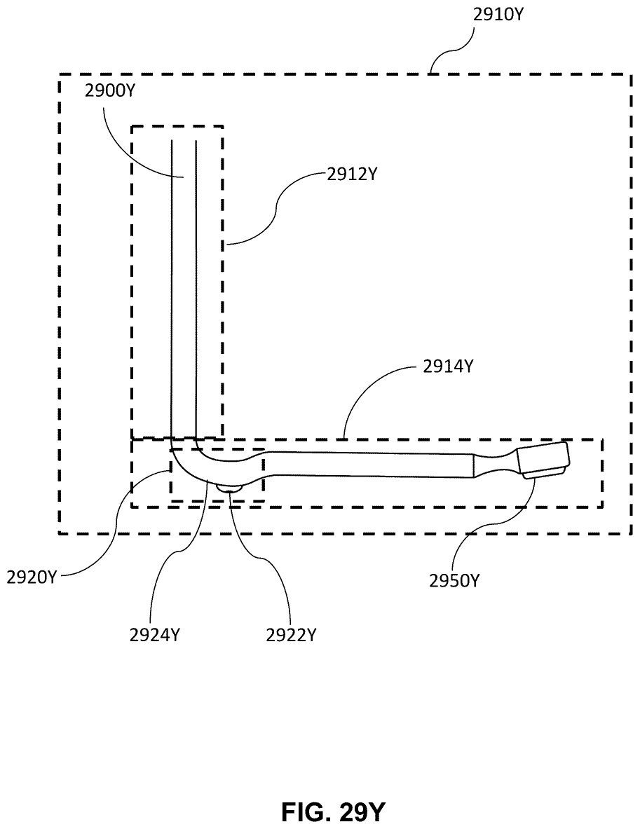

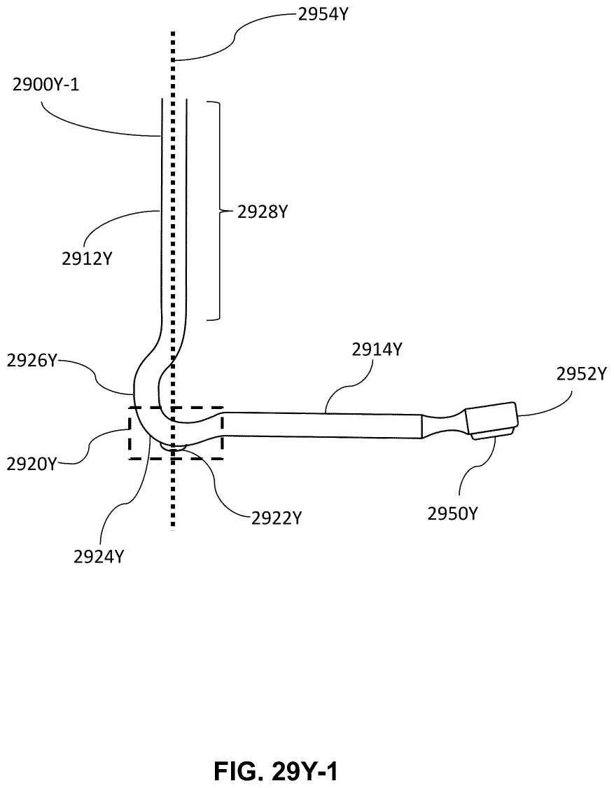

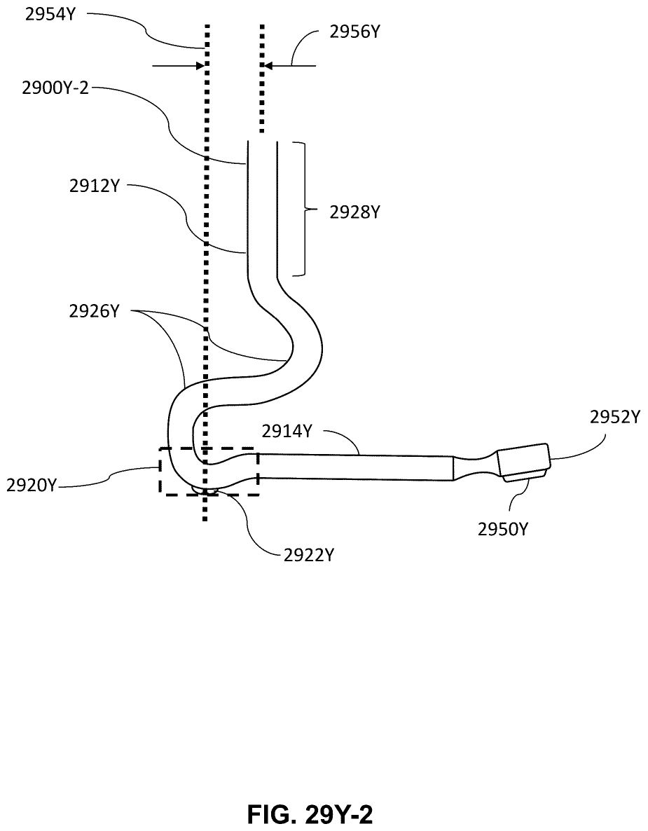

FIG. 24 also depicts leads wires (2432a, 2434a, 2436a, 2438a, 2442a, 2444a, 2446a, 2448a, 2450a, 2452a) that extend through or along the lead body and connect to their respective electrodes. The expanded top view illustrates the lead wires (2432a, 2434a, 2436a, 2438a) for the electrodes (2432, 2434, 2436, 2438) on the top of the lead. The lead wires can conduct defibrillation and pacing pulses and/or sensing signals to and/or from a connected pulse generator or computer that controls or processes signals. Similar to other embodiments described herein, the illustrated defibrillation electrodes can be energized in any combination to provide specific defibrillation vectors for delivering defibrillation pulses. Such energization can include varying the current through the defibrillation electrodes and thereby varying the defibrillation energy delivered to the heart. Aspects of such functionality are further described with reference to FIGS. 29A-29Y-2. Furthermore, multiple or all electrodes may be electrically tied together within the lead body such that only one lead wire emerges at the distal portion 2404. In some embodiments, the pacing cathode and anode are independently routed to the distal portion of the lead along with one defibrillation lead wire that is connected to all of the defibrillation electrodes. Alternatively, the pacing cathode may be independent; however, the pacing anode and defibrillation electrodes are electrically tied together within the lead body. In some instances, the defibrillation electrodes can act as the pacing anode for cardiac sensing and pacing therapies, while also serving as the defibrillation electrodes during defibrillation energy delivery. Additionally, redundant wires may be placed to ensure electrical connection with the various electrodes in the even that one wire is compromised.

While the depicted components (e.g., directional lead, lead body, electrodes, anode, cathode, etc.) can be designed to various dimensions, in an exemplary implementation, the lead body may have a width of approximately 5 mm and a thickness of approximately 2 mm, with panel electrodes being approximately 20 mm in length by 5 mm in width. Also, the pacing anodes and/or cathodes can have an approximately a 2-5 mm diameter. As used herein, the term “approximately,” when describing dimensions, means that small deviations are permitted such as typical manufacturing tolerances but may also include variations such as within 30% of stated dimensions.

The embodiments described herein are not intended to be limited to two opposite sides of a planar lead body. The teachings can apply similarly to a lead body that is round, with electrodes located at different angles around the circumference of the lead body.

FIG. 25A illustrates an embodiment of a lead 2500 with elliptical electrodes 2502. Such elliptical electrodes can be similar in many respects to the rectangular thin metallic plate electrodes described above but can have the benefit of providing a different current distribution to the patient than rectangular electrodes.

FIG. 25B illustrates an embodiment similar to the embodiment described with reference to FIG. 25A but instead of the electrodes being planar (e.g., a continuous sheet or plate) the defibrillation electrodes may be constructed as elliptical spiral coils 2520. Such spiral electrodes can have electrical current passed along the conductor in a spiral pattern. The conductors forming the spiral may have cross-sections that are round (e.g., wire), rectangular (e.g., flat), etc. The dimensions of the overall spiral can be similar to those described above with regard to the planar electrodes of FIG. 24. The configuration of the spiral can be such that there is a sufficient spacing (e.g., approximately 0.05 mm) to allow for flexibility which eases the delivery of the lead as compared to rigid panels. It is contemplated that the spiral can be constructed such that most of the area of the electrode is occupied by conductor, though in some implementations, the central portion may not be fully covered or may be covered in a looser spiral to manufacturing constraints. In some embodiments, the surface area of the spiral coil can be greater than 50%, 60 to 70%, 80 to 90%, or greater than 95% of the surface area enclosed by the largest perimeter of the spiral.

As shown in the magnified inset, spiral electrodes may have an inner termination 2522 and an outer termination 2524. The inner and outer terminations can be connected to corresponding connecting lead wires 2530 and such lead wires may extend through the lead body similarly to the configuration described with respect to FIG. 24. Pairs of leads (i.e., a lead for the inner termination and a lead for the outer termination) may be braided to reduce electrical interference. However, in some implementations, there may be a single lead connected to either the inner termination or the outer termination of the spiral. In such implementations, only the patient tissue acts as a return for the delivered current.

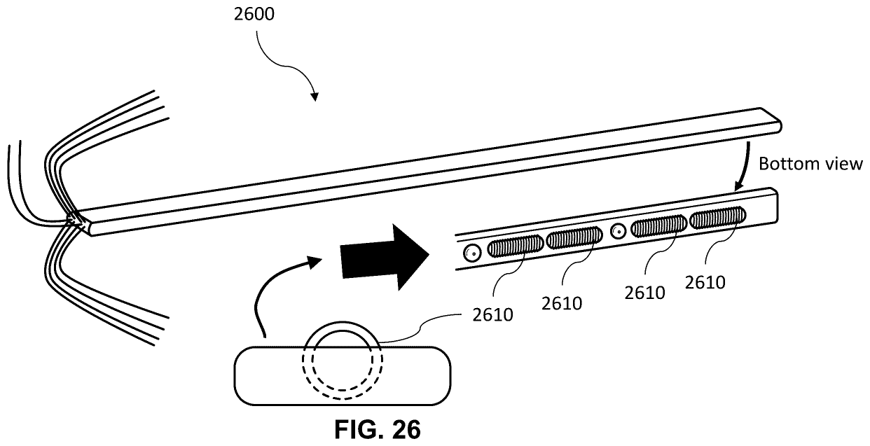

FIG. 26 illustrates an embodiment of a lead 2600 that has embedded electrodes 2610. Such embedded electrodes 2610 can be similar to previous embodiments in that they provide directional stimulation. To provide this directional stimulation, electrical energy from the embedded electrodes may be partially blocked by the insulating lead body. As shown in the depicted embodiment, embedded electrodes can be partially embedded in the portion of the distal portion of the lead having the two planar parallel surfaces. In this way, the partially embedded electrodes can have an embedded portion and an exposed portion.

In the embodiment of FIG. 26, the embedded electrodes are shown as helical coils that are oriented in the longitudinal direction (i.e., along the lengthwise direction of the lead body). The inset of FIG. 26 shows a simplified end view of the lead body with a portion of the embedded electrode being outside the lead body and the remainder of the embedded electrode being inside the lead body (as indicated by the dashed lines). As can be seen, the portion of the embedded electrode outside the lead body can thus have a similar surface area to the previously described planar electrodes. However, due to the helical shape of the embedded electrode, the portion that is extending from the lead body can have a greater vertical extent (i.e., can bulge outward) as compared to a thin metallic plate electrode and thus increase the available surface area.

The electrodes depicted in FIG. 26 are configured such that the exposed portion is on only one of the two planar parallel surfaces. However, it is contemplated that in other embodiments the electrodes may have portions that extend from more than one face. For example, were the electrode larger in diameter and/or shifted downward in the inset, there could be portions extending from both of the two planar parallel surfaces. In this way, the embedded electrode can provide directional stimulation, but in multiple directions, similar to embodiments where there may be top and bottom electrodes (e.g., in FIG. 24). In some embodiments, the degree of embeddedness can vary. For example, the exposed portion can include at least 25%, 50%, 75%, etc. of the partially embedded electrode.

As shown, the embedded electrode can be a circular helical coil (i.e., as if wrapped around a cylindrical object), however, other embodiments can have the embedded electrode be an elliptical helical coil (i.e., as if the object around which the wire was wrapped had an elliptical rather than circular cross-section). Yet other embodiments can have the embedded electrode be a solid electrode having a circular, elliptical, or rectangular cross-section. Some elliptical or rectangular embodiments can beneficially provide greater surface area while keeping the thickness of the coil (e.g., in the semimajor direction or in a thinner direction) at a minimum to reduce the overall thickness of the directional lead.

Some embodiments of partially embedded electrodes can include additional structural feature(s) to increase surface area beyond that provided by their cross-section. Examples of additional structural features can include conductive mesh. The conductive mesh may be formed by conductive wiring, a porous sheet of conductive material, and/or other conductive meshes electrically coupled to partially embedded electrode. These and/or other features of partially embedded electrodes may be configured to increase a surface area and/or current density of an electrode. For example, channels in partially embedded electrodes may expose more surface area, and/or create ridges, edges, and corners that increase current density, without increasing a size (e.g., the diameter) of an electrode. Implementations having such corners, hollow areas, conductive mesh, and/or scaffolding may function in a similar way.

Other embodiments of the partially embedded electrode can include an additional structural feature to increase current density beyond that provided by its cross-section and may also include a feature to increase current density at particular location(s). For example, as described above, ridges, edges, and corners may also have the effect of increasing current density due to charge accumulation. Other embodiments that may have increased surface area and/or current density can include electrodes with surfaces that have been treated by a sputtering process to create conductive microstructures or coatings that impart a texture to the electrode surface.

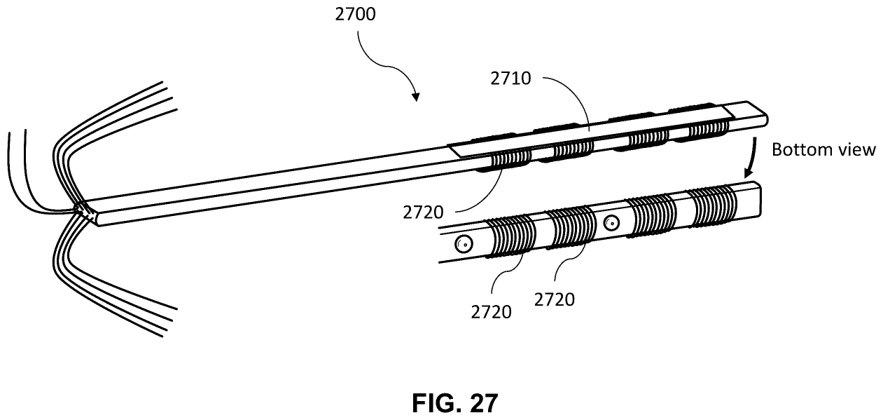

FIG. 27 illustrates an embodiment of a lead 2700 including coil electrodes 2720 that are wrapped around the lead. As shown, the electrodes can be coils wrapped around a portion of the distal portion of a lead that has two parallel planar surfaces. As used herein, the term “wrapped” means that the conductor (e.g., wire) is wound in a somewhat helical manner around the lead. The wrapping may have deviations from being a perfect helix in that the wrapping may be looser in some places and tighter others, for example, to facilitate flexible portions of the lead or to avoid obstruction or contact of other elements such as other electrodes. It is contemplated that while most implementations involve winding a conductor around the lead, it is also possible that equivalent structures can be used such as hollow bands, connected plates, etc. that can provide substantially the same circumferential coverage.

To provide directional stimulation capability consistent with the present disclosure, as shown in FIG. 27, there may be an insulating mask 2710 over a portion of the coils(s) on one of the parallel planar surfaces. Such a mask can be, for example, an electrically insulating or absorbing material (e.g., rubber, plastic, etc.) to prevent or reduce the transmittal of electrical energy. Such masking can be continuous as shown or can be segmented to only cover one or more individual electrodes. The masks need not be on the same side of the directional lead. For example, some electrodes may be masked on the top side, and other electrodes may be masked on the bottom side, thereby providing options for directional stimulation. Similarly, some implementations can have masking on multiple sides. For example, masking could be applied to three of the four sides of the depicted directional lead thus exposing the portion of the electrode on only one side.

While the embodiments of FIGS. 24-27 depict specific numbers and disposition of electrodes, it is contemplated that various other arrangements and dispositions may be utilized, for example, 1, 2, 3, 5, etc. electrodes arranged with varying spaces, etc.

Embodiments of leads disclosed herein (e.g., flat leads with directional electrodes) may, in some circumstances, experience minor twisting when deployed within a patient. The present disclosure thus contemplates a number of electrode configurations that assist in maintaining biological tissue contact and/or directionality of electrical energy from the electrodes towards the desired patient tissue.

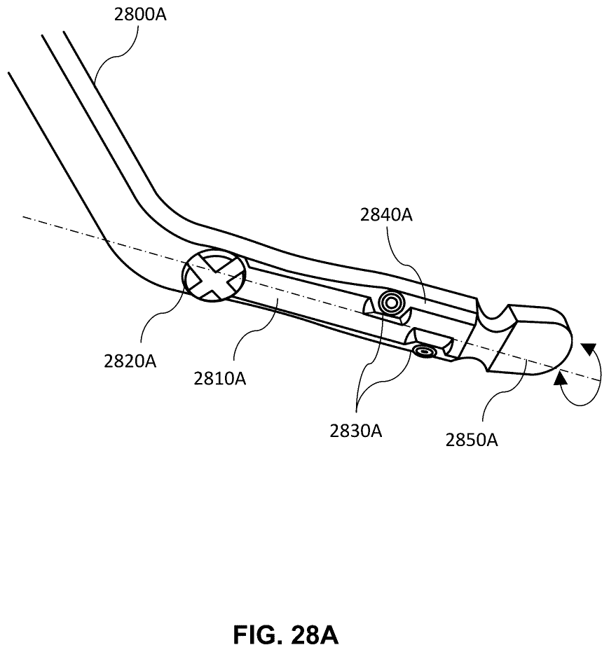

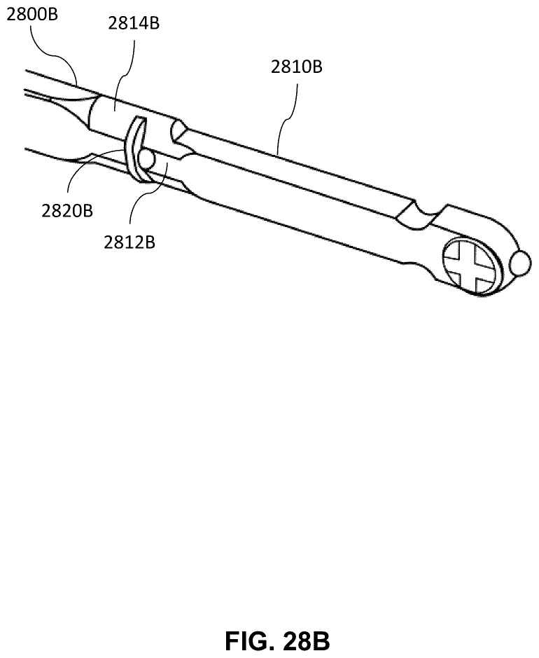

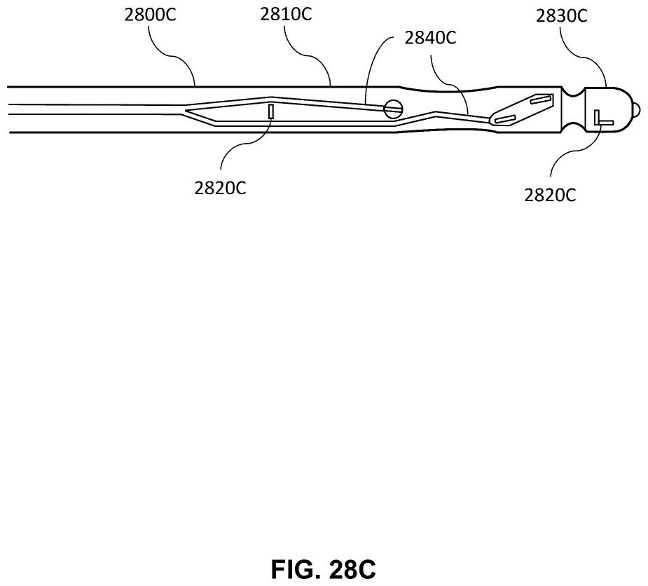

FIG. 28A illustrates an embodiment of a lead 2800A including electrodes 2830A that are angled and offset. Many lead embodiments herein include those where a distal portion has flat surface with electrodes on that surface intended to face the desired direction in the patient. The exemplary implementation of lead 2800A includes an electrode 2820A on flat surface 2810A and also electrode(s) 2830A that are oriented at angle(s) to the flat surface 2810A. The angles can vary (e.g., between 0 and ±90 degrees from perpendicular to the flat surface) but in some specific embodiments can include angles of (±) 5, 10, 30, 45, 60, or 75 degrees, with one example of electrodes being at (±) 45 degree angles, as depicted in FIG. 28A.

In some implementations, the lead can have material removed in places (such as along the edges of the flat surface) to create chamfers 2840A where the electrodes 2830A can be placed. Additionally, implementations may include protrusions that provide an angled surface for the electrodes 2830A to be disposed upon. Some implementations can include those where at least two electrodes are arranged at angle(s) to the flat surface and also offset from one another along the length of the distal portion, as shown in FIG. 28A.

These angled electrodes can act to keep the electrical energy directed properly if the lead is tilted or twisted (e.g., around a generally longitudinal direction as shown by the arrows around axis 2850A in FIG. 28A). Additionally, the features of two angled electrodes may be designed within a single electrode whereby two angled features of the single electrode keep the electrical energy directed properly if the lead is tilted or twisted.

FIG. 28B illustrates an embodiment of a lead 2800B including an electrode 2820B at least partially on the side of the lead. This exemplary lead 2800B can include a distal part 2810B that has a flat surface 2812B and a side surface 2814B. The electrode 2820B can be at least partially on the flat surface 2812B and extend at least partially over side surface 2814B.

In some embodiments, electrode 2820B could be rounded to at least partially extend over side surface 2814B. For example, the rounded electrode could follow the curve of a distal portion having a round cross section, or the electrode could include a rounded portion that wraps over an edge between a flat surface 2812B and side surface 2814B.

FIG. 28C illustrates an embodiment of a lead 2800C including radiopaque indicators 2820C which may be used to determine whether a lead is twisted or otherwise improperly oriented within a patient's body. In some embodiments, lead 2800C can include a distal portion 2810C that has one or more radiopaque indicators 2820C. Such radiopaque indicators can be distinctly visible to an imaging device (e.g., X-ray machines, fluoroscopes, MRIs, etc.) by having an increased opacity to an imaging modality relative to other areas of distal portion 2810C. In some implementations, lead 2800C can have at least two radiopaque indicators 2820C with one of the radiopaque indicators being at a distal end 2830C. In some implementations, as shown in FIG. 28C, the radiopaque indicator at distal end 2830C can form an L-shape. In further implementations, distal portion 2810C can include channels 2840C for holding cables for the electrodes. The channels can be at different depths or locations in the lead than the radiopaque indicators such that the cables do not interfere with the radiopaque indicators. For example, channel 2840C may go around radiopaque indicators 2820C such that the cables (which may have different opacity than the distal portion and/or the radiopaque indicator) is not in front of or behind the radiopaque indicator when viewed as in FIG. 28C.

FIGS. 29A-29Y below describe numerous embodiments for lead/electrode designs that facilitate pressing electrodes against patient tissue, fixing a lead within a patient, and spreading out electrodes upon deployment. The present disclosure contemplates that multiple of these lead design concepts can be combined in any particular lead embodiment. For example, disclosed features that facilitate spreading out of electrodes can be combined with leads that are configured to secure the lead and/or improve contact with biological tissue.

FIG. 29A illustrates an embodiment of a lead 2900A including a balloon 2920A for applying a downward force to a distal portion 2910A of the lead such that the lead will be pressed toward a patient's biological tissue (e.g., the portion of a pericardium 2970A intended to be stimulated by electrode 2950A).

As shown in FIG. 29A, distal portion 2910A can include a balloon 2920A on an upper face 2930A of the distal portion. The balloon 2920A can be configured to cause a downward force 2940A against the distal portion when the lead is deployed. The term “upper face” refers to a surface of the distal portion that is facing generally away from the intended direction of pressure on the lead. In the example of FIG. 29A, balloon 2920A can inflate to press against chest wall 2960A. The inflated balloon 2920A can then cause a downward force 2940A to push distal portion 2910A toward the intended contact surface (e.g., pericardium 2970A). Accordingly, the present disclosure distinguishes over balloons (or equivalent mechanisms) that are instead configured to specifically provide transverse forces on the lead or to separate tissue during lead advancement. The present disclosure also contemplates that other mechanisms and devices other than balloons can perform a similar function. For example, in some embodiments, the distal portion can include a spring on an upper face of the distal portion, with the spring configured to cause a downward force against the distal portion when the lead is deployed. The balloon 2920A can be designed to provide lead contact pressure while the lead is used. For example, the balloon can remain inflated for the entirety of the period of use of the lead. Alternatively, the balloon 2920A can be used for a only a portion of the period of lead implant time, in which the balloon 2920A can be used to establish lead position while allowing for tissue encapsulation of the lead, after which time the balloon 2920A can be deflated. Alternatively, the balloon 2920A can be used to expand framing structures 2980A that can provide long term force onto the lead to promote chronic contact pressure. An example of framing structures 2980A is shown in FIG. 29A. These framing structures 2980A can include metal or polymer materials that expand around the inflating balloon 2920A and remain expanded when the balloon is deflated.

FIG. 29B illustrates an embodiment of a lead 2900B including a wedge 2920B for applying a downward force to a distal portion 2910B of the lead. In such implementations, wedge 2920B can be configured to extend from the distal portion and cause a downward force against the distal portion when the lead is deployed, similar to the balloon embodiment described with respect to FIG. 29A. As shown by the inset 2901B (depicting an end view of the lead), wedge 2920B can extend upward from the distal portion (e.g., towards chest wall 2960B). In some implementations, wedge 2920B can also extend laterally from distal portion 2910B, as shown. In some implementations, wedge 2920B can extend longitudinally along distal portion (in addition to extending upward). Wedge 2920B can be connected anywhere on the distal portion, such as the sides (as shown), an upper face, etc. The present disclosure contemplates that any number of wedges can be included along the lead to provide the downward force, for example, two wedges as depicted in FIG. 29B, but also possibly only one, or three, or more.

The present disclosure contemplates other implementations that provide pressure between portions of the lead and the desired patient anatomy (e.g., the pericardium). For example, the distal portion of the lead can include a helical coil portion configured to be compressed prior to implantation and released when the lead is deployed, similar to a helical spring. In this manner, release of the coil portion within the body can cause the distal portion of the lead to be forced against patient anatomy (e.g., the pericardium). The coil portion can have a predetermined coil shape that can be held in a stretched or compressed shape for implantation.

Leads described herein may be loaded into or onto a delivery system and then deployed into a patient. As used herein, the term “deployed configuration” refers to a configuration intended to be taken by a lead when lead is deployed into a patient and no longer confined to a delivery system or restrained by a stylet. The deployed configuration may also be achieved when a distal portion of the lead includes a shape memory material that has been activated (e.g., by heat). Deployment can take place, for example, when a delivery system is activated by a user to advance a lead into a patient. In contrast, the term “loaded configuration” refers to the lead configuration taken when a lead is loaded inside a delivery system, restrained by a stylet, etc.

FIG. 29C illustrates an embodiment of a lead 2900C having an elastically deformable portion configured to have one point of contact for pushing against a chest wall 2960C in order to facilitate contact between electrode 2950C and patient tissue. The exemplary lead 2900C can have a distal portion 2910C that includes a fixed portion 2912C configured to be affixed to a patient in order to retain fixed portion 2912C in place. For example, fixed portion 2912C can include suture holes 2922C or grooves for suturing the fixed portion to the patient. Distal portion 2910C can also include an elastically deformable portion 2952C configured to maintain contact between electrode 2950C and biological tissues (e.g., pericardium 2970C) during heart movement when in a deployed configuration. As shown in FIG. 29C, lead 2900C can be shaped to have a first point of contact 2980C with the patient at chest wall 2960C. During tissue movement (for example, pericardium 2970C moving as the heart beats), the restoring force caused by the lever shape of the lead pushes down and the elastically deformable portion 2952C facilitates maintaining contact between electrode 2950C and pericardium 2970C while providing sufficient flexibility to avoid damaging the pericardium 2970C.

FIG. 29D illustrates an embodiment of a lead 2900D configured to have two points of contact for pushing against a chest wall 2960D. In this implementation, the lead can be shaped to, when the lead is deployed, have a first point of contact 2980D with the patient at a chest wall and a second point of contact 2990D with the chest wall 2960D. The first point of contact can be at a distal end 2930D of the distal portion 2910D and the second point of contact 2990D can be at a proximal point 2932D of distal portion 2910D, with the electrode 2950D disposed between proximal point 2932D and the distal end 2930D. For example, second point of contact 2990D can be formed by including one or more additional bends in the lead 2900D. The operation and benefits of lead 2900D are otherwise similar in many respects to those of lead 2900C.

FIGS. 29E and 29F illustrate lead-design embodiments that facilitate engagement between a lead and biological tissue (with the example of FIG. 29E utilizing a suction cup design and FIG. 29F utilizing tines). In general, some such implementations can have a distal portion of the lead include a connecting portion that has a contacting edge extending from the distal portion of the lead (so as to extend towards the biological tissue desired for contact) and be configured to, in operation, pull the distal portion towards the biological tissue by engagement of the contacting edge with the biological tissue. By “pull,” the present disclosure means to create a force tending to bring the distal portion of the lead and the biological tissue together. As used herein, the “connecting portion” includes any mechanism that can act to connect or secure a portion of the lead to the biological tissue where electrode contact is desired. Furthermore, any such connecting portion includes a “contacting edge” which, as used herein, broadly covers a structural feature that directly engages the biological tissue. However, the term “contacting edge” should not be strictly interpreted to be an “edge” in a colloquial sense (e.g., a sharp or narrow feature). For example, as described below, the one example of a connecting portion refers to a suction cup with a contacting edge being the rim of the suction cup.

The embodiment of FIG. 29E illustrates a lead 2900E including a suction cup 2920E for pulling the lead against biological tissue. FIG. 29E illustrates that the connecting portion can be a suction cup 2920E opening towards the biological tissue that, when abut against patient tissue below distal portion 2910E of the lead and a reduced pressure (e.g., a vacuum) is formed in the suction cup, there will be downward pressure on the distal portion 2910E. To create the vacuum, in some implementations, the suction cup 2920E can be pressed against biological tissue and the vacuum formed by mechanically pushing out air and reducing the volume inside the suction cup 2920E. In other implementations, the lead can include a gas duct extending through the lead to the suction cup 2920E. The gas duct can be operatively connected to a vacuum pump that can evacuate air in the suction cup through the gas duct and thereby create the suction between the lead and biological tissue.

FIG. 29F illustrates an embodiment of a lead 2900F that includes tines 2920F for pulling the lead against biological tissue. In this embodiment, connecting portion can include tines 2920F that are configured to engage the biological tissue and hold the distal portion 2910F and the electrode 2950F against the biological tissue. As used herein, a “tine” is a device for grabbing onto tissue, and can include hooks, barbs, or the like. The tines can grab the tissue without puncturing it, or in some embodiments may puncture the tissue in a benign manner to secure the lead in place. In some implementations, lead 2900F can include a stylet cavity 2921F formed within the distal portion of lead 2900F and shaped to receive a stylet 2910F to facilitate delivery and engagement of lead 2900F. The connecting portion can be configured to be held in an open configuration by the stylet 2910F and, when the stylet is removed, tines 2920F can extend through one or more apertures 2922F in the distal portion. In some implementations, tines 2920F can be electrically controlled to close upon (e.g., grab) the biological tissue. In some implementations, tines can also be electrically conductive (e.g., metallic) to facilitate the delivery of electrical energy from electrode 2952F, through the tines 2920F, to the biological tissue 2970F with which they engage.

In some implementations, a distal portion of lead may be made from a soft/pliable material such as silicone, rubber, flexible plastic, etc., in order to more closely follow the shape of the biological tissue to which it may be fixed. Accordingly, any of the disclosed lead concepts (e.g., those facilitating fixation to the pericardium) can include a distal portion of the lead being made from a soft/pliable material.

The present disclosure also contemplates numerous embodiments for leads that spread out electrodes. When endeavoring to have electrodes contact biological tissues, it can be helpful to expand the geometric spread of the potential electrodes to be used for stimulation. For example, multiple electrodes can be spaced along the distal portion of the lead, the proximal portion of the lead, or both.

Lead embodiments that include multiple electrodes can further implement designs where sets of electrodes are optimally selected in order to best deliver therapeutic energy. For example, a lead can be configured to deliver electrical energy with one or more sets of electrodes, such as different electrodes spread out on the lead. The system may further include a connector having multiple poles corresponding to the multiple electrodes. The connector can then be configured to provide the therapeutic energy from a pulse generator to certain sets of the multiple electrodes. In some implementations, the lead can include a manual switch that configures the connector to deliver the therapeutic energy through a selected set of the multiple electrodes. In still further implementations, the pulse generator or other device having a computer processor can automatically select sets of electrodes to be energized with the pulse generator.

In some embodiments, electrodes can be configured as bands or rings that can fully or substantially (e.g., greater than 180°) encircle the lead. Such electrodes can therefore be configured to provide therapeutic energy over a wide angle which may reach the desired tissue even if the lead may be tilted relative to the target location. However, in other embodiments, to provide improved directionality of therapeutic energy, a lead can include an electrically insulating portion around at least part of a circumference of the lead, the electrically insulating portion configured to insulate surrounding muscle and/or tissue from the therapeutic energy. In this way, the therapeutic energy can be directed over a smaller angle due to the insulation blocking the therapeutic energy from being delivered in other directions.

FIG. 29G illustrates an embodiment of a lead 2900G having a coiled shape. As shown in the embodiment of FIG. 29G, the distal portion 2910G can have a coil shape that spreads out the multiple electrodes 2950G when lead 2900G is in a deployed configuration. As used herein, when stating that electrodes are “spread out,” this means that, in the deployed configuration, the electrodes will be spread over some length and width, the length and width being dimensions across the surface of the biological tissue (e.g., pericardium) to which the therapeutic energy is to be delivered. In the example of FIG. 29G, if the coil shape of the distal portion 2910G was placed against the pericardium, the electrodes 2950G would be spread out on the surface of the pericardium in both a length dimension and width dimension. The electrodes may exhibit some spread over a height dimension as well. Such coil-shaped leads need not be specifically circular or coiled about a straight line and the present disclosure contemplates that the coil shape can be less than, equal to, or greater than 360°. Electrodes 2950G can be distributed along the length of the distal portion 2910G and thereby may be spread out in a variety of configurations based on the electrode locations and lead coiling geometry.

FIG. 29H illustrates an embodiment of a lead 2900H having a spiral shape. As shown in the embodiment of FIG. 29H, the distal portion 2910H can have a spiral shape that spreads out the multiple electrodes 2950H when lead 2900H is in a deployed configuration. Inset 2924H also depicts a simplified cross section of lead 2900H through one electrode 2950H. In this example, electrodes 2950H may only cover part of the circumference of the lead and thereby can be somewhat directional in their ability to provide therapeutic energy to nearby tissue. In this way, electrodes 2950H may be configured on distal portion 2910H pointed somewhat inward and downward to direct the therapeutic energy toward a target location generally coincident with center 2926H of the spiral and below it (e.g., where biological tissue may be if the lead were placed, e.g., against the pericardium). In addition to electrodes distributed along distal portion 2910H, in some embodiments, there can also be an electrode at the center 2926H of the spiral and directed downward.

FIG. 29I illustrates an embodiment of a lead 2900I having a wavy shape. As shown in the embodiment of FIG. 29I, the distal portion 2910I can have a wavy shape that spreads out multiple electrodes 2950I when the lead is in a deployed configuration. As used herein, the term “wavy shape” is a broad characterization of the lead shape that is not a straight line but also not approximating a particular geometric shape. However, a “wavy shape” can still act to spread out electrodes and thereby can provide benefits similar to other implementations herein that also spread the electrodes out.

Any of the lead designs discussed herein can be configured such that they assume a predetermined shape when deployed by a delivery system. In some cases, the direction can be determined or facilitated by the design of the delivery system (e.g., implementations discussed herein where leads are directed utilizing ramps). In other implementations, the direction or shape may be determined by the design of the lead itself (e.g., a lead with a preformed shape that is forced to be held straight when within delivery system 200 but that assumes the preformed shape again upon exiting the delivery system). The present disclosure also contemplates leads being delivered over a stylet which can similarly hold a lead with a preformed shape until the stylet is removed and the lead reverts back to its preformed shape. Accordingly, in some embodiments, lead 2900I can be flexible and include a stylet cavity 2924I shaped to receive a stylet to facilitate delivery of lead 2900I. In some implementations, distal portion 2910I can further include one or more barbs 2926I extending from the distal portion and shaped to engage biological tissue.

FIG. 29J illustrates an embodiment of a lead 2900J having an electrode extension 2920J. The present disclosure contemplates lead designs that include multiple electrodes and where the distal portion 2910J of lead 2900J includes an electrode extension 2920J having a tip electrode 2922J. The electrode extension 2920J can be configured to increase a distance between the tip electrode 2922J and another electrode 2950J on the distal portion of the lead and/or to facilitate contact of the tip electrode 2922J with biological tissue (e.g., pericardium 2970J) of the patient when the lead is in a deployed configuration. Such designs can be included with any of the embodiments disclosed herein, for example, the single paddle or “L-shaped” lead such as depicted in FIG. 29J, splitting leads, planar leads, etc. As shown in FIG. 29J, electrode extension 2920J may be flexible, which can assist with encouraging contact with, but not perforation of, biological tissue 2970J. However, it is also contemplated that the electrode extension can be rigid or have an intermediate flexibility to assume a preformed shape when not constrained by a delivery system, biological tissue, etc.

Many of the electrode extensions disclosed herein can be generally described as a short extension out of the main lead body. Electrode extensions are thus distinguishable from the splitting lead designs discussed below, which generally include more significant structures (e.g., for supporting multiple electrodes) and where the splitting lead is implanted with assistance from additional delivery system features (e.g., ramps that separate the splitting lead sub-portions). Numerous embodiments herein (e.g., with reference to FIG. 29K-S) can include features such as tip electrodes on the electrode extensions.

In some embodiments, the lead (e.g., 2900J) can also include a cavity 2924J in proximal part 2912J and/or a distal part 2914J of distal portion 2910J, which can be shaped to receive the electrode extension 2920J when lead 2900J is configured in a loaded configuration. Such a loaded configuration can occur when, for example, a lead is loaded into a delivery system or held in place by a stylet during delivery through an opening in the patient. Then, at some point during lead deployment, electrode extension 2920J can emerge from lead 2900J. For example, electrode extension 2920J can emerge from the cavity in distal part 2914J as it bends upward to form more of an “L-shape.”

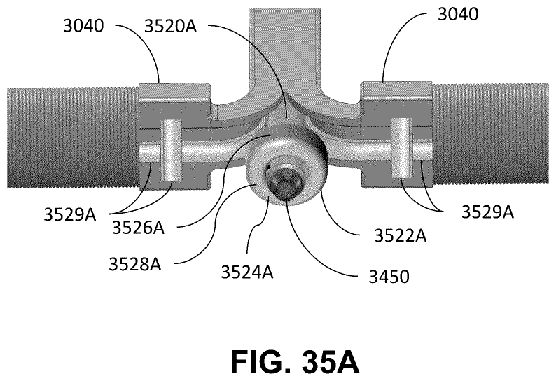

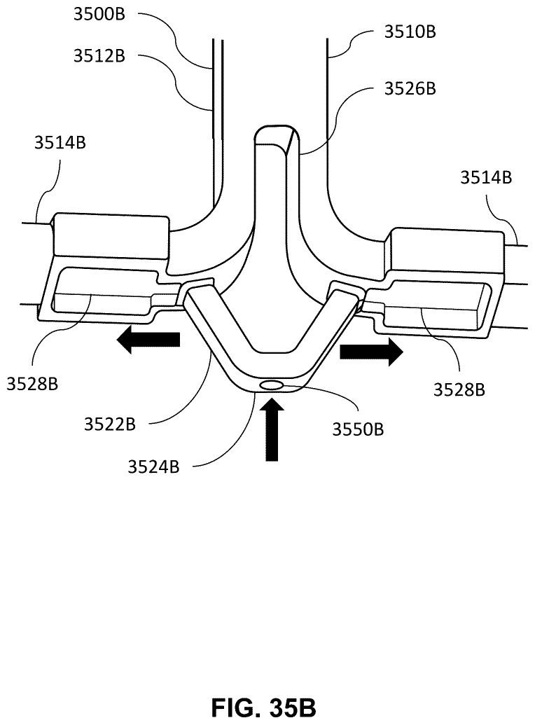

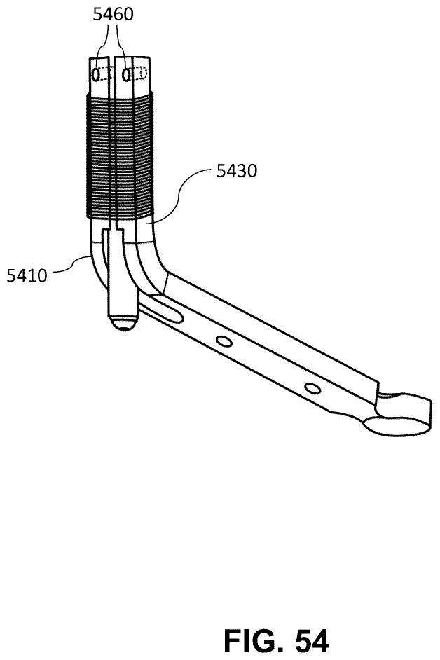

In the side views of the leads shown in many of the following embodiments, simplified depictions of the cavities are shown with dashed lines. In addition to FIG. 29J, FIG. 35A and FIG. 54 show perspective views exemplary cavities. In various embodiments, cavity 2924J can extend through the lead completely to form an aperture, or only partially to form more of a recess. As described in detail below, the present disclosure contemplates numerous embodiments of electrode extensions, cavities, and the other parts of the lead associated with them. While these exemplary embodiments are described for a lead having a single distal part, similar features can be implemented in any of the leads disclosed herein (e.g., splitting leads).

FIG. 29K illustrates an embodiment of a lead 2900K having an electrode extension 2920K. In the embodiment of FIG. 29K, electrode extension 2920K can be coupled to distal part 2914K of distal portion 2910K and, in the deployed configuration, extend at an angle 2928K away from distal part 2914K. An example of cavity 2924K formed in lead 2900K and shaped to receive electrode extension 2920K is also shown in FIG. 29K.

FIG. 29L illustrates an embodiment of a lead 2900L having an electrode extension 2920L. In the embodiment of FIG. 29L, electrode extension 2920L can be coupled to proximal part 2912L of distal portion 2910L and, in the deployed configuration, extend at an angle 2928L away from proximal part 2912L. An example of cavity 2924L formed in lead 2900L and shaped to receive electrode extension 2920L is also shown in FIG. 29L.

FIG. 29M illustrates an embodiment of a lead 2900M having an electrode extension 2920M. In the embodiment of FIG. 29M, electrode extension 2920M can be coupled to proximal part 2912M of distal portion 2910M and, in the deployed configuration, extend at an angle 2928M away from proximal part 2912M. Electrode extension 2920M can further include an elbow 2926M, the elbow acting to direct electrode 2950M in more of a downward direction. An example of cavity 2924M formed in lead 2900M and shaped to receive electrode extension 2920M is also shown in FIG. 29M.

FIG. 29N illustrates an embodiment of a lead 2900N having an electrode extension 2920N. In the embodiment of FIG. 29N, electrode extension 2920N can be coupled to proximal part 2912N of distal portion 2910N and, in the deployed configuration, have a horizontal extension 2926N and a vertical extension 2928N. An example of cavity 2924N formed in lead 2900N and shaped to receive electrode extension 2920N is also shown in FIG. 29N.

FIG. 29O illustrates an embodiment of a lead 2900O having an electrode extension 2920O. In the embodiment of FIG. 29O, electrode extension 2920O can be coupled to proximal part 2912O of distal portion 2910O and, in the deployed configuration, having a C-shape and a vertical extension 2928O. An example of cavity 2924O formed in lead 2900O and shaped to receive electrode extension 2920O is also shown in FIG. 29O.

FIG. 29P illustrates an embodiment of a lead 2900P having an electrode extension 2920P. In the embodiment of FIG. 29P, electrode extension 2920P can be coupled to proximal part 2912P of distal portion 2910P, and in the deployed configuration the electrode extension ending flush with distal part 2914P of distal portion 2910P with only the tip electrode 2922P protruding beyond distal part 2914P. An example of cavity 2924P formed in lead 2900P and shaped to receive electrode extension 2920P is also shown in FIG. 29P.

FIG. 29Q illustrates an embodiment of a lead 2900Q having an electrode extension 2920Q. In the embodiment of FIG. 29Q, electrode extension 2920Q can be coupled to distal part 2914Q of distal portion 2910Q and, in the deployed configuration, extending substantially coplanar to distal part 2914Q. An example of cavity 2924Q formed in lead 2900Q and shaped to receive electrode extension 2920Q is also shown in FIG. 29Q. In this embodiment the term “substantially coplanar” can include the electrode extension being at an angle 2928Q of ±30 degrees from distal part 2914Q, as shown in FIG. 29Q. In particular embodiments, angle 2928Q can be 5, 10, 15, 20, 25, or 30 degrees, such that electrode 2922Q can be directed toward a target such as the pericardium.

FIG. 29R illustrates an embodiment of a lead 2900R having an electrode extension 2920R. In the embodiment of FIG. 29R, electrode extension 2920R can be coupled to and aligned with distal part 2914R of distal portion 2910R. An example of cavity 2924R formed in lead 2900R and shaped to receive electrode extension 2920R is also shown in FIG. 29R. As such, when lead 2900R is in a loaded configuration, the electrode extension 2920R can be within the cavity. When deploying into a deployed configuration, electrode extension 2920R can deploy while remaining aligned with the deploying distal part 2914R.

FIG. 29S illustrates an embodiment of a lead 2900S having an electrode extension 2920S. In the embodiment of FIG. 29S, front view 2902S shows that electrode extension 2920S can be wider than a width 2926S of tip electrode 2922S. This embodiment contrasts with some other embodiments that depict the electrode as being approximately the same width as the electrode extension. As shown in side view 2901S, electrode extension 2920S can be coupled to proximal part 2912S of distal portion 2910S. An example of cavity 2924S formed in lead 2900S and shaped to receive electrode extension 2920S is also shown by side view 2904S. In the example shown, cavity 2924S is different than depicted in previous embodiments by it extending across the entire width of distal part 2914S. Such a cavity allows a greater width of electrode extension 2020S without electrode extension 2020S extending (laterally) beyond distal part 2914S. As with any of the embodiments of the electrode extensions described herein, electrode extension 2920S can be rigid or flexible.

FIG. 29T and FIG. 29U illustrate embodiments of a lead having multiple sub-portions that facilitate lead stabilization that could be referred to as 2-prong or 3-prong “chicken foot” designs. Such leads can be different from the splitting lead designs discussed below, which generally include more significant sub-structures and where the lead is implanted with assistance from additional delivery system features (e.g., ramps that separate the splitting lead's sub-portions). For example, embodiments of the distal parts shown in FIG. 29T and FIG. 29U can be more similar to the electrode extensions described herein and may emerge from a single port on the delivery system. Such leads can also be semi-rigid, meaning they are not as flexible as a wire or cable lead, but also not entirely rigid, as with some embodiments described herein that may use ramps to deflect more rigid sub-portions in different directions. The semi-rigid lead portions can flex during loading, deployment, or in response to tissue motion (e.g., motion of the pericardium), but generally have a preferred shape such that there is some resistance to deformation.

FIG. 29T illustrates an embodiment of a lead 2900T having two sub-portions 2920T that facilitate lead stabilization. In the embodiment of FIG. 29T, distal portion 2910T can configured as two sub-portions 2920T that extend in different directions when in a deployed configuration. Sub-portions 2920T can be semi-rigid. In some embodiments, each of the two sub-portions 2920T can include an anode and a cathode. The two sub-portions 2920T can have an angle 2924T of at most 60 degrees from a center axis 2922T in one implementation. Other embodiments can have angle 2924T being 10, 20, 30, 75 or 90 degrees.

FIG. 29U illustrates an embodiment of a lead 2900U having three sub-portions that facilitate lead stabilization. In the embodiment of FIG. 29U, distal portion 2910U can be configured to split apart into three sub-portions that extend in different directions when in a deployed configuration. The sub-portions can be semi-rigid. In some embodiments, the three sub-portions can have an angle of at least 180 degrees between two sub-portions on either side of a third sub-portion. In one embodiment, first sub-portion 2922U and second sub-portion 2924U can each include a cathode and a third sub-portion 2926U can include an anode.

FIG. 29V illustrates an embodiment of a lead 2900V having electrodes on separate sub-portions. In the embodiment of FIG. 29V, lead 2900V is similar to that in 2900U but with multiple sub-portions 2920V including a cathode 2950V, and the lead 2900V including an anode 2952V proximate a central region 2922V of lead 2900V where sub-portions 2920V meet. In various implementations, anode 2952V can be on a sub-portion or on an electrode extension. In some embodiments, the above configuration can be reversed, where multiple sub-portions can include an anode, and the lead can include a cathode proximate a central region of the lead where the sub-portions meet. In various implementations, a cathode can be on a sub-portion or on an electrode extension.

FIG. 29W illustrates an embodiment of a lead 2900W having an electrode 2950W on one sub-portion and laterally-extending portions 2923W/2924W on that same sub-portion in order to facilitate stabilization when deployed. In the embodiment of FIG. 29W, lead 2900W is similar to that of 2900V with distal portion configured to split apart into sub-portions 2920W that travel in multiple directions during implantation into the patient. Such embodiments can also have at least one of the sub-portions including a laterally-extending portion 2922W that can facilitate stabilization of the lead sub-portion within a patient's body. The example of FIG. 29W depicts an example where there are two laterally-extending portions 2923W/2924W extending in either direction laterally from the sub-portion 2920W. While the example of FIG. 29W shows laterally-extending portions with reference to a lead having multiple sub-portions, the present disclosure contemplates the use of such laterally-extending portions with any of the disclosed leads herein (e.g., on a distal portion of lead 2000 in FIG. 20A).

In some embodiments, lead 2900W can include a cathode 2950W and an anode 2952W. As also shown in FIG. 29W, some lead embodiments including multiple sub-portions may have at least one sub-portion 2920W not including any electrodes. Such sub-portions can be included primarily for stabilizing the lead against twisting or tilting.

FIG. 29X illustrates an embodiment of a lead 2900X having two independently elastically deformable sub-portions 2920X/2922X configured to facilitate contact between electrodes 2950X and 2952X with patient tissue. The lead 2900X may include fixation features that allow the lead to be fixed into patient tissue. In some implementations, fixation may occur in a manner that results in an initial preloading (i.e., elastic deformation) of the sub-portions. The lead can be implanted such that the elastically deformable segments can be flexed near the middle of their flexible range, which can aid in maintaining tissue contact as the tissue moves closer to, and further away from, the lead. The exemplary lead 2900X can have a longer sub-portion 2920X that can include electrode 2950X as well as a shorter sub-portion 2922X that can include a separate electrode 2952X. Each sub-portion 2920X and 2922X can independently elastically deform in order to maintain tissue contact as the heart moves relative to the fixed lead position during cardiac contraction or during postural movements of the patient. The two sub-portions 2920X/2922X may be of dissimilar lengths (e.g., as shown) in order to improve the contact location of the electrode relative to the heart. In some embodiments, the surface area of electrode 2950X of the longer sub-portion 2920X may be smaller than the surface area of electrode 2952X of the shorter sub-portion 2922X. Further embodiments may switch the locations of the dissimilar surface area electrodes (i.e., put electrode 2952X on sub-portion 2920X and electrode 2950X on sub-portion 2920X) or include sub-portions of lead 2900X being matched lengths.

Furthermore, either electrode 2950X or 2952X may include features that promote tissue contact as the lead tilts or twists, including features that angle the electrode from a flat surface 2912X of the lead sub-portion or wrap portions of the electrode (e.g., electrode 2952X) to a side surface 2914X of the sub-portion. Also, instead of one electrode, as shown, some embodiments can have two or more electrodes on one or both sub-portions to allow for additional angles from flat surfaces to further promote tissue contact with tilting and/or twisting motions.

FIG. 29Y illustrates an embodiment of a lead 2900Y having a “heel portion” 2920Y. In FIG. 29Y, the distal portion 2910Y of lead 2900Y is configured to include heel portion 2920Y that can facilitate contact of an electrode 2922Y (located on the heel portion 2920Y) with the biological tissue of a patient (when the lead 2900Y is in a deployed configuration). The heel portion 2920Y can be disposed generally near the intersection of the proximal part 2912Y (of the lead's distal portion 2910Y) and the distal part 2914Y (of the lead's distal portion 2910Y). As shown in the example of FIG. 29Y, heel portion 2920Y can, in some embodiments, be considered as included directly in the distal part 2914Y. While the dashed boxes of FIG. 29Y generally indicate the distal portion 2910Y, proximal part 2912Y, distal part 2914Y, and heel portion 2920Y, these boxes are for illustrative purposes only and should not be considered to rigidly describe or limit the bounds or extent of any particular element.

In some embodiments, heel portion 2920Y can be formed by a bend 2924Y in the distal part 2914Y of the lead 2900Y that facilitates contact of the electrode 2922Y located on the heel portion 2920Y with the biological tissue (e.g., the pericardium) of a patient when the lead 2900Y is in the deployed configuration. As shown in FIG. 29Y, bend 2924Y extends electrode 2922Y further in the vertical direction (e.g., towards the tissue to be contacted) than the remainder of distal part 2914Y. This vertical extension, which may vary with the embodiment (e.g., 0.1 cm., 0.5 cm., 1.0 cm., etc.), can provide additional pressure between electrode 2922Y and the biological tissue. Bend 2924Y can be a curved section as shown, but may also be formed as a “V” shape, a “U” shape, etc.

FIG. 29Y-1 illustrates an embodiment of a lead 2900Y-1 having a bend 2926Y in proximal part 2912Y (note: where possible, element numbers are repeated for similar components of those shown in FIG. 29Y). As shown in this exemplary embodiment, proximal part 2912Y includes a bend 2926Y to place a vertical portion 2928Y of the proximal part 2912Y closer to a distal tip 2952Y of the lead 2900Y when the lead is in a deployed configuration. Such bends can configure the lead to direct a downward force from the proximal part 2912Y to a location directly over an electrode or further distal along the lead to improve the contact pressure to the biological tissue and facilitate contact of the electrode with biological tissue when the lead is in the deployed configuration. Additionally, a configuration can, for example, prevent the distal part from sliding along the surface of biological tissue and/or can reduce lifting of electrode 2950Y upwards away from the biological tissue.

As shown in FIG. 29Y-1, electrical lead 2900Y-1 can have a bend that places the vertical portion 2928Y approximately over electrode 2922Y on the distal part 2914Y. This is depicted by the center of the vertical portion 2928Y of proximal part 2912Y being along line 2954Y that is approximately vertical and in line with electrode 2922Y. FIG. 29Y-1 shows both a bend 2926Y in the proximal part 2912Y and also a bend 2924Y to form a heel portion 2920Y (as discussed with regard to FIG. 29Y). It is contemplated that lead designs herein may include none, either one, or both of these bends. For example, some embodiments may have bend 2926Y (to locate the proximal part to be more towards the distal tip) but not have bend 2924Y (such that distal part 2914Y is largely flat except near electrode 2950Y).

FIG. 29Y-2 illustrates an embodiment of a lead 2900Y-2 having a vertical portion 2928Y of proximal part 2912Y of the lead configured to be placed more distally than an electrode 2922Y in the deployed configuration. In such embodiments, bend(s) 2926Y can configure lead 2900Y-2 to place vertical portion 2928Y closer to the distal tip 2952Y than an electrode 2922Y on the distal part 2914Y, for example with an offset 2956Y. The offset 2956Y in the distal direction from line 2954Y going through electrode 2922Y can vary across different lead designs, for example being 0.1 cm., 1.0 cm., 2.0 cm., etc. In some embodiments, such as shown in FIG. 29Y-2, the proximal part 2912Y can have additional or larger bends, for example, resulting in the proximal part 2912Y including an S-shaped section.

In some embodiments, the bend(s) 2926Y can be configured to increase the flexibility of proximal part 2912Y to facilitate maintaining contact with biological tissue when the lead is in the deployed configuration. For example, in addition to increasing flexibility by virtue of the inclusion of non-vertical sections, bend(s) 2926Y may also be constructed of a more flexible material, be smaller in cross section, have cavities, cutouts, etc., to increase flexibility. As such, in embodiments where a lead is implanted with some compression of the flexible portion, the bend(s) may expand to maintain contact between electrode 2922Y and biological tissue, for example, in response to a heart contraction or other movement.

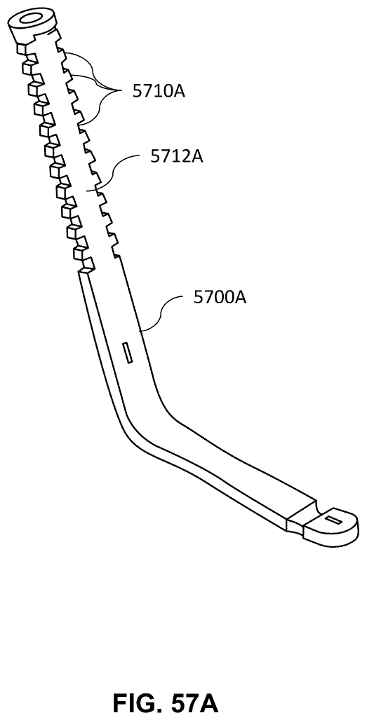

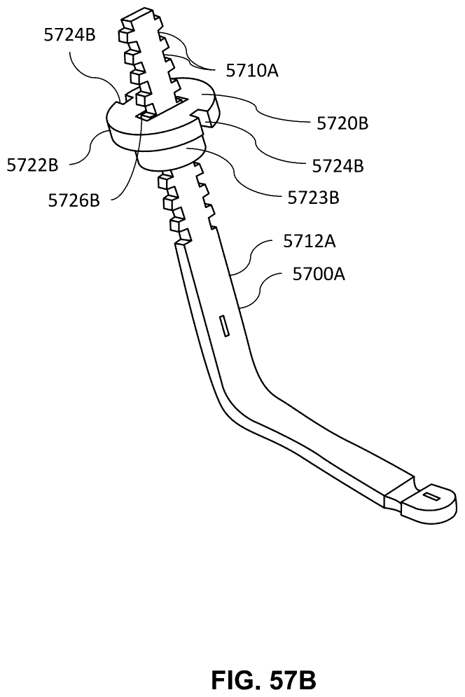

In some embodiments, proximal part 2912Y can include one or more grooves or holes for suturing the vertical portion 2928Y to the patient. Examples of such grooves or holes that can be incorporated can be seen in FIGS. 54 and 57. The grooves or holes, with the above-described flexibility incorporated through the bend(s), allow the lead to remain securely implanted, yet expand/contract to maintain electrode contact with the biological tissue, with bends 2926Y allowing the contact force to be applied in a manner directly over electrode 2922Y, or more distal or proximal depending upon the shape of bends 2926Y.

In addition to the lead designs previously presented, the present disclosure also contemplates splitting leads whereby a delivery tool can facilitate different portions of the splitting lead spreading out in a particular manner during delivery (e.g., separating sub-portions similar to those described above).

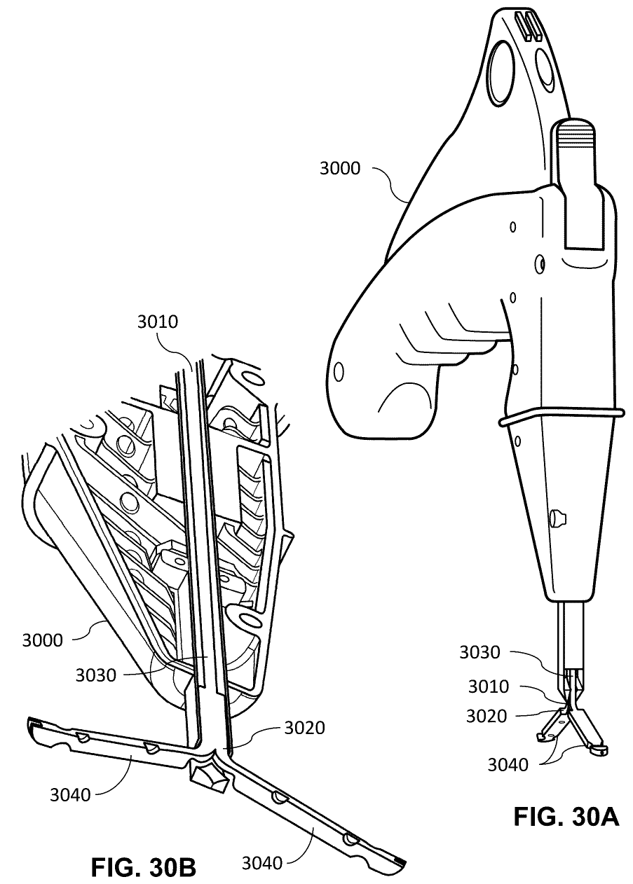

In another lead embodiment, depicted in FIGS. 30A and 30B, an electrical lead 3010 may be configured to have its distal portion split apart into two or more significant portions and travel in different directions during implantation in a patient (e.g., as a result of engaging with ramps, as described further below). Such designs are referred to herein as “splitting leads.” FIGS. 30A and 30B depicts one exemplary embodiment of a splitting lead.

Similar to other leads of the present disclosure, the splitting lead can have a distal portion 3020 having electrode(s) that are configured to generate therapeutic energy for biological tissue of the patient. The electrodes can include any combination of defibrillation electrodes and/or cardiac pacing electrodes. Also, as partially shown in FIG. 30B, the lead can have a proximal portion 3030 coupled to the distal portion and configured to engage a controller. The controller can be configured to cause the electrode(s) to generate the therapeutic energy, e.g., via transmitting current through wires to the various electrodes similar to other disclosed embodiments such as that of FIG. 24.

In the depicted embodiment, the distal portion is configured to split apart into sub-portions 3040 that travel in multiple directions during implantation into the patient. In this example, a delivery system 3000 is inserted into a patient (e.g., through an intercostal space in the region of the cardiac notch) and, after insertion, lead 3010 is advanced and sub-portions 3040 of the lead split off in different directions. While the example of FIGS. 30A and 30B depicts the lead splitting off in two different directions, the present disclosure contemplates designs following the teachings herein that split off in more directions (e.g., three directions, four directions, etc.).

The splitting lead designs disclosed herein may be particularly useful for ICD/defibrillation applications as they can provide for additional lead length and thus additional area for electrode surface. However, the present disclosure contemplates the use of splitting lead designs in pacing applications as well. In some applications, the splitting lead designs disclosed herein can include both pacing and defibrillation electrodes, as taught throughout this disclosure.

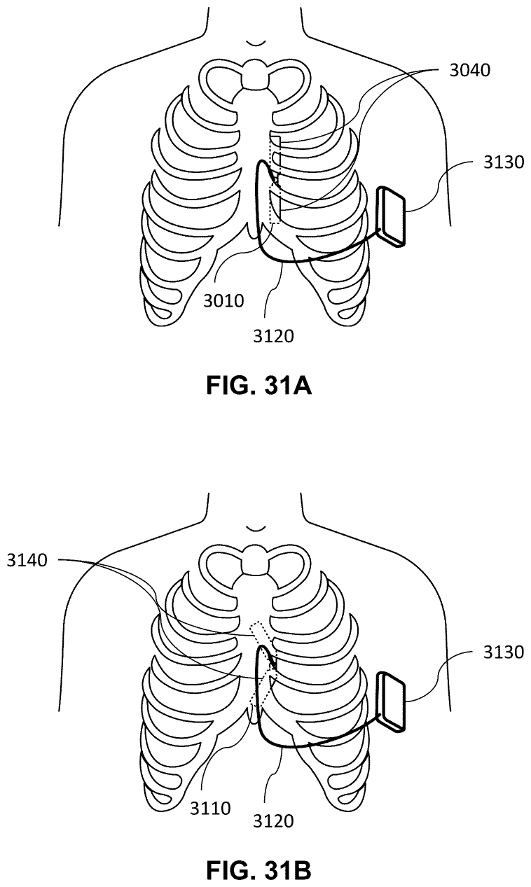

FIG. 31A depicts an exemplary placement for a splitting lead 3010 in which a lead delivery system (or merely “delivery system”) can be inserted into the patient, for example, through an intercostal space associated with or in the region of the cardiac notch of the patient. Exemplary methods of placing the splitting lead can include operating the delivery system to place the distal portion of the lead in an extravascular location of the patient. For example, the extravascular location can be in a mediastinum of the patient, in the region of the cardiac notch, and/or on or near the inner surface of an intercostal muscle. The lead's wires 3120 can extend to a controller 3130, which may be implanted in the patient.

After insertion, the delivery system 3000 can be operated such that lead 3010 can be advanced so that the distal portion of lead 3010 splits apart into two portions that travel in multiple directions within the patient. As shown in FIG. 31A, the distal portion of lead 3010 can split so that sub-portions 3040 travel in opposite directions parallel to a sternum of the patient.

FIG. 31B depicts another exemplary placement for a splitting lead 3110 where the distal portion of the lead splits apart into two sub-portions 3140 that travel in directions approximately 100° apart and under the sternum of the patient. Additional extravascular placements are contemplated and can include the distal portion of the lead splitting into more sub-portions (e.g., the distal portion of the lead may split into three portions that travel in directions approximately 90° apart and parallel or perpendicular to the sternum of the patient.

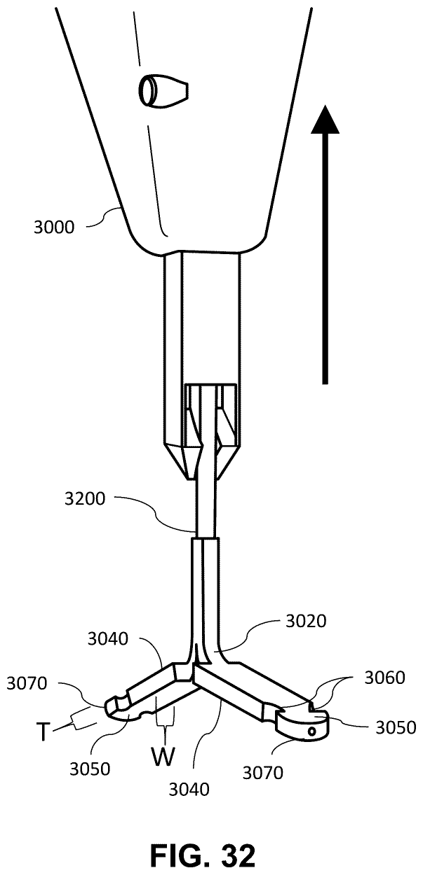

FIG. 32 illustrates another view of an exemplary splitting lead, exiting an exemplary delivery system 3000. Such splitting leads can allow for increased total length and electrode surface area while facilitating implantation.

In one embodiment, the distal portion of the lead can be configured to split apart into two sub-portions having a combined length of approximately 6 cm (e.g., ± up to 1 cm). Numerous other lengths are contemplated, for example, approximately 4, 5, 7, 10, etc., centimeters. The two sub-portions can be of equal length or may have different lengths (e.g., as shown in FIG. 29Y) for example, the distal portion can be configured to split apart into two sub-portions comprising 60% and 40% respectively of their total combined length. Other implementations can include those with approximately 55%/45%, 65%/35%, 70%/30%, etc., ratios of lengths and the ratios can be determined in order to provide optimal anatomical coverage given the implantation location.

For example, the distal portion can be configured to split apart into two sub-portions having different lengths. In some embodiments, the electrodes can include a cathode located on a shorter sub-portion of the two different length sub-portions and an anode located on a longer sub-portion (as shown in FIG. 29Y), an anode located on a shorter sub-portion of the two different length sub-portions and a cathode located on a longer sub-portion, etc.

Similar to the embodiments described with reference to FIG. 24, the sub-portions can include parallel planar surfaces. Similar to other embodiments, these sub-portions can then form rectangular prisms including the two parallel planar surfaces. As shown in FIG. 32, the distal portion can be wider (W) than it is thick (T).

During deployment, the lead is advanced through the tip of the delivery system (described further below). After placement of the lead in the patient, the delivery system can then be withdrawn (e.g., as indicated by the direction of the arrow in FIG. 32). To facilitate withdrawal of the delivery system after the lead has been implanted, the proximal portion of the lead can be configured to be thinner than the distal portion of the lead (see, e.g., location 3200 in FIG. 32, identifying the location where the proximal portion of the lead thins compared to the distal portion of the lead). In this manner, the lead can proceed directly through the tip of the delivery system 3000.

It is contemplated that each of the split distal portions of the splitting lead designs disclosed herein may incorporate features described above in conjunction with non-splitting lead designs.

For example, the sub-portions can include distal ends 3050 having flexible portions so as to allow the distal ends to change course when encountering sufficient resistance traveling through the biological tissue of the patient. For example, if the distal ends encounter bone, muscle, etc., the flexible portions can allow the distal ends to still deploy within the patient without necessarily affecting or damaging the resisting biological tissue. Such flexible portions can include a material that flexes more easily relative to material of other areas of the sub-portions. The material can be rubber, soft plastic, etc., which may be more flexible than the materials used for the rest of the sub-portions (e.g., metal, hard plastic, etc.). The flexible portions can include one or more cutouts 3060, which can be one or more areas having a reduced cross section compared to other areas of the sub-portions. In other embodiments, the flexible portions can be configured to cause the distal ends to be biased to change course in a particular direction. For example, such biasing can include using flexible materials having different flexibility in different portions, reinforcements such as rods that prevent flexing in certain directions, etc.

The particular shape of the distal ends can vary but, as shown in FIG. 32, the distal ends can be at least partially paddle shaped. In other embodiments, they may be more pointed to have a triangular or wedge shape or may be more rectangular to form a rectangular prism similar to the majority of the distal portion as shown.

Some embodiments of splitting leads can implement the use of shape memory material to enable deployment in a particular manner or in particular directions. For example, the sub-portions can include a shape memory material configured to bend in a predetermined direction when the sub-portions exit the delivery system. In this way, the delivery system can contain the sub-portions until they clear the internal structure of the delivery system and they will then deploy in their respective predetermined directions. Examples of such predetermined directions can result in creating an acute angle shape between the sub-portions and the proximal portion. In some embodiments, the sub-portions can be further configured to move in a direction opposite the predetermined direction responsive to the shape memory material being heated to body temperature. For example, some implementations can benefit from having the lead held at a lower temperature for ease of loading into the delivery system and/or deployment. Once introduced into the body, after an appropriate length of time, the sub-portions would then heat to body temperature and as such would become deployed in a direction opposite the predetermined directions (e.g., toward the heart). In some implementations, movement in the direction opposite the predetermined direction can create a ninety degree shape, or an obtuse angle shape between the sub-portions and the proximal portion.

In some embodiments, for example, to assist in deployment through tissue that may provide resistance, the sub-portions of a splitting lead can include distal ends with distal tips 3070 that can be smaller than the distal ends (e.g., can be pointed or wedged-shaped, or have a ball shape, etc.). Some such implementations can also benefit by having distal tips configured to be more rigid compared to other portions of the distal end.

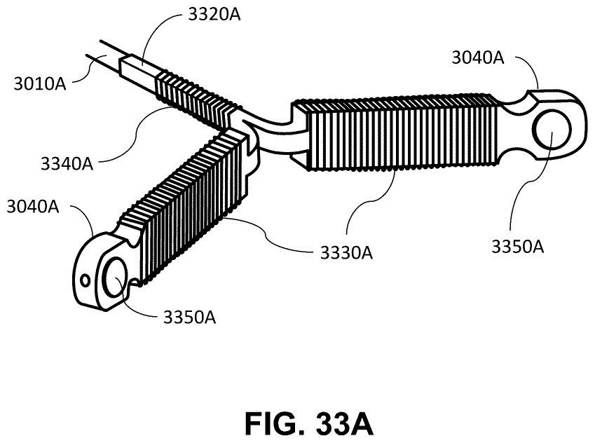

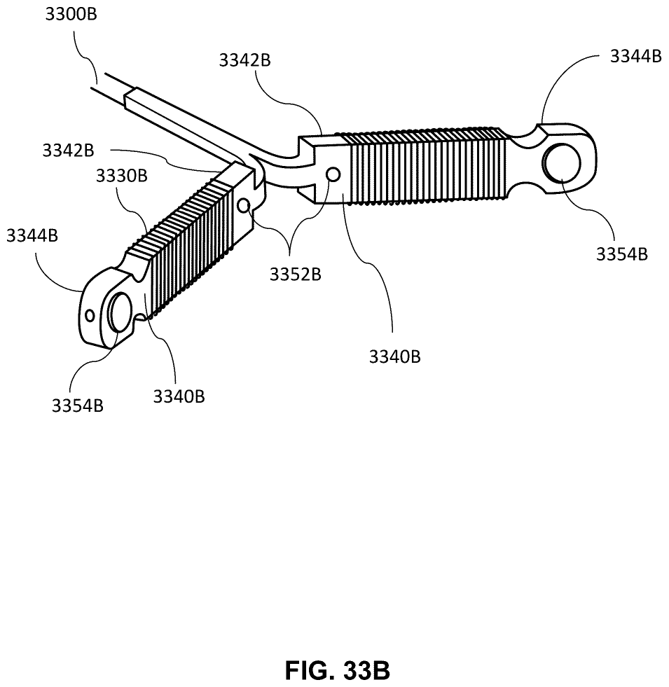

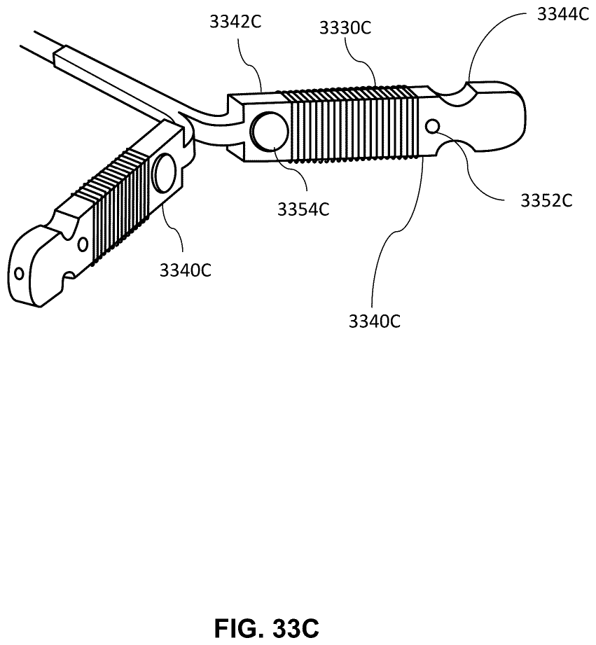

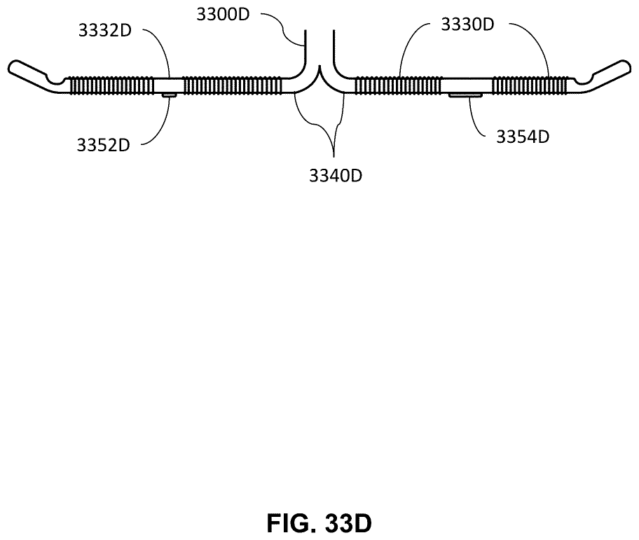

FIG. 33A illustrates an embodiment of a splitting lead that includes electrodes wrapped around the distal portion of the lead. A splitting lead 3010A may, for example, have electrodes 3330A wrapped around the sub-portions 3040A of the lead that travel in multiple directions during implantation (e.g., as a result of engaging with ramps, as described further below). In an embodiment where the sub-portions are rectangular prisms, the one or more electrodes wrapped around the sub-portions may be elliptical in shape. When an electrode is wrapped in such a way, the present disclosure refers to its shape as elliptical, even though the wrapped electrode may not be purely oval in shape—since such electrodes are still somewhat oval and are longer in one dimension (e.g., width dimension of the sub-portion) than in another dimension (e.g., thickness dimension of the sub-portion). See FIG. 32 for examples of the width W and thickness T of a sub-portion.

In addition to electrodes being wrapped around the sub-portions 3040A, electrode(s) may also be wrapped around a proximal part 3320A of the distal portion of the lead, specifically, the part of the distal portion that does not travel in different directions during implantation. Such wrapped electrodes 3340A can provide additional electrode surface area and may also be separately energized to deliver therapeutic energy along additional vectors. The present disclosure contemplates that such wrapped electrodes may be utilized for defibrillation and/or pacing.

The exemplary embodiment of FIG. 33A also depicts optional pacing electrodes 3350A located near the distal ends of sub-portions. In other implementations, the pacing electrodes 3350A may not be as close to the distal ends as they are in FIG. 33A (i.e., they may not be on the “flexible” portions previously-described but instead just proximal to those flexible portions). In still other implementations, the pacing electrodes may be located on only one of the sub-portions, for example, if that particular sub-portion will be located within the patient at a better location with respect to the heart for pacing. In some implementations, defibrillation electrodes can be placed proximal to the pacing electrodes.

FIG. 33B illustrates an embodiment of a splitting lead 3300B that includes proximally placed cathodes 3352B and a defibrillation electrode 3330B wrapped around the distal portion of the splitting lead. Exemplary lead 3300B can include a distal portion that is configured to split apart into sub-portions 3340B that travel in multiple directions during implantation into the patient. Defibrillation electrode 3330B can be located on one or both of sub-portions 3340B. When the present disclosure refers to a “defibrillation electrode,” such terminology may refer to the totality of a defibrillation electrode (i.e., all electrode material forming a single pole) or the term “defibrillation electrode” may refer to only a portion or segment of the overall defibrillation electrode. In the example of FIG. 33B, each sub-portion 3340B has a cathode 3352B at a proximal end 3342B of the sub-portion and an anode 3354B at a distal end 3344B of the sub-portion. In another example, the sub-portion could be configured to have a cathode at a distal end of the sub-portion and an anode at a proximal end of the sub-portion. FIG. 33C illustrates another such embodiment that includes distally placed cathodes 3352C and a defibrillation electrode 3330C wrapped around the splitting lead but in FIG. 33C, defibrillation electrode 3330C does not extend as far in the distal direction as in FIG. 33B. Such a configuration allows cathode 3352C to be placed on the distal end of the sub-portion, before the depicted cutouts.

FIG. 33D illustrates an embodiment of a splitting lead 3300D that includes electrodes between segments of a defibrillation electrode. Defibrillation electrode 3330D can be separated into two or more segments with electrodes placed in the gaps 3332D between defibrillation electrode segments. In the depicted embodiment, a sub-portion can have a cathode 3352D or an anode 3354D in a gap 3332D in the defibrillation electrode. For example, as shown, one sub-portion 3340D can have cathode 3352D and another sub-portion 3340D can have an anode 3354D. In various embodiments, gap 3332D can be sized to provide a separation of, e.g., at least 2 mm, at least 5 mm, at least 10 mm, etc., between the electrode (cathode or anode) and a defibrillation electrode segment, for example to prevent arcing or shorting.

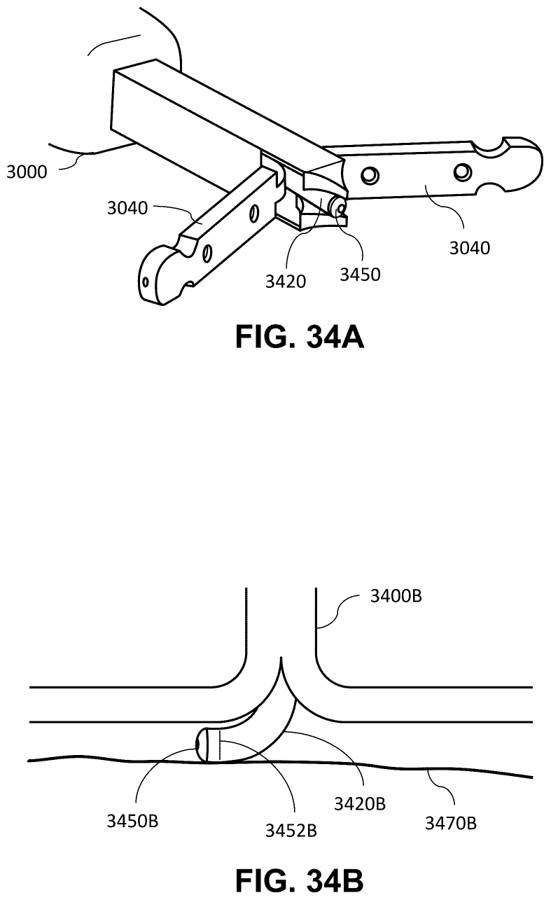

FIG. 34A illustrates an embodiment of a splitting lead further including an electrode extension. The exemplary lead can include a distal portion configured to split apart into sub-portions 3040 that travel in multiple directions during implantation into the patient. The lead can also include an electrode extension 3420 that increases a distance between an electrode 3450 and one or more other electrodes on the distal portion of the lead and/or facilitates contact of the electrode 3450 with patient tissue. This embodiment is similar to other splitting leads described herein and may also contain any of the features of such (e.g., wrapped electrodes, pacing electrodes on sub-portions, etc.). Electrode extension 3420 can be delivered via the delivery system 3000 as part of delivery of the splitting lead (which may include indentations in its sub-portions 3040 so that electrode extension 3420 better fits between the sub-portions 3040 when they fold together inside the delivery system). Electrode extension 3420 can extend and move along the main axis of the delivery system (e.g., straight down into the patient), and may be independently deployable and retractable/adjustable so the depth of the electrode tip can be independently set at the time of deployment. Consistent with discussions throughout the present disclosure, electrode extension 3420 may be used in conjunction with other electrodes and can provide additional vectors for the delivery of therapeutic energy. Electrode 3450 can be of any type, for example, a pacing electrode which may act as a cathode or an anode, in conjunction with another electrode elsewhere on the lead. In some embodiments, such an electrode 3450 and electrode extension 3420 form what is referred to herein as a central pacing electrode.

FIG. 34B illustrates an embodiment of a splitting lead 3400B with a flexible electrode extension. Flexible electrode extension 3420B is depicted in FIG. 34B as deflecting against patient tissue, e.g., pericardium 3470B. In some embodiments, electrode extension 3420B can include one or more cutouts that increase the flexibility of the electrode extension. In some embodiments, for example to facilitate maintaining contact with tissue when electrode extension is deflected into a bent position as shown, electrode 3450B can be a rounded electrode located at a distal tip of the electrode extension 3420B. In some embodiments, the rounded electrode 3450B can also have an electrically active segment 3452B that extends proximally from the tip, for example, along electrode extension 3420B. This is similar to the concept depicted in FIG. 29Y describing electrodes that extend along a side of a lead. It can be seen here that when electrode extension 3420B bends sufficiently, the rounded part of electrode 3450B may have reduced contact with patient tissue. However, by extending electrode 3450B proximally with electrically active segment 3452B (here meaning back along the body of electrode extension 3420B), the potential for greater electrode contact with patient tissue is increased.

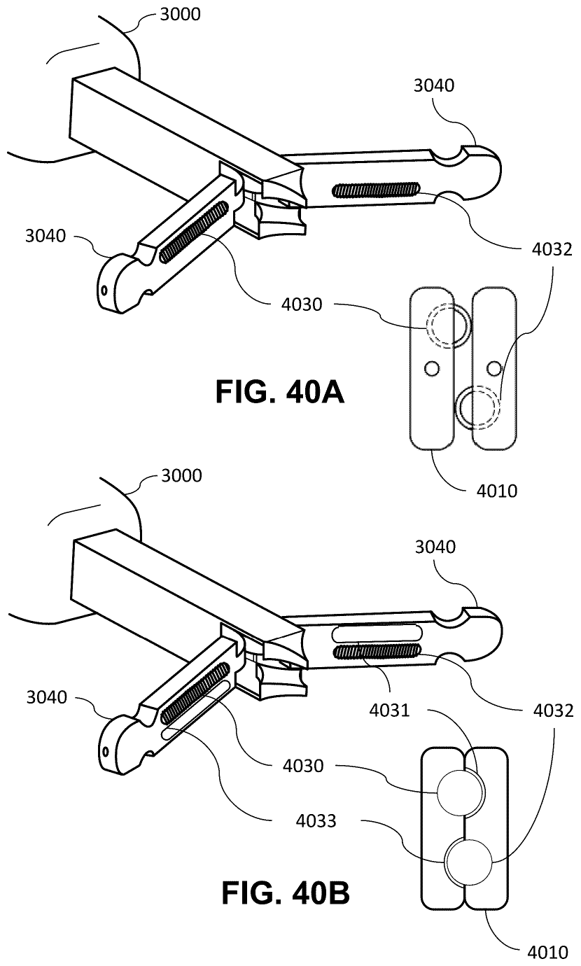

FIG. 35A illustrates an exemplary embodiment of a splitting lead that includes a protective collar for an electrode on an electrode extension (e.g., a pacing, sensing or defibrillation electrode). The embodiment also combines features of the splitting lead of FIG. 33A (having wrapped electrodes around the splitting lead's sub-portions 3040), the leads of FIGS. 34A and 54 (having a central electrode), and the splitting lead of FIG. 40B (having concavities 4031 and 4033 to allow the splitting lead to fully close and maintain a compact size). In this embodiment, the splitting lead can also include protective collar 3522A that can surround electrode extension 3520A. Such a protective collar can be configured to prevent the electrode from advancing too far into a patient or from perforating patient tissues due to the relatively sharp nature of the electrode by itself. The protective collar can also facilitate the application of contact pressure against patient tissues and can be made of an electrical insulator that can insulate patient tissues from the electrode. While one exemplary splitting lead/electrode configuration is shown in the embodiment of FIG. 35A, the protective collar and its related features may be utilized with any other embodiments disclosed herein that incorporate an electrode on an electrode extension.

With reference to the embodiment depicted in FIG. 35A, the protective collar 3522A can include a protective collar stopping foot 3524A having a laterally extending portion 3526A that can abut patient tissues at a desired location to prevent further inward deployment of the central pacing electrode. The distance between the distal face 3528A of the protective collar stopping foot and the tip of electrode 3450 can be selected to minimize the likelihood of tissue perforation and also to provide the desired contact pressure against patient tissues. Similar to the embodiment of FIG. 40B, the splitting lead embodiment in FIG. 35A can include various concavities 3529A in the lead body and/or sub-portions that are shaped to receive the protective collar, protective collar stopping foot, and/or the central pacing electrode such that the splitting lead can be fully closed.

FIG. 35B illustrates an exemplary embodiment with a tip electrode 3550B on a bridge 3522B between two sub-portions of a splitting lead 3500B. In some embodiments, an electrode extension can be a bridge 3522B connecting at least two of the sub-portions of lead 3500B. Bridge 3522B can be connected to proximal part(s) 3512B or (in the embodiment depicted) to distal part(s) 3514B. Similar to other electrode extension embodiments herein, a center portion 3524B of the bridge 3522B extends the tip electrode 3550B. In some embodiments, the center portion 3524B can have a surface area larger than that of tip electrode 3550B, similar to the wider electrode extension 2920S in FIG. 29S.

In some embodiments, lead 3500B can have proximal part 3512B that includes a gap 3526B, where bridge 3522B extends across the gap 3526B. As shown by the arrows in FIG. 35B, bridge 3522B can be configured to spread apart the sub-portions to distribute forces exerted on the center portion 3524B of the bridge 3522B (e.g., by the movement of a beating heart). In some embodiments, the distal portion of lead 3500B can include a cavity 3528B in a proximal part 3512B and/or a distal part 3514B (as shown) that is shaped to receive the bridge 3522B when the lead 3500B is in a loaded configuration.

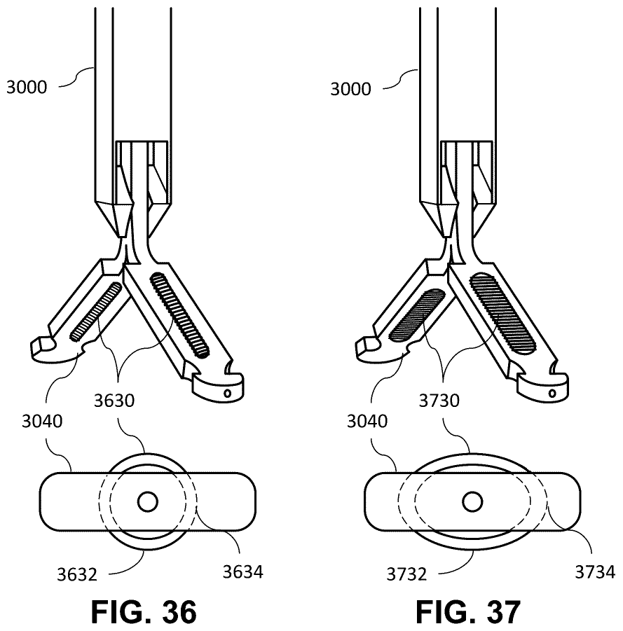

FIGS. 36 and 37 illustrate embodiments of splitting leads that have embedded electrodes (see 3630 and 3730 respectively). Such splitting-lead embedded electrodes may include the features of any of the embedded electrodes previously described with regard to FIG. 26.

The FIGS. 36 and 37 embodiments depict helical coils that are oriented in the longitudinal direction (i.e., along the lengthwise direction of the sub-portion). FIG. 36 depicts an embedded electrode 3630 with a circular shaped helical coil (i.e., as if wrapped around a cylindrical object) while FIG. 37 depicts an embedded electrode 3730 with an elliptical shaped helical coil (i.e., as if wrapped around an object with an elliptical cross-section). Other embodiments could have the embedded electrode be a solid electrode having a circular, elliptical (e.g., oval), or rectangular cross-section. Some elliptical or rectangular embodiments can beneficially provide greater surface area while keeping the thickness of the coil (e.g., in the semimajor direction or in a thinner direction) at a minimum to reduce the overall thickness of the directional lead.

As shown in the embodiments of FIGS. 36 and 37, the electrodes can be partially embedded in the sub-portions 3040 that travel in multiple directions during implantation. Similar to earlier embodiments, these partially embedded electrodes have an embedded portion 3634/3734 and an exposed portion 3632/3732. In the specific examples of FIGS. 36 and 37, the splitting leads have sub-portions that each comprise two parallel planar surfaces and the exposed portions of the embedded electrodes are on both of the planar parallel surfaces.

Simplified end views of the splitting lead sub-portions are shown in the insets of FIGS. 36 and 37, detailing parts of the embedded electrodes that are exposed, and parts that are embedded. As can be seen, the portion of the embedded electrodes that is exposed can have a similar surface area to the previously described electrodes. For example, the exposed portions can include at least 25%, 50%, 75%, etc., of the partially embedded electrode.

These embedded electrodes (also referred to herein equivalently as “partially embedded electrodes”) can include additional structural features for increasing surface area and/or current density as described above with reference to FIG. 26. Also, when referring herein to “embedded” electrodes, it is contemplated that some implementations may have a small amount of material between the conductive electrode and the patient that does not significantly reduce therapeutic energy and thus the “exposed” portion is still considered exposed. For example, there may be a thin layer of protective coating or the like between the electrode and the patient's tissue but this thin layer may cause no significant interference with the therapeutic energy provided via the embedded electrode.

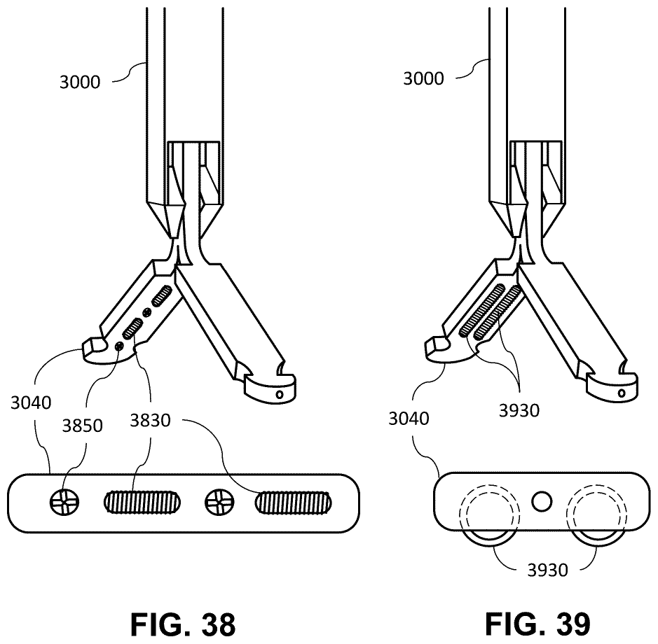

FIGS. 38 and 39 illustrate embodiments of embedded electrodes that are exposed only on only one side of the sub-portions. Such embedded electrodes will provide more directional stimulation, as discussed above. In the particular cross-sections of the depicted embodiments, the electrodes are helical coils and have an exposed portion on only one of two planar parallel surfaces.

FIG. 38 also illustrates that there may be multiple embedded electrodes 3830 on a single sub-portion 3040. FIG. 38 is similar to FIG. 36 but instead of one long embedded electrode, there are two shorter embedded electrodes that may be generally inline with each other (though some offset could be present in certain implementations). The embodiment of FIG. 39 provides an alternative design where two embedded electrodes 3930 are positioned side-by-side (e.g., parallel) on the same sub-portion 3040. Such designs can be beneficial in that the splitting of embedded electrodes into sections can provide for a greater number of vectors or can provide for alternative electrode surface areas and current densities. In other embodiments, there may be any number of electrodes besides two (e.g., three, four, five, etc.).

The particular embodiment depicted in FIG. 38 may employ electrodes 3830 for defibrillation and electrodes 3850 for pacing, although it is contemplated that each of the electrodes could be configured to be used for pacing and/or defibrillation. While not shown due to the perspective view, similar electrodes configurations can be utilized on both sub-portions. Moreover, other combinations of defibrillation and pacing electrodes, as discussed throughout this disclosure, may be chosen for the splitting leads.

FIG. 40A illustrates an embodiment of a lead having offset electrodes 4030 and 4032. This embodiment is similar to that shown in FIG. 39 but rather than having two embedded electrodes on each sub-portion 3040 there is one embedded electrode on each sub-portion and the exposed portions of the partially embedded electrodes are offset in order to avoid interference (e.g., contact) when the distal portion of the electrical lead is folded (i.e., before it splits apart into sub-portions that travel in multiple directions during implantation). A simplified view of a folded lead 4010 is depicted by the inset illustrating how such a lead has a smaller form factor than would be possible without such an offset. Additionally, as shown in FIG. 40B, sub-portions 3040 may include concavities 4031 and 4033 equally opposing the shapes of exposed electrodes 4030 and 4032. As shown by the inset section view, when the distal portion of the lead is folded, the exposed portions of the electrodes fit within the concavities of the opposing sub-portion, thereby creating an even smaller form factor when folded. As with other embodiments, the partially embedded electrodes can include pacing electrodes and/or defibrillation electrodes, as well as optionally having a pacing electrode extend between the sub-portions that travel in multiple directions during implantation.

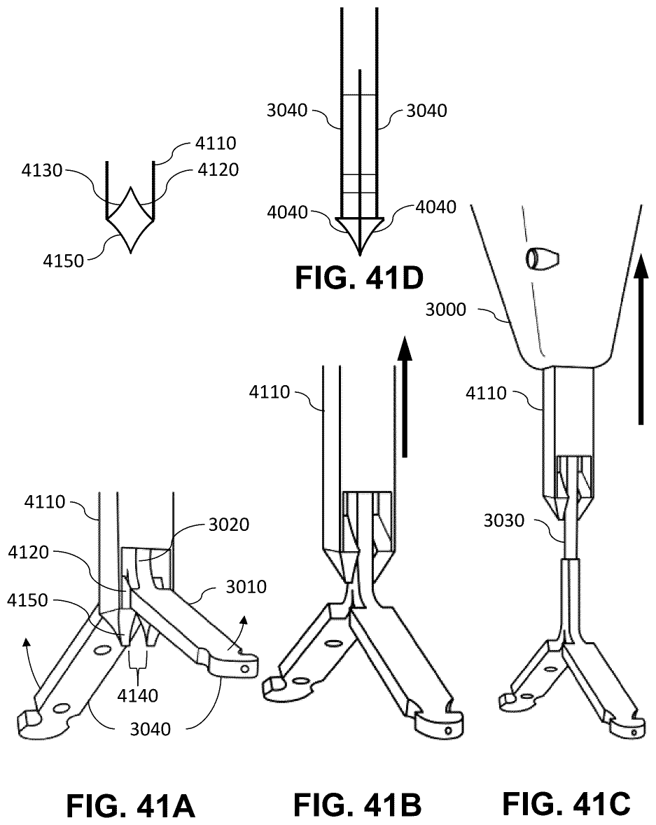

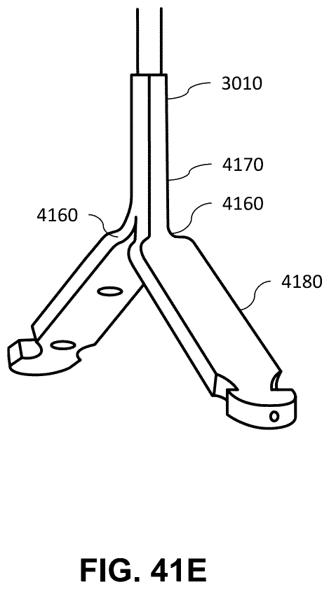

FIGS. 41A, 41B, and 41C illustrate portions of a delivery system deploying a component. The delivery system (for example, the delivery system 200 in FIGS. 9A-D or delivery system 3000 in FIG. 30A) can include a component advancer configured to advance the component into the patient. The delivery system can also include a handle configured to be actuated by an operator. The component advancer can be coupled to the handle and thereby configured to advance the component into the patient by applying a force to a portion of the component in response to actuation of the handle by the operator. Also, the component advancer can be configured to removably engage a portion of the component to deliver the component into the patient.

As depicted in the delivery system of FIG. 30A, the component can be a splitting lead 3010 having a proximal portion 3030 configured to engage a controller and a distal portion 3020 configured to split apart into sub-portions 3040 that travel in multiple directions during implantation into a patient. To facilitate the deployment of such a splitting lead, the delivery system can include, as shown in FIG. 41A, an insertion tip 4110 having a first ramp 4120 configured to facilitate advancement of a first sub-portion into the patient in a first direction. There can be a similar second ramp 4130 (shown in the cross-section view of the tip at the top of FIG. 41A) configured to facilitate advancement of a second sub-portion into the patient in a second direction.

As depicted in FIG. 41A, the first direction (i.e., the direction in which the first ramp advances the first sub-portion of the lead) can be opposite the second direction (i.e., the direction in which the second ramp advances the second sub-portion of the lead). In other words, the first direction can be 180° from the second direction. This directional split is also depicted in FIG. 31A.

In other embodiments, the angle between the first direction and second direction can be approximately 100°, allowing for placement of the sub-portions at least partially under the sternum. This directional split is also depicted in FIG. 31B. Other angles between the first direction and second direction (and their associated ramp configurations) are contemplated, for example, 90°, 110°, 120°, etc.

In some implementations, the delivery system can include a third ramp (e.g., in addition to the first and second ramps) configured to facilitate advancement of a third sub-portion into the patient in a third direction (e.g., 90° from the first and second directions). This can permit deployment of sub-portions approximately 90° apart and either parallel or perpendicular to the sternum of the patient.

In other implementations, at least the first ramp, and optionally the second ramp, may include a gap 4140 configured to facilitate removal of the delivery system after implantation of the splitting lead. An example of how gap 4140 can facilitate removal of the delivery system is depicted in the deployment sequence of FIGS. 41A, 41B, and 41C. The component (here a splitting lead) is shown in FIG. 41A having the sub-portions of the splitting lead engaging the first ramp and the second ramp to split apart in multiple directions. FIG. 41B then depicts a later stage in delivery showing the gap being wide enough to pass the proximal portion 3030 of the splitting lead, but still thinner than the width of the sub-portions of the splitting lead, which must engage the ramps in order to split off in different directions. Once the sub-portions have split apart such that they no longer engage the ramps, the delivery system can begin to withdraw over the proximal portion of the lead. FIG. 41C depicts the delivery system further withdrawn and the proximal portion 3030 of the lead being further exposed.

In another implementation, instead of the first and second ramps being at the same lengthwise position in the insertion tip (i.e., back, to back) the second ramp may be located at a more distal location than the first ramp so that advancement of the second sub-portion will be at a location deeper into the patient.

In some embodiments, the ramps may additionally include a taper at their proximal ends to widen the gap in that location. This widening can facilitate advancement of the component through the insertion tip by reducing the likelihood of the component getting stuck inside the gap.

To facilitate insertion of the delivery tool into patient tissue, the insertion tip may include a tissue-separating component 4150. As shown in FIGS. 41A, 41B, and 41C, the tissue-separating component can be wedge-shaped to separate and/or cut through tissue as needed for insertion. The tissue-separating component may also have a blunted distal end to reduce or avoid damage to tissue, blood vessels, etc. In the same manner as discussed above with regard to the ramps, the tissue-spreading component can include a gap configured to facilitate removal of the delivery system after implantation of the splitting lead.

Some embodiments of the insertion tip can include a movable cover configured to cover the gap during implantation. The movable cover can be configured to prevent tissue from accumulating in the gap when the insertion tip is pushed through patient tissue. Such movable covers can include, for example, a cover that can be pulled off when proper insertion depth is reached. In other examples, the cover can include a pivot, hinge, or flap to allow the movable cover to swivel out of the way of the component.

As depicted in FIG. 41D, other embodiments may incorporate a gap-filling component 4040 on the distal end of the splitting lead to fill the gap between the tissue-separating components. Gap-filling components 4040 may be incorporated on the distal ends of sub-portions 3040 such that, when the splitting lead is folded and loaded into the delivery system, the gap-filling components fit within and fill the gap of the tissue-separating component. Once inserted within the patient tissue, the gap-filling components are deployed with sub-sections 3040, as described previously with regard to FIG. 41A, thereby clearing the gap and allowing for proximal portion 3030 to travel through the gap, as shown in FIGS. 41B and 41C.

FIG. 41E illustrates a component (e.g., lead) having transition portions to aid in withdrawal of the component from a patient. Instead of having laterally extending surfaces that may contact patient tissue during removal, any of the components disclosed herein (e.g., leads such as splitting lead 3010 of FIG. 41C) can have transition portions 4160 that may push tissue aside during component removal. As shown in FIG. 41E, in some embodiments, the transition portions can be rounded or smoothly varying regions between a narrower part (e.g., proximal part 4170) and a wider part (e.g., distal part 4180). In other embodiments, the transition portions can have planar surfaces angled to push tissue aside during component removal.

As described above, in some implementations, system 200 (FIG. 3) includes the electrical lead 1600 (FIG. 16), handle 300 (FIG. 3), component advancer 302 (FIG. 3), first and second insertion tips 304, 306 (FIG. 3), and/or other components. First insertion tip 304 and second insertion tip 306 may be configured to close around a distal tip of the electrical lead when the electrical lead is placed within component advancer 302. First insertion tip 304 and second insertion tip 306 may be configured to push through biological tissue when in a closed position and to open to enable the electrical lead to exit from component advancer 302 into the patient. Component advancer 302, first insertion tip 304, and second insertion tip 306 may be configured to maintain the electrical lead in a particular orientation during the exit of the component from component advancer 302 into the patient. Also as described above, first insertion tip 304 may include a ramped portion configured to facilitate advancement of the component into the patient in a particular direction, and/or the electrical lead may be configured to bend in a predetermined direction after the exit of the component from the component advancer (e.g., because of its shape memory properties, etc.).

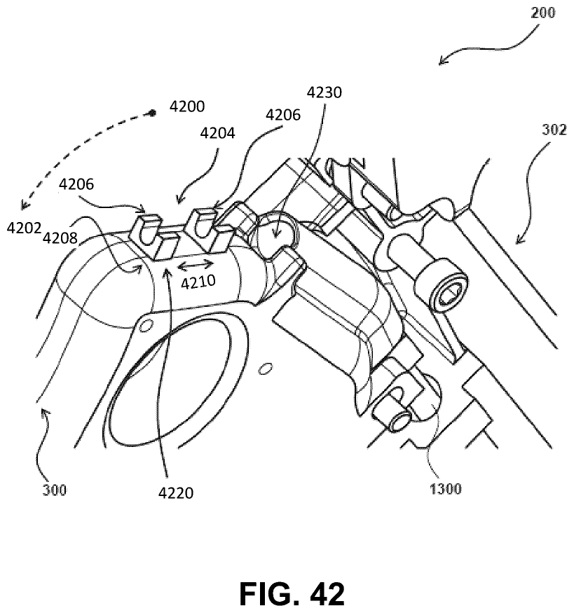

FIG. 42 illustrates components of delivery system 200 configured to load (or reload) a component (e.g., an electrical lead 1600 shown in FIG. 16) into delivery system 200. In some implementations, to facilitate reloading delivery system 200, an operator may thread proximal portion 1606 (FIG. 16) of lead 1600 backwards through insertion tips 304, 306 (FIG. 3), through pusher tube 1300 (in an implementation shown in FIG. 13) and out through an opening 4230 in handle 300. In some implementations, component advancer 302 may be configured to reload a component (e.g., an electrical lead) into delivery system 200. In such implementations, handle 300 may be configured to move from an advanced position 4200 to a retracted position 4202 to facilitate the reload of the component (e.g., the electrical lead).

In some implementations, handle 300 may include a dock 4204 configured to engage an alignment block coupled with the component (e.g., electrical lead) such that, responsive to handle 300 moving from advanced position 4200 to retracted position 4202, the engagement between dock 4204 and the alignment block draws the component into delivery system 200 to reload delivery system 200. As a non-limiting example using the implementation of component advancer 302 shown in FIG. 13-14, once the alignment block and electrical lead are properly seated within dock 4204, handle 300 may be re-cocked (e.g., moved from position 4200 to position 4202), which draws distal portion 1602 of electrical lead 1600 into delivery system 200 and closes insertion tips 304, 306 (FIG. 3).

In some implementations, dock 4204 may comprise one or more alignment and/or locking protrusions 4206 (the example in FIG. 42 illustrates two protrusions 4206) located on a portion 4208 of handle 300 toward component advancer 302. Locking protrusions 4206 may have a “U” shaped channel configured to receive a wire portion (e.g., part of proximal portion 1606) of an electrical lead 1600 (FIG. 16). Locking protrusions 4206 may have a spacing 4210 that corresponds to a size of an alignment block on the wire portion of electrical lead 1600 and allows the alignment block to fit between locking protrusions 4206 (with the wire portions resting in the “U” shaped channels of locking protrusions 4206).

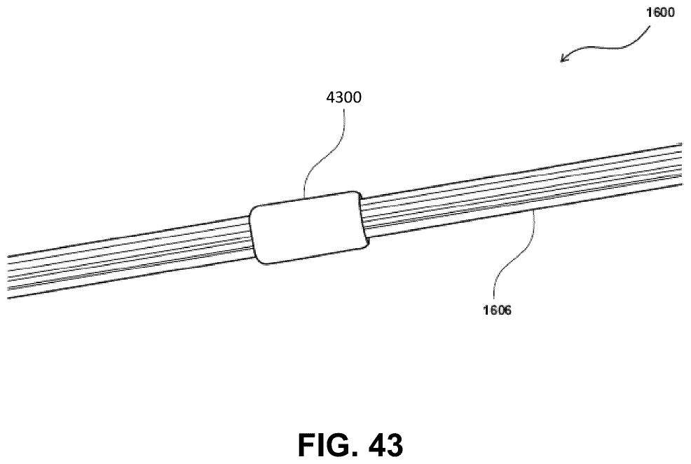

FIG. 43 illustrates an example of an alignment block 4300 coupled to proximal portion 1606 of an electrical lead 1600. Alignment block 4300 may have a cylindrical shape, for example, with a length matching spacing 4210 configured to fit between locking protrusions 4206 shown in FIG. 42.

Returning to FIG. 42, in some implementations, handle 300 may include an alignment surface 4220 configured to receive the proximal portion 1606 (FIG. 43) of electrical lead 1600 (FIG. 16) such that, responsive to handle 300 moving from the advanced position to the retracted position, the component is drawn into delivery system 200 to reload delivery system 200. In some implementations, alignment surface 4220 may be the same as surface 4208, but without locking protrusions 4206. In some implementations, an operator may hold proximal portion 1606 against alignment surface 4220, within a retention block 4206, with finger pressure while handle 300 moves from advanced position 4200 to retracted position 4202, for example. In some implementations, the alignment block 4300 may not be utilized.

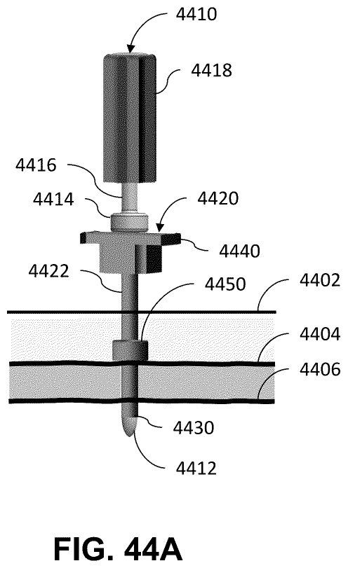

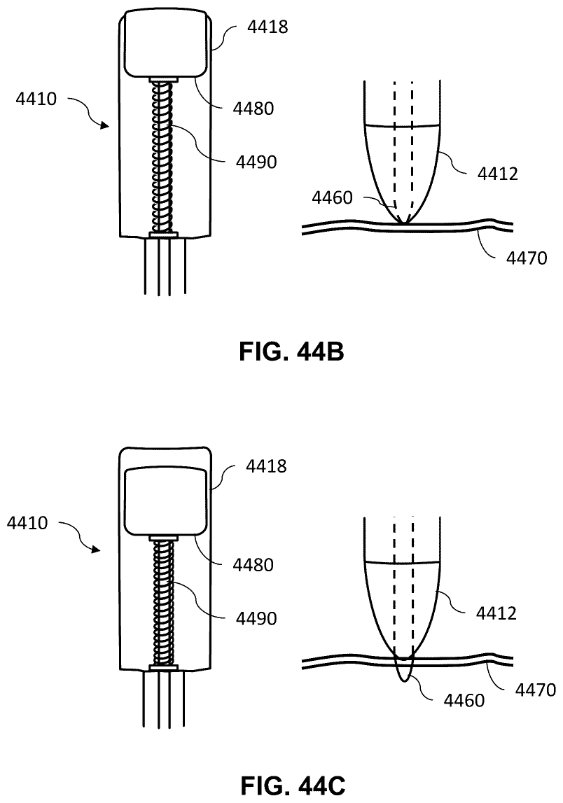

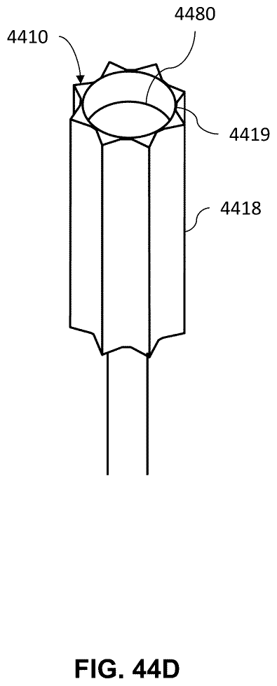

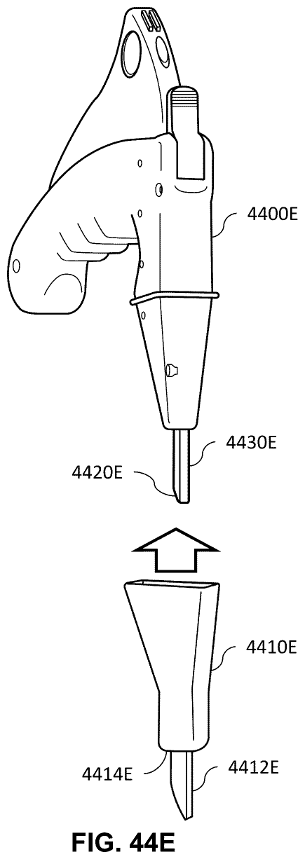

FIGS. 44A-52 illustrate an exemplary system and method for utilizing an insertion sheath while inserting an implantable lead into a patient. This method may be used with the splitting lead embodiments of the present disclosure and thus some element references will be made to the embodiments of a splitting lead depicted in FIGS. 41A-D and the associated delivery systems in FIGS. 30A/B.

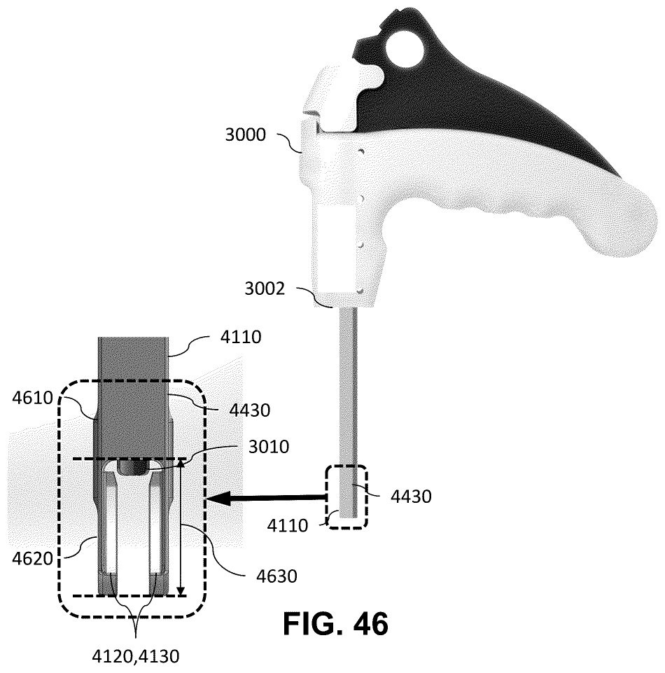

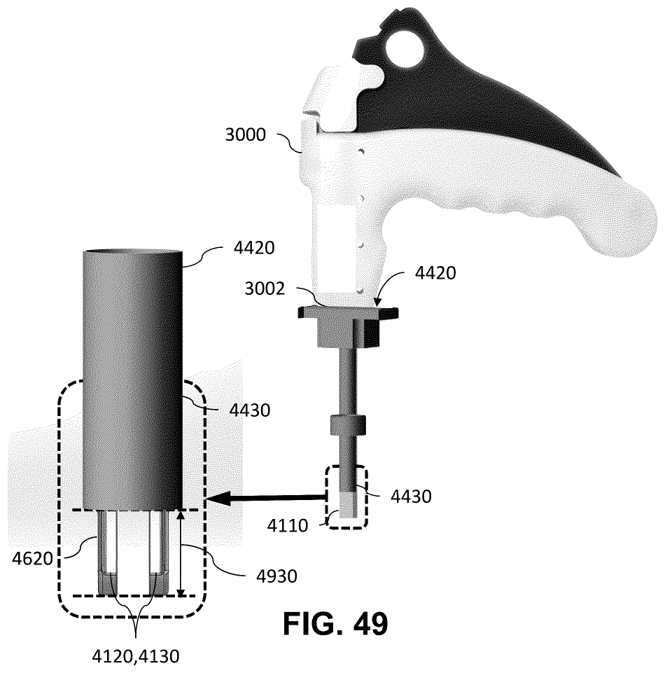

An advantage of the insertion sheath system includes having an “open” delivery tool insertion tip 4110, as shown in FIG. 46, to facilitate the loading of a lead into the delivery tool (see open area 4630) but then a more closed off insertion tip during lead delivery when the insertion tip is partially covered by an insertion sheath (see the more closed off area 4930 in FIG. 49). When the tip is more closed off during delivery, the deploying lead is better guided downward to the deployment ramps and any bulging of the lead above the ramps is constrained and avoided. The following descriptions for FIGS. 44A-52 disclose additional features of and methods of use for the insertion sheath.

FIG. 44A illustrates an exemplary insertion dilator 4410 for inserting an exemplary insertion sheath 4420 into a patient. The insertion sheath 4420 can be a sufficiently long tube to extend through the patient's skin 4402 and subsequent tissue layers (e.g., subcutaneous fascia 4404 and endothoracic fascia 4406) to reach a desired depth such as the anterior mediastinum. The insertion sheath body 4422 can have a hollow interior for receiving various components, such as an insertion dilator 4410 and also the insertion tip 4110 of a lead delivery tool.

The depicted insertion dilator 4410 can be utilized with insertion sheath 4420. When inserted into insertion sheath, the insertion dilator can extend out from the distal end 4430 of the insertion sheath such that a pointed end 4412 of the insertion dilator can act to separate patient tissue and penetrate the endothoracic fascia for the insertion sheath. Also, the insertion dilator can have an insertion dilator stopping foot 4414 that extends laterally from insertion dilator body 4416. The insertion dilator stopping foot can engage the insertion sheath (e.g., at insertion sheath hub 4440 extending laterally from insertion sheath body 4422 at a proximal end of the insertion sheath 4420) when a user pushes the insertion dilator and thereby pushes the insertion sheath into the patient. The insertion dilator body may also have a handle 4418 for gripping by the user. In other embodiments, rather than including a handle, the insertion dilator may be connected to another device (e.g., a robotic medical device) that would push the insertion dilator into the patient.

Features are depicted in FIG. 44A showing that the insertion dilator has penetrated the patient's skin and pushed the insertion sheath through the skin until it stopped at a particular depth where an insertion sheath stopping foot 4450 extending laterally from the insertion sheath body halts advancement at a particular location (e.g., abutting the subcutaneous fascia). The insertion sheath and its stopping foot are configured to result in the insertion tip of a delivery system inserted into the sheath being positioned at a particular depth within the patient (e.g., proximate the pericardium).

The present disclosure describes numerous devices that can be provided and/or used together to deliver and secure leads in a patient. In some embodiments, any combination of the disclosed devices can be provided in the form of a kit. For example, in some embodiments, a kit can contain a delivery system, insertion sheath, and an insertion dilator. In some embodiments, a kit can contain a delivery system and a dilator cap. In other embodiments, a kit can contain a delivery system, insertion sheath, an insertion dilator, a lead, and an anchor cap. It is contemplated that any of the particular delivery systems, insertion sheaths, insertion dilators, dilator caps, leads or anchor caps disclosed herein could be provided in the disclosed kits.

FIGS. 44B and 44C illustrate an exemplary use and structure of a puncture tip 4460 for an insertion dilator 4410. In some embodiments, the insertion dilator 4410 can include a puncture tip 4460 configured to extend distally from the pointed end 4412 of the insertion dilator 4410, which can be used to create an initial puncture in biological tissues 4470, through which the comparatively larger pointed end 4412 of the insertion dilator 4410 can follow. In various embodiments, the puncture tip 4460 can be fixed or can be retractable into the insertion dilator 4410 as shown in FIG. 44B.

In use, depressing an actuator can cause a retracted puncture tip 4460 to extend distally from the pointed end of the insertion dilator 4410 to create the initial puncture. Insertion dilator 4410 can include a button 4480 that causes advancement of the puncture tip 4460 from the pointed end 4412 of the insertion dilator 4410. The insertion dilator 4410 can penetrate until it abuts a particular tissue layer (e.g., the endothoracic fascia or any other tissue layer that may have increased resistance to the insertion dilator 4410). For example, a method of use can include puncturing the endothoracic fascia with the puncture tip 4460 extending distally from the pointed end of the insertion dilator 4410. The insertion dilator 4410 can then advance through the punctured endothoracic fascia.

In retractable embodiments, the insertion dilator 4410 can include a spring-actuated retraction mechanism having a spring 4490 operatively connected to the puncture tip 4460 and configured to retract the puncture tip 4460 into the insertion dilator. Button 4480 can be coupled to the spring-actuated retraction mechanism to cause the spring to compress and advance the puncture tip 4460 distally from the pointed end of the insertion dilator 4410. By including mechanical stops or selecting a particular spring, various embodiments of the insertion dilator can be configured to limit the extent of the puncture tip extension to a predefined amount. In some embodiments, the predefined amount can be, for example, 1, 2, 3, 5, 10 mm from the pointed end. The present disclosure also contemplates fixed puncture tip embodiments (i.e., not retractable) having the same predefined extension, due to the length of the puncture tip itself.

FIG. 44D illustrates an exemplary recessed button for the insertion dilator 4410. In some embodiments, button 4480 can be recessed into a handle 4418 of insertion dilator 4410. Such a button can be recessed, for example, 3, 5, 7, 9 mm, etc., from a top edge 4419 of handle 4418 so that a user must actively extend into the recess to actuate button 4480 to cause the puncturing with the puncture tip as described above.

In other embodiments, the insertion dilator 4410 can be configured to have exchangeable ends. For example, the pointed end can be removed and replaced with a different end having a puncture tip, or a blunt tip. The insertion dilator 4410 can be configured for exchangeable ends for example with screw threads, magnets, etc.

FIG. 44E illustrates a delivery system 4400E with an exemplary dilator cap 4410E. In an alternative embodiment, instead of using a dilator with an insertion sheath (as in FIG. 44A), a separate dilator cap can instead be used. The dilator cap can be placed over the distal end of a delivery system and can be pressed into a patient to separate tissue and create a hole for lead delivery. Once the hole in the tissue is created, the delivery system with cap can be withdrawn, the dilator cap can be removed, and the delivery system (loaded with a lead) can be inserted into the hole to deploy the lead.

The dilator cap 4410E can be utilized with any of the disclosed delivery systems. For example, in various embodiments, a delivery system can have a channel 500 between first and second insertion tips 304, 306 (as in delivery system 200 of FIG. 5), a unitary insertion tip 900 with a distal orifice 908 (as in delivery system 200 of FIG. 9B), an insertion tip 4110 with an opening for loading and deploying a splitting lead 3010 (as in delivery system 3000 in FIGS. 41A-C, etc.). Thus, in general (though the terminology may vary slightly with the embodiment), a delivery system 4400E can have an insertion tip 4430E configured to be loaded with a lead and configured to deploy the lead through a distal opening 4420E in the insertion tip.

Dilator cap 4410E can be configured to fit over the insertion tip 4430E and cover the distal opening 4420E in the insertion tip 4430E. In some embodiments, dilator cap 4410E can include a tissue-separating portion 4412E that is wedge-shaped. It can be seen from FIG. 44E that the dilator cap can be shaped to compliment a shape of the delivery system to engage the delivery system 4400E for advancing the dilator cap 4410E. The dilator cap can also include a shoulder 4414E configured to engage the delivery system for advancing the dilator cap. Thus, by pushing down on delivery system 4400E, it engages shoulder 4414E and causes advancement of dilator cap 4410E into the patient tissue while protecting the distal opening 4420E.

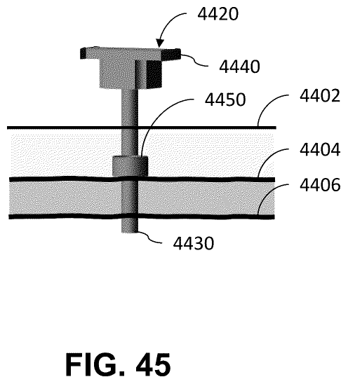

FIG. 45 illustrates the insertion sheath 4420 placed in the patient at the appropriate location and the insertion dilator removed. As shown, the insertion sheath stops where insertion sheath stopping foot 4450 meets subcutaneous fascia 4404. There can be a portion of the insertion sheath that extends into the patient from the subcutaneous fascia to slightly beyond the endothoracic fascia 4406 into the anterior mediastinum, which may be desired location of lead deployment. The insertion sheath can also include an insertion sheath hub 4440 that may house other features of the insertion sheath. For example, the insertion sheath hub can contain a protection valve configured to close around the delivery system to reduce or prevent air exchange through the hollow interior of the insertion sheath into/from the anterior mediastinum. The valve may be comprised of separating flaps or a membrane that can be penetrated by the delivery tool during insertion, but again come together when the delivery tool is removed to prevent air exchange through the hollow interior.

Once the insertion sheath 4420 is in place, lead delivery can commence. FIG. 46 illustrates exemplary features of a lead delivery system that facilitate loading a lead into the insertion tip. The distal end of the insertion tip 4110 can have two portions, an enclosed portion 4610 that constrains a lead and an open portion 4620 that includes ramp(s) (4120, 4130) that act to aid and deployment of the lead. The longitudinal distance where the lead is no longer constrained on all sides is referred to herein as the “window” 4630. As shown, with a larger window, it is easier to load the splitting lead 3010 (shown slightly protruding from within the insertion tip) because there is less friction between the splitting lead and the walls of the insertion tip. In this exemplary embodiment, the window 4630 can be larger than the longitudinal (vertical) extent of the ramp(s).

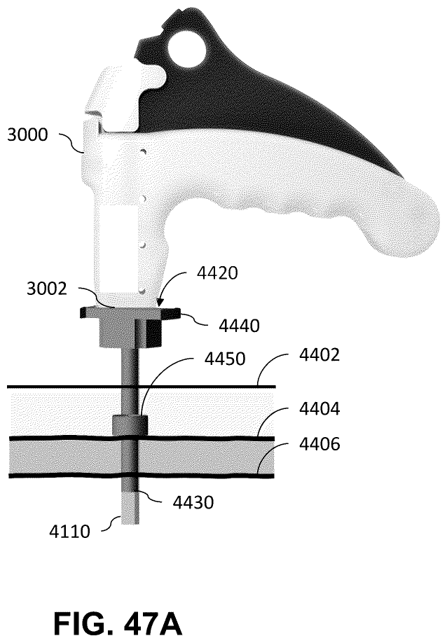

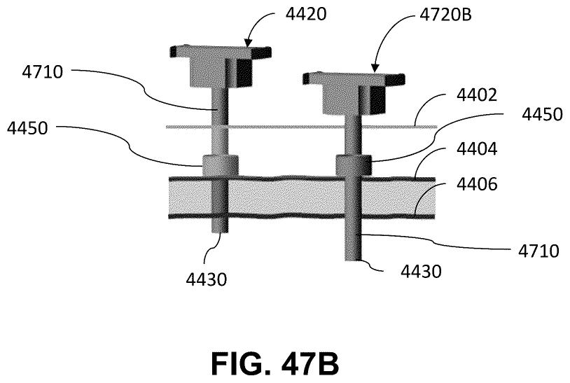

FIG. 47A illustrates a delivery system inserted into the insertion sheath. With the insertion sheath 4420 properly placed in the patient, delivery system 3000 can be inserted into the insertion sheath to reach the desired location in the patient. As shown, the delivery system 3000 can have a flat portion 3002 that abuts the proximal end of the insertion sheath causing a natural stop, with the insertion tip slightly extending beyond the distal end 4430 of the insertion sheath.

FIG. 47B illustrates exemplary embodiments of insertion sheaths that can act, given the selected dimensions of the sheath components, to place the delivery system insertion tip at the proper location for deployment of a lead. For example, the insertion sheath stopping foot 4450 can be located farther up insertion sheath shaft 4710 (as shown on sheath 4720B). This will cause the delivery system tip to exit the sheath at a deeper location into the patient, as shown. In another implementation, the length of insertion sheath shaft 4710 that is below the stopping foot 4450 is increased for greater insertion depth, but the length of the shaft above the stopping foot can remain the same. In this implementation, the insertion sheath used for deeper implantation will end up having a longer shaft than sheath 4420. The kits described herein as including a sheath could thus alternatively be provided with multiple sheaths configured to result in multiple implantation depths.

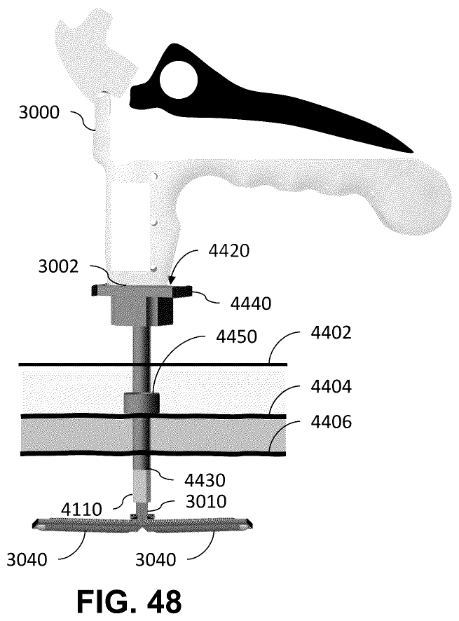

FIG. 48 illustrates an exemplary deployment of a splitting lead. While the present disclosure contemplates that the delivery systems herein can advance any of the disclosed leads through an insertion tip, FIG. 48 depicts one example of advancing a splitting lead. The delivery system 3000 can be activated by a user (e.g., by squeezing a handle, pointer, etc.) to advance the splitting lead 3010 through the insertion tip 4110. As shown in FIG. 48, and also described in previous embodiments, the splitting lead can then extend from the insertion tip with the sub-portions 3040 of the splitting lead separating in lateral directions into the patient.

FIG. 49 illustrates the insertion sheath creating a reduced window that improves deployment of the splitting lead. When advancing the splitting lead into tissues, the tissues may push back against the lead and cause unwanted or premature splitting of the lead and/or the splitting lead to “bulge” out prior to interaction with the ramps that are intended to guide proper lead deployment. In the expanded view of FIG. 49, the insertion sheath 4420 acts to reduce the window 4930, thereby forcing deployment of the lead to be at the proper location (e.g., starting the splitting at the start of the ramps rather than before them). It can be seen in the figure that the insertion sheath can be configured to decrease the size of the window because window 4930 now has a smaller distance between the distal end of insertion tip 4110 and the distal end of the insertion sheath 4420 (as contrasted with the larger window 4630 in FIG. 46).

In another embodiment, the enclosed portion of the insertion tip itself (see FIG. 46) can be configured to be extended distally, closer to the ramps, to make the window equivalent to that shown in FIG. 49. Such an embodiment may optionally be utilized without use of the insertion sheath.

In an alternative method for decreasing the size of a large lead loading window in order to prevent lead bulging during deployment, some embodiments can include (e.g., as part of a kit with a delivery system), a ring having a hollow interior shaped to receive the distal end of a delivery system insertion tip. The ring can be configured to generally constrain the lead in a manner similar to that of the insertion sheath, without the need for an insertion sheath. In particular, some such embodiments can have the ring being of a length that a distal end of the ring meets the beginning of ramps in an insertion tip. Examples of such lengths can include 15, 17, 19, 21, or 23 mm or as needed to abut the delivery system (e.g., at flat portion 3002) and have the distal end be at a given location relative to the ramps. In some embodiments, the ring can have a narrowing along an inner distal edge to provide a smoother transition for the lead as it exits the ring.

The sheaths and rings disclosed herein can be used both with delivery systems delivering splitting leads and other delivery systems (e.g., delivering non-splitting leads such as depicted in FIG. 28A).

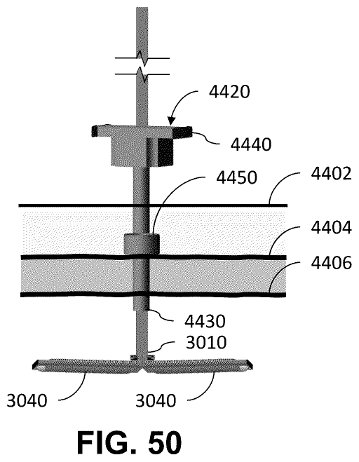

FIG. 50 illustrates removal of the delivery system and insertion tip. With splitting lead 3010 deployed, the delivery system 3000 and insertion tip 4110 can be withdrawn. With the insertion tip no longer between the splitting lead and the insertion sheath, the protection valve within the sheath can then seal against the lead body to continue to provide protection against air exchange.

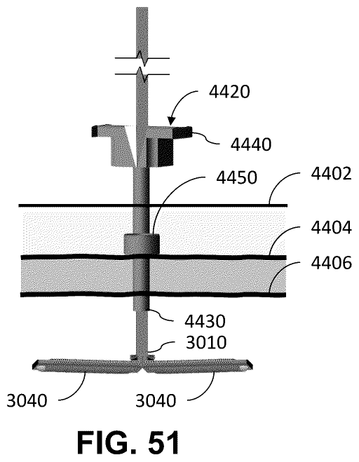

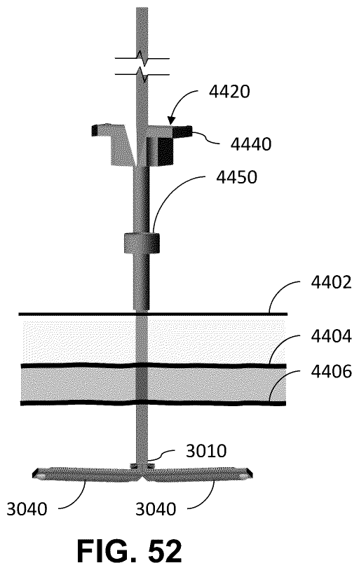

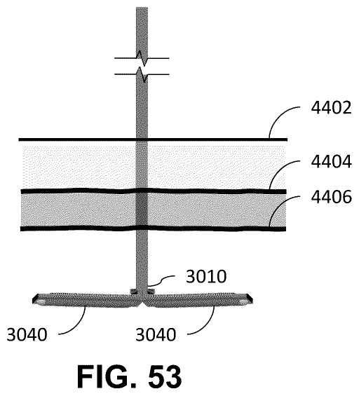

FIGS. 51-53 illustrate removal of an insertion sheath embodiment having separating portions. At this point in the delivery process, the insertion sheath 4420 can be removed from the patient. However, to reduce the contact between the insertion sheath and the lead 3010, some embodiments of the insertion sheath can be at least partially separable (FIG. 51) to effectively loosen the sheath contact around the splitting lead so as not to drag on or disturb the splitting lead when the insertion sheath is withdrawn (FIG. 52). In one embodiment, the insertion sheath can be configured to at least partially separate (e.g., crack open) along at least a portion of its length to facilitate removal over the lead. Examples of such separable portions can include at least the portion including insertion sheath hub 4440 containing the protective valve (to prevent the valve from grabbing onto the lead body during sheath removal). Other embodiments may also include portions of the lead body and/or the insertion sheath stopping foot being separable as well. In another embodiment, portions of the insertion sheath (as above) can be configured to fully separate into two or more pieces which can then be individually withdrawn around the lead body (e.g., by pulling the pieces somewhat to the sides and withdrawing). With the insertion sheath fully withdrawn (FIG. 53), the splitting lead 3010 can be prepared for use. In some embodiments, a lead anchor such as sutures or other methods of fixation can be utilized to fixate the splitting lead to the patient's tissues (e.g., to the subcutaneous fascia).

FIG. 54 illustrates a lead with suture holes 5460 for securing the lead to tissue. In some embodiments, suture holes 5460 may be located in a proximal part 5430 of the distal portion 5410 of lead (i.e., the portion that does not travel in a different direction during implantation). A physician may tie sutures through a patient's tissues and suture holes 5460 in order to better fix the orientation of the distal portion of the lead at implantation. The sutures may be tied to intercostal muscle, skin, or any other portion of the patient suitable for securing the lead. While one exemplary configuration is depicted in FIG. 54, any number and/or combination of suture holes and suture hole locations can be included in any of the lead embodiments detailed throughout the present disclosure. For example, such suture holes may be utilized with splitting leads as well. Furthermore, rather than complete suture holes, one or more grooves or notches may be located on the proximal part 5430 of the distal portion of the lead. Such grooves or notches provide indentations that may aid in securing of the lead to the patient's tissue.

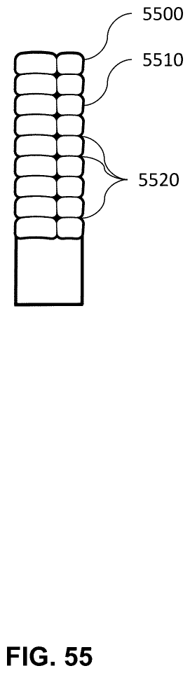

FIG. 55 illustrates a lead anchor 5500 for securing a lead. Lead anchor 5500 can be configured to slide over and securely fit on an elongated lead body (e.g., as shown in FIGS. 24, 29A, 35A, etc.). Lead anchor 5500 can be flexible/elastic plastic, rubber, or other deformable material that can stretch to cover a portion of the lead and remain secured when released. An exterior 5510 of lead anchor 5500 can have fixation features 5520 for facilitating fixation to patient tissue. In some embodiments, fixation features 5520 of lead anchor 5500 can include grooves, notches, or holes that facilitate suturing to biological tissue, with examples of grooves/notches depicted in FIG. 55.

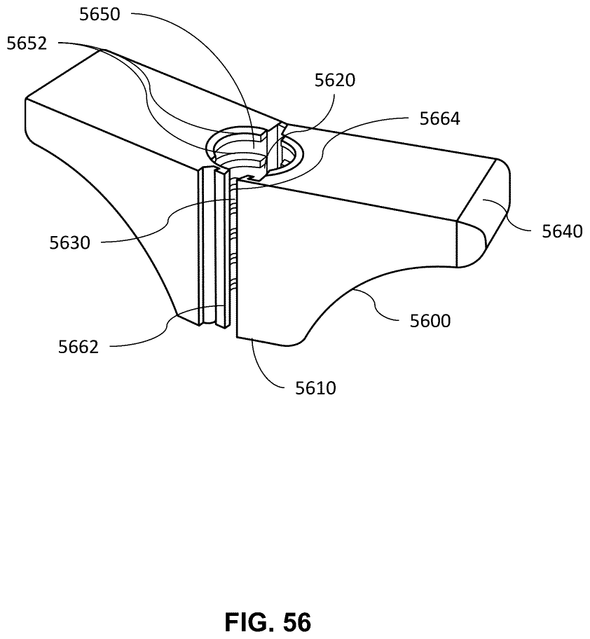

FIG. 56 illustrates a lead anchor insertion tool 5600 for pushing a lead anchor onto a lead. Lead anchor insertion tool 5600 can include a body 5610 having a bore 5620 extending longitudinally through body 5610 and shaped to accept a lead anchor (e.g., lead anchor 5500).

In some embodiments, the lead anchor insertion tool 5600 can have a textured surface 5652 on a surface 5650 of its bore. The textured surface can be complimentary to an exterior of the lead anchor (e.g., have a similar groove/notch pattern) or can have a different texture such as crossed scoring. Lead anchor insertion tool 5600 can include a handle 5640 extending in a lateral direction from the body to aid in pushing the lead anchor onto the lead.

Also, the lead anchor insertion tool's body 5610 can be configured to open along a longitudinal split 5630 and allow the lead anchor to be placed within the body. Such an opening can be configured by lead anchor insertion tool 5600 being thin and flexible in places or having an opening along a hinge, etc. In some embodiments, lead anchor insertion tool 5600 can have a locking clasp 5660 to hold the lead anchor insertion tool 5600 in a closed configuration. Locking clasp 5660 can include a male locking portion 5662 formed along a first half of longitudinal split 5630 and female locking portion 5664 formed along on a second half of the longitudinal split. Other locking mechanisms can include magnets, screws, etc.

In some embodiments, lead anchor insertion tool 5600 can alternatively be configured to grab onto a lead having an integrated lead anchor (described below) to further position the lead in the patient.

FIG. 57A illustrates a lead 5700A with indentations 5710A for securing the lead to tissue. In the depicted embodiment, the indentations 5710A can be formed in the lead itself in order to create an integrated lead anchor. In some embodiments, a method of securing such a lead can include inserting an electrical lead (e.g., having a distal portion with electrode(s) configured to generate therapeutic energy for biological tissue of the patient), where a proximal part 5712A of the distal portion can have grooves or notches (e.g., indentations 5710A). The method can also include securing the lead to patient tissue by suturing around the lead through the grooves or notches and into the biological tissue. As with other embodiments herein, the lead can also include a proximal portion coupled to the distal portion and configured to engage a controller configured to cause the electrodes to generate therapeutic energy.

FIG. 57B illustrates lead 5700A and an anchor cap 5720B. In some embodiments, to fill any empty space in the patient's tissue, an anchor cap 5720B can be utilized. A method of using such an anchor cap can include sliding an anchor cap 5720B over the distal portion of lead 5700A until a cap head 5722B of the anchor cap 5720B covers an opening in the patient tissue smaller than a width of the cap head 5722B. In some embodiments, the method can also include securing the cap head 5722B to the patient tissue utilizing one or more holes or notches 5724B in the cap head 5722B. In some embodiments, such an anchor cap can also be used with any of the leads disclosed herein, such as ones without indentations 5710A. Structurally, anchor cap can include an aperture 5726B having a shape corresponding to a cross-section of the proximal part of the lead over which the anchor cap is configured to be placed. The anchor cap can include a cap body 5723B and a cap head 5722B that extends laterally beyond the cap body. Anchor cap 5720A can also include one or more holes or notches 5724B on the cap head to facilitate suturing to patient tissue and/or to the lead.

In the following, further features, characteristics, and exemplary technical solutions of the present disclosure will be described in terms of items that may be optionally claimed in any combination:

Item 1: An electrical lead for implantation in a patient, the lead comprising: a distal portion comprising one or more electrodes that are configured to generate therapeutic energy for biological tissue of the patient; and a proximal portion coupled to the distal portion and configured to engage a controller, the controller configured to cause the one or more electrodes to generate the therapeutic energy.

Item 2: The electrical lead of Item 1, wherein the distal portion includes a balloon on an upper face of the distal portion, the balloon configured to cause a downward force against the distal portion when the lead is deployed.

Item 3: The electrical lead of any one of the preceding items, wherein the distal portion includes a spring on an upper face of the distal portion, the spring configured to cause a downward force against the distal portion when the lead is deployed.

Item 4: The electrical lead of any one of the preceding items, wherein the distal portion includes a wedge configured to extend from the distal portion and cause a downward force against the distal portion when the lead is deployed.

Item 5: The electrical lead of any one of the preceding items, the distal portion comprising a helical coil portion that is configured to be compressed prior to implantation and released when the lead is deployed.

Item 6: The electrical lead of any one of the preceding items, the distal portion comprising: a fixed portion configured to be affixed to a patient to retain the fixed portion in place; and an elastically deformable portion configured to maintain contact between an electrode and the biological tissue during heart movement when in a deployed configuration.

Item 7: The electrical lead of any one of the preceding items, wherein the fixed portion comprises one or more suture holes for suturing the fixed portion to the patient.

Item 8: The electrical lead of any one of the preceding items, wherein the fixed portion comprises one or more grooves for suturing the fixed portion to the patient.

Item 9: The electrical lead of any one of the preceding items, wherein the lead is shaped to have a first point of contact with the patient at a chest wall.

Item 10: The electrical lead of any one of the preceding items, wherein the lead is shaped to, when the lead is deployed, have a first point of contact with the patient at a chest wall and a second point of contact with the chest wall, the first point of contact being at a distal end of the distal portion and the second point of contact being at a proximal point on the distal portion, the electrode disposed between the proximal point and the distal end.

Item 11: The electrical lead of any one of the preceding items, wherein the distal portion is configured to include a connecting portion having a contacting edge extending from the distal portion towards the biological tissue when deployed and configured to, in operation, pull the distal portion towards the biological tissue by engagement of the contacting edge with the biological tissue.

Item 12: The electrical lead of any one of the preceding items, wherein the connecting portion is a suction cup.

Item 13: The electrical lead of any one of the preceding items, wherein the suction cup is configured to include, at deployment, an opening towards the biological tissue that, when a reduced pressure is formed in the suction cup, causes a downward pressure on the distal portion, toward the biological tissue.

Item 14: The electrical lead of any one of the preceding items, wherein connecting portion comprises one or more tines that are configured to engage the biological tissue and hold the distal portion and the one or more electrodes against the biological tissue.

Item 15: The electrical lead of any one of the preceding items, further comprising a stylet cavity formed within the distal portion and shaped to receive a stylet to facilitate delivery of the lead, wherein the connecting portion is configured to be held in an open configuration by the stylet and, when the stylet is removed, the tines extend through one or more apertures in the distal portion.

Item 16: The electrical lead of any one of the preceding items, wherein the one or more electrodes comprises multiple electrodes spaced along the distal portion.

Item 17: The electrical lead of any one of the preceding items, wherein the lead is configured to deliver the therapeutic energy with one or more sets of the multiple electrodes.

Item 18: The electrical lead of any one of the preceding items, further comprising a connector having multiple poles corresponding to the multiple electrodes, the connector configured to provide the therapeutic energy from a pulse generator to the one or more sets of the multiple electrodes.

Item 19: The electrical lead of any one of the preceding items, further comprising a manual switch that configures the connector to deliver the therapeutic energy through a selected set of the multiple electrodes.

Item 20: The electrical lead of any one of the preceding items, further comprising an electrically insulating portion around at least part of a circumference of the lead, the electrically insulating portion configured to insulate surrounding muscle and/or tissue from the therapeutic energy.

Item 21: The electrical lead of any one of the preceding items, the distal portion having a coil shape that spreads out the multiple electrodes when the lead is in a deployed configuration.

Item 22: The electrical lead of any one of the preceding items, the distal portion having a spiral shape that spreads out the multiple electrodes when the lead is in a deployed configuration.

Item 23: The electrical lead of any one of the preceding items, the lead comprising an electrode at the center of the spiral.

Item 24: The electrical lead of any one of the preceding items, the distal portion having a wavy shape that spreads out the multiple electrodes when the lead is in a deployed configuration.

Item 25: The electrical lead of any one of the preceding items, the distal portion being flexible and further comprising a stylet cavity shaped to receive a stylet to facilitate delivery of the lead.

Item 26: The electrical lead of any one of the preceding items, the distal portion comprising one or more barbs extending from the distal portion and shaped to engage the biological tissue.

Item 27: The electrical lead of any one of the preceding items, wherein the one or more electrodes comprises multiple electrodes; and wherein the distal portion of the lead includes an electrode extension having a tip electrode, the electrode extension configured to facilitate contact of the tip electrode with biological tissue of the patient when the lead is in a deployed configuration.

Item 28: The electrical lead of any one of the preceding items, wherein the distal portion of the lead includes a cavity in a proximal part and/or distal part of the distal portion that is shaped to receive the electrode extension when the lead is in a loaded configuration.

Item 29: The electrical lead of any one of the preceding items, the electrode extension coupled to a distal part of the distal portion and, in the deployed configuration, extending at an angle away from the distal part.

Item 30: The electrical lead of any one of the preceding items, the electrode extension coupled to a proximal part of the distal portion and, in the deployed configuration, extending at an angle away from the distal part.

Item 31: The electrical lead of any one of the preceding items, the electrode extension further including an elbow.

Item 32: The electrical lead of any one of the preceding items, the electrode extension coupled to a proximal part of the distal portion and, in the deployed configuration having a horizontal extension and a vertical extension.

Item 33: The electrical lead of any one of the preceding items, the electrode extension coupled to a proximal part of the distal portion and, in the deployed configuration, having a C-shape and comprising a vertical extension.

Item 34: The electrical lead of any one of the preceding items, the electrode extension coupled to a proximal part of the distal portion and, in the deployed configuration, the electrode extension ending flush with a distal part of the distal portion with only the tip electrode protruding beyond the distal part.

Item 35: The electrical lead of any one of the preceding items, the electrode extension coupled to a distal part of the distal portion and, in the deployed configuration, extending substantially coplanar to the distal part.

Item 36: The electrical lead of any one of the preceding items, the electrode extension coupled to and aligned with a distal part of the distal portion.

Item 37: The electrical lead of any one of the preceding items, wherein the electrode extension is wider than a width of the tip electrode.

Item 38: The electrical lead of any one of the preceding items, wherein a distal part of the lead is configured to include a heel portion to facilitate contact of an electrode located on the heel portion with biological tissue of the patient when the lead is in a deployed configuration.

Item 39: The electrical lead of any one of the preceding items, wherein the heel portion is formed by a bend in the distal part of the lead that facilitates contact of the electrode located on the heel portion with the biological tissue of the patient when the lead is in the deployed configuration.

Item 40: The electrical lead of any one of the preceding items, wherein a proximal part includes a bend to place a vertical portion of the proximal part closer to a distal tip of the lead when the lead is in a deployed configuration to facilitate contact of an electrode with biological tissue of the patient when the lead is in the deployed configuration.

Item 41: The electrical lead of any one of the preceding items, wherein the bend places the vertical portion approximately over an electrode on the distal part.

Item 42: The electrical lead of any one of the preceding items, wherein the bend places the vertical portion closer to the distal tip than an electrode on the distal part.

Item 43: The electrical lead of any one of the preceding items, wherein the proximal part includes an S-shape.

Item 44: The electrical lead of any one of the preceding items, the bend configured to increase the flexibility of the proximal part of the lead to facilitate maintaining contact with the biological tissue when the lead is in the deployed configuration.

Item 45: The electrical lead of any one of the preceding items, the proximal part including one or more grooves or holes for suturing the vertical portion to the patient.

Item 46: The electrical lead of any one of the preceding items, the distal portion comprising two sub-portions that extend in different directions when in a deployed configuration, the sub-portions being semi-rigid.

Item 47: The electrical lead of any one of the preceding items, wherein the two sub-portions have an angle of at most 60 degrees from an axis.

Item 48: The electrical lead of any one of the preceding items, wherein each of the two sub-portions include an anode and a cathode.

Item 49: The electrical lead of any one of the preceding items, the distal portion comprising three sub-portions that extend in different directions when in a deployed configuration, the sub-portions being semi-rigid.

Item 50: The electrical lead of any one of the preceding items, wherein the three sub-portions have an angle of at least 180 degrees between two sub-portions on either side of a third sub-portion.

Item 51: The electrical lead of any one of the preceding items, wherein a first sub-portion and a second sub-portion each include a cathode and a third sub-portion includes an anode.

Item 52: The electrical lead of any one of the preceding items, the distal portion configured to split apart into sub-portions that travel in multiple directions during implantation into the patient, wherein multiple sub-portions include a cathode and the lead including an anode proximate a central region of the lead where the sub-portions meet.

Item 53: The electrical lead of any one of the preceding items, the distal portion configured to split apart into sub-portions that travel in multiple directions during implantation into the patient, wherein multiple sub-portions include an anode and the lead including a cathode proximate a central region of the lead where the sub-portions meet.

Item 54: The electrical lead of any one of the preceding items, the distal portion configured to split apart into sub-portions that travel in multiple directions during implantation into the patient, wherein at least one of the sub-portions does not include any of the one or more electrodes.

Item 55: The electrical lead of any one of the preceding items, the distal portion configured to split apart into sub-portions that travel in multiple directions during implantation into the patient, wherein at least one of the sub-portions includes a laterally-extending portion.

Item 56: The electrical lead of any one of the preceding items, wherein the distal portion includes a laterally-extending portion.

Item 57: The electrical lead of any one of the preceding items, wherein the distal portion is configured to split apart into two sub-portions having different lengths.

Item 58: The electrical lead of any one of the preceding items, wherein the one or more electrodes includes a cathode located on a shorter sub-portion of the two different length sub-portions and an anode on a longer sub-portion.

Item 59: The electrical lead of any one of the preceding items, wherein the one or more electrodes includes an anode located on a shorter sub-portion of the two different length sub-portions and a cathode on a longer sub-portion.

Item 60: The electrical lead of any one of the preceding items, the distal portion configured to split apart into sub-portions that travel in multiple directions during implantation into the patient.

Item 61: The electrical lead of any one of the preceding items, further comprising a defibrillation electrode on a sub-portion.

Item 62: The electrical lead of any one of the preceding items, wherein a sub-portion has a cathode at a proximal end and an anode at a distal end.

Item 63: The electrical lead of any one of the preceding items, wherein a sub-portion has a cathode at a distal end and an anode at a proximal end.

Item 64: The electrical lead of any one of the preceding items, wherein a sub-portion has a cathode or an anode in a gap in the defibrillation electrode.

Item 65: The electrical lead of any one of the preceding items, the distal portion configured to split apart into sub-portions that travel in multiple directions during implantation into the patient, wherein the distal portion of the lead includes an electrode extension having a tip electrode, the electrode extension configured to increase a distance between the tip electrode and another electrode on the distal portion of the lead and/or facilitate contact of the tip electrode with biological tissue of the patient when the lead is in a deployed configuration.

Item 66: The electrical lead of any one of the preceding items, wherein the tip electrode is a central pacing electrode.

Item 67: The electrical lead of any one of the preceding items, wherein the electrode extension is flexible.

Item 68: The electrical lead of any one of the preceding items, the electrode extension comprising one or more cutouts that increase the flexibility of the electrode extension.

Item 69: The electrical lead of any one of the preceding items, wherein the electrode is a rounded electrode at a distal tip of the electrode extension.

Item 70: The electrical lead of any one of the preceding items, wherein the rounded electrode also extends proximally from the distal tip along the electrode extension.

Item 71: The electrical lead of any one of the preceding items, the electrode extension comprising a bridge connecting at least two of the sub-portions, wherein a center portion of the bridge extends the tip electrode.

Item 72: The electrical lead of any one of the preceding items, wherein the distal portion of the lead includes a cavity in a proximal part and/or distal part of the distal portion that is shaped to receive the bridge when the lead is in a loaded configuration.

Item 73: The electrical lead of any one of the preceding items, wherein the distal portion has a flat surface and the one or more electrodes are oriented at angle(s) to the flat surface.

Item 74: The electrical lead of any one of the preceding items, wherein the one or more electrodes include at least two electrodes at the angle(s) and offset along the length of the distal portion.

Item 75: The electrical lead of any one of the preceding items, wherein the distal portion has a flat surface and a side, and the one or more electrodes are at least partially on the flat surface and extend at least partially over the side.

Item 76: The electrical lead of any one of the preceding items, wherein the distal portion has a flat surface and a side, and the one or more electrodes are rounded to at least partially extend over the side.

Item 77: The electrical lead of any one of the preceding items, the distal portion comprising one or more radiopaque indicators that are distinctly visible to an imaging device.

Item 78: The electrical lead of any one of the preceding items, wherein the lead has at least two radiopaque indicators with one of the radiopaque indicators being at a distal end.

Item 79: The electrical lead of any one of the preceding items, wherein the radiopaque indicator at the distal end form an L-shape.

Item 80: The electrical lead of any one of the preceding items, wherein the distal portion includes channels for holding cables for the one or more electrodes and the channels are at different depths in the lead than the one or more radiopaque indicators such that the cables do not interfere with the one or more radiopaque indicators.

Item 81: A method comprising: inserting an insertion dilator into an insertion sheath such that the insertion dilator extends out from a distal end of an insertion sheath; penetrating patient skin with the insertion dilator to push the insertion sheath through the skin to reach a particular depth; removing the insertion dilator from the insertion sheath; inserting a delivery system into the insertion sheath; deploying a lead by advancing the lead through an insertion tip of the delivery system.

Item 82: The method of item 81, wherein the insertion dilator penetrates until the insertion dilator abuts the endothoracic fascia, the method further comprising: puncturing the endothoracic fascia with a puncture tip extending distally from a pointed end of the insertion dilator; and advancing the insertion dilator through the punctured endothoracic fascia.

Item 83: The method as in any one of the preceding items, further comprising depressing an actuator to cause a retracted puncture tip to extend distally from the pointed end of the insertion dilator.

Item 84: A method comprising: inserting an electrical lead comprising: a proximal portion configured to engage a controller, the controller configured to cause one or more electrodes to generate therapeutic energy; and a distal portion coupled to the proximal portion, the distal portion comprising: one or more electrodes that are configured to generate the therapeutic energy for biological tissue of a patient; and one or more grooves or notches in a proximal part of the distal portion; and securing the lead to patient tissue by suturing around the lead through the one or more grooves or notches and into the biological tissue.

Item 85: The method of item 84, further comprising sliding an anchor cap over the distal portion.

Item 86: The method as in any one of the preceding items, further comprising securing a cap head to the biological tissue utilizing one or more holes or notches in the cap head.

Item 87: An insertion sheath configured to receive a delivery system and facilitate positioning of an insertion tip of the delivery system within a patient, the insertion tip including a window through which a lead can be loaded, the insertion sheath comprising: an insertion sheath body having a hollow interior shaped to receive the delivery system; an insertion sheath hub extending laterally from the insertion sheath body at a proximal end of the insertion sheath; and an insertion sheath stopping foot extending laterally from the insertion sheath body.

Item 88: The insertion sheath of item 87, wherein the insertion sheath and the insertion sheath stopping foot are configured to result in the insertion tip being positioned at a particular depth within the patient.

Item 89: The insertion sheath as in any one of the preceding items, wherein the particular depth is proximate the pericardium.

Item 90: The insertion sheath as in any one of the preceding items, wherein the insertion sheath is further configured to decrease a size of the window.

Item 91: The insertion sheath as in any one of the preceding items, the insertion sheath hub comprising a valve configured to close around the delivery system to reduce air exchange through the hollow interior of the insertion sheath.

Item 92: The insertion sheath as in any one of the preceding items, further comprising a separable portion that is at least partially separable along at least a portion of a length of the insertion sheath.

Item 93: An insertion dilator configured to separate patient tissue and to be used with an insertion sheath, the insertion dilator comprising: an insertion dilator body having a handle, an insertion dilator stopping foot extending laterally and configured to engage the insertion sheath, and having a length such that a portion of the insertion dilator body extends beyond the insertion sheath; and a pointed end configured to separate the patient tissue.

Item 94: The insertion sheath of item 93, further comprising a puncture tip configured to extend distally from the pointed end of the insertion dilator.

Item 95: The insertion sheath as in any one of the preceding items, wherein the insertion dilator is configured to cause advancement of the puncture tip up to a predefined amount from the pointed end of the insertion dilator.

Item 96: The insertion sheath as in any one of the preceding items, wherein the predefined amount is 2 mm.

Item 97: The insertion sheath as in any one of the preceding items, further comprising a button that causes advancement of the puncture tip from the pointed end of the insertion dilator.

Item 98: The insertion sheath as in any one of the preceding items, wherein the button is recessed into the handle of the insertion dilator.

Item 99: The insertion sheath as in any one of the preceding items, wherein the puncture tip is retractable into the insertion dilator.

Item 100: The insertion sheath as in any one of the preceding items, further comprising a spring-actuated retraction mechanism having a spring operatively connected to the puncture tip and configured to retract the puncture tip into the insertion dilator.

Item 101: The insertion sheath as in any one of the preceding items, wherein the insertion dilator is configured for exchangeable ends.

Item 102: A kit comprising: a delivery system with an insertion tip configured to be loaded with a lead through a window, the delivery system further configured to deploy the lead through the insertion tip; an insertion sheath configured to receive the delivery system and facilitate positioning of the insertion tip of the delivery system within a patient, the insertion sheath comprising: an insertion sheath body having a hollow interior shaped to receive the delivery system; an insertion sheath hub extending laterally from the insertion sheath body at a proximal end of the insertion sheath; and an insertion sheath stopping foot extending laterally from the insertion sheath body; and an insertion dilator configured to separate patient tissue and to be used with the insertion sheath, the insertion dilator comprising: an insertion dilator body having a handle, an insertion dilator stopping foot extending laterally and configured to engage the insertion sheath, and having a length such that a portion of the insertion dilator body extends beyond the insertion sheath; and a pointed end configured to separate the patient tissue.

Item 103: The kit of item 102, wherein the insertion sheath and the insertion sheath stopping foot are configured to result in the insertion tip being positioned at a particular depth within the patient.

Item 104: The kit as in any one of the preceding items, wherein the particular depth is proximate the pericardium.

Item 105: The kit as in any one of the preceding items, wherein the insertion sheath is further configured to decrease a size of the window.

Item 106: The kit as in any one of the preceding items, the insertion sheath comprising a separable portion that is at least partially separable along at least a portion of a length of the insertion sheath.

Item 107: The kit as in any one of the preceding items, further comprising an anchor cap having an aperture with a shape corresponding to a cross-section of a proximal part of the lead over which the anchor cap is configured to be placed.

Item 108: The kit as in any one of the preceding items, the anchor cap comprising a cap body and a cap head that extends laterally beyond the cap body.

Item 109: The kit as in any one of the preceding items, the anchor cap comprising one or more holes or notches on the cap head to facilitate suturing to patient tissue and/or to the lead.

Item 110: A system comprising: a delivery system having an insertion tip configured to be loaded with a lead, the delivery system configured to deploy the lead through a distal opening in an insertion tip; and a dilator cap configured to fit over the insertion tip and cover the distal opening in the insertion tip.

Item 111: The system of item 110, the dilator cap comprising a tissue-separating portion that is wedge-shaped.

Item 112: The system as in any one of the preceding items, the dilator cap comprising a shoulder configured to engage the delivery system for advancing the dilator cap.

Item 113: The system as in any one of the preceding items, the dilator cap shaped to compliment a shape of the delivery system to engage the delivery system for advancing the dilator cap.

Item 114: A system comprising: a lead anchor configured to slide over and securely fit on an elongated lead body, an exterior of the lead anchor comprising fixation features for facilitating fixation to patient tissue.

Item 115: The system of item 114, wherein the fixation features of the lead anchor include one or more grooves, notches, or holes that facilitate suturing to patient tissue.

Item 116: A system comprising: a lead anchor insertion tool comprising a body having a bore extending longitudinally through the body and shaped to accept a lead anchor, wherein the body is configured to open along a longitudinal split and allow the lead anchor to be placed within the body.

Item 117: The system of item 116, the lead anchor insertion tool further comprising a handle extending in a lateral direction from the body.

Item 118: The system as in any one of the preceding items, the lead anchor insertion tool having a textured surface on a surface of the bore.

Item 119: The system as in any one of the preceding items, wherein the textured surface is complimentary to an exterior of the lead anchor.

Item 120: The system as in any one of the preceding items, the lead anchor insertion tool having a locking clasp comprising: a male locking portion formed along a first half of the longitudinal split; and a female locking portion formed along on a second half of the longitudinal split, the male locking portion and the female locking portion configured to hold the lead anchor insertion tool in a closed configuration.

Item 121: A method comprising utilization of any one of the preceding Items.

Item 122: A system comprising: an apparatus described in any one of the preceding Items.

Item 123: A computer program product comprising a non-transitory machine-readable medium storing instructions which, when executed by the at least one programmable processor, cause the at least one programmable processor to perform operations causing a method utilizing an apparatus as described in any one of the preceding items.

### Boilerplate

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” (or “computer readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” (or “computer readable signal”) refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, computer programs and/or articles depending on the desired configuration. Any methods or the logic flows depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

1. An electrical lead for implantation in a patient, the lead comprising:
a distal portion comprising one or more electrodes that are configured to generate therapeutic energy for biological tissue of the patient; and
a proximal portion coupled to the distal portion and configured to engage a controller, the controller configured to cause the one or more electrodes to generate the therapeutic energy.

2-26. (canceled)

27. The electrical lead of claim 1, wherein the one or more electrodes comprises multiple electrodes; and
wherein the distal portion of the lead includes an electrode extension having a tip electrode, the electrode extension configured to facilitate contact of the tip electrode with biological tissue of the patient when the lead is in a deployed configuration.

28. The electrical lead of claim 27, wherein the distal portion of the lead includes a cavity in a proximal part and/or distal part of the distal portion that is shaped to receive the electrode extension when the lead is in a loaded configuration.

29. The electrical lead of claim 27, the electrode extension coupled to a distal part of the distal portion and, in the deployed configuration, extending at an angle away from the distal part.

30. The electrical lead of claim 27, the electrode extension coupled to a proximal part of the distal portion and, in the deployed configuration, extending at an angle away from the distal part.

31. The electrical lead of claim 30, the electrode extension further including an elbow.

32. The electrical lead of claim 27, the electrode extension coupled to a proximal part of the distal portion and, in the deployed configuration having a horizontal extension and a vertical extension.

33. The electrical lead of claim 27, the electrode extension coupled to a proximal part of the distal portion and, in the deployed configuration, having a C-shape and comprising a vertical extension.

34. The electrical lead of claim 27, the electrode extension coupled to a proximal part of the distal portion and, in the deployed configuration, the electrode extension ending flush with a distal part of the distal portion with only the tip electrode protruding beyond the distal part.

35. The electrical lead of claim 27, the electrode extension coupled to a distal part of the distal portion and, in the deployed configuration, extending substantially coplanar to the distal part.

36. The electrical lead of claim 27, the electrode extension coupled to and aligned with a distal part of the distal portion.

37. The electrical lead of claim 27, wherein the electrode extension is wider than a width of the tip electrode.

38. The electrical lead of claim 1, wherein a distal part of the lead is configured to include a heel portion to facilitate contact of an electrode located on the heel portion with biological tissue of the patient when the lead is in a deployed configuration.

39. The electrical lead of claim 38, wherein the heel portion is formed by a bend in the distal part of the lead that facilitates contact of the electrode located on the heel portion with the biological tissue of the patient when the lead is in the deployed configuration.

40. The electrical lead of claim 1, wherein a proximal part includes a bend to place a vertical portion of the proximal part closer to a distal tip of the lead when the lead is in a deployed configuration to facilitate contact of an electrode with biological tissue of the patient when the lead is in the deployed configuration.

41. The electrical lead of claim 40, wherein the bend places the vertical portion approximately over the electrode on the distal part.

42. The electrical lead of claim 40, wherein the bend places the vertical portion closer to the distal tip than the electrode on the distal part.

43. The electrical lead of claim 40, wherein the proximal part includes an S-shape.

44. The electrical lead of claim 40, the bend configured to increase the flexibility of the proximal part of the lead to facilitate maintaining contact with the biological tissue when the lead is in the deployed configuration.

45. The electrical lead of claim 40, the proximal part including one or more grooves or holes for suturing the vertical portion to the patient.

46-80. (canceled)
